(19) United States
(12) Patent Application Publication
(10) Pub. No.: US 2022/0347146 A1
(43) Pub. Date: Nov. 3, 2022

(54) CANCER TREATMENT

(71) Applicant: AXELIA ONCOLOGY PTY LTD, Melbourne, Victoria (AU)

(72) Inventors: Christophe Demaison, Melbourne (AU); Christopher Smith, Melbourne (AU); Francesca Mercuri, Melbourne (AU); Tobias Bald, Melbourne (AU); Michael Bettess, Melbourne (AU)

(21) Appl. No.: 17/640,714

(22) PCT Filed: Sep. 4, 2020
(86) PCT No.: PCT/AU2020/050936

§ 371 Date: Mar. 4, 2022

(30) Foreign Application Priority Data
Sep. 4, 2019 (AU) 2019 903263
Dec. 20, 2019 (AU) 2019 904863

(51) Int. Cl. A61K 31/231; A61K 31/17; A61K 9/00; A61P 35/00

(52) U.S. Cl. CPC A61K 31/231; A61K 31/17; A61K 9/0024; A61K 9/0073; A61P 35/00

(57) ABSTRACT

Methods, compounds, compositions, and kits for the treatment of cancer, comprising TLR2 agonists, particularly di-palmitoyl-S-glyceryl-cysteine (Pam2Cys) derivatives and conjugates, are provided. In one aspect, methods of treating, preventing or minimizing the progression of cancer comprising the administration of a compound comprising a Pam2Cys moiety and a polyethylene glycol (PEG) are provided.

### CROSS REFERENCE TO RELATED APPLICATIONS

This application claims priority to Australian provisional patent application no. AU2019903263 (filed on 4 Sep. 2019) and Australian provisional patent application no. AU2019904863 (filed on 20 Dec. 2019). The entire contents of each of AU2019903263 and AU2019904863 is hereby incorporated by reference.

### FIELD OF THE INVENTION

The invention relates to methods, compounds, compositions and kits for the treatment and/or prevention of cancer.

### BACKGROUND OF THE INVENTION

Cancer is a group of diseases involving abnormal cell growth with the potential to invade or spread to other parts of the body. The typical hallmarks of cancer include abnormal cell growth and division, avoidance of programmed cell death, limitless number of cell divisions, increased blood vessel formation, and invasion of tissue and formation of metastases. Typical approaches to the treatment of cancer have historically included radiation therapy, chemotherapy, immunotherapy, and surgery.

Despite improvements in therapies for the treatment of cancer, the cancer mortality rate worldwide remains high and strategies to prevent cancer recurrence are still needed. In 2015, about 90.5 million people had cancer. About 14.1 million new cases occur a year (not including skin cancer other than melanoma). Cancer causes about 8.8 million deaths (15.7% of deaths) and the financial costs of cancer were estimated at $1.16 trillion USD per year as of 2010. Current therapeutic strategies against cancer frequently result in treatment failure, often due to the development of multiple malignancies and/or resistance to chemotherapy and radiotherapy.

There therefore remains a need for improved therapies for the treatment of cancer.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method of treating, preventing or minimising progression of cancer in a subject comprising administering a therapeutically effective amount of a compound disclosed herein to a subject, thereby treating, preventing or minimising progression of cancer in the subject.

In one aspect, the present invention provides a method of treating, preventing or minimising progression of cancer in a subject comprising administering a therapeutically effective amount of a compound described herein to a subject, or a pharmaceutically acceptable salt, solvate and/or prodrug thereof.

In an embodiment, there is provided a method of treating, preventing or minimising progression of cancer in a subject comprising the administration of a therapeutically effective amount of a compound described herein to the respiratory tract of a subject, thereby treating, preventing or minimising progression of cancer in the subject. In a preferred embodiment, the compound is administered by inhalation.

In some embodiments, the compound is a compound as defined by any one of formulas (I), (IA1), (IA2), (II), (III), (IV), (V), (VI), (VII), (VIII), (IX), (X), (XI), (XII), (XIII), (XIV), (XV), (XVI), (XVII), (XVIII) and (XIX) (collectively referred to herein as formulas (I)-(XIX)).

In some embodiments, the compound may comprise moiety A selected from A1′ and A2 as defined herein and PEG, wherein the moiety A and PEG are linked by a glycine, serine, homoserine, threonine, phosphoserine, asparagine or glutamine residue, or an ester of a glutamine residue.

In some embodiments, the compound may comprise or consist of partial structure A1Y′ or A2Y′:

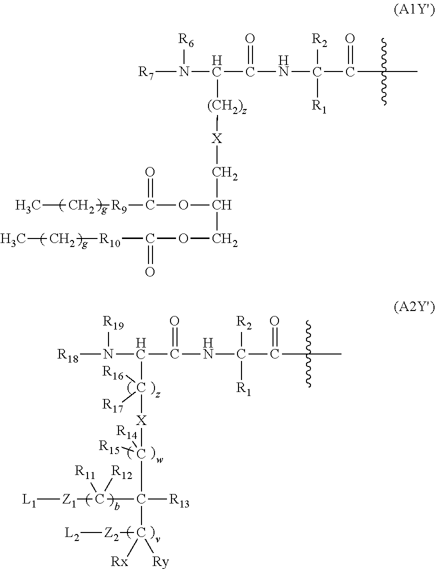

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
b and w are each independently an integer from 0 to 7 and v is an integer from 0 to 5, provided that:
the sum of b, v, and w is at least 3; and
the sum of b and w is from 0 to 7;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independentlyC₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and
A1Y′ or A2Y′ is covalently linked to polyethylene glycol (PEG),
or a pharmaceutically acceptable salt, solvate or prodrug thereof.

In some embodiments, the compound is selected from any of compounds A101-A114 and A201-A232.

Reference to a “compound of the invention” as used herein may refer to any of:
a compound of formulas (I)-(XIX);
a compound selected from any of compounds A101-A114 and A201-A232;
a compound comprising moiety A selected from A1′ and A2 as defined herein and a polyethylene glycol (PEG), wherein the moiety A and PEG are linked by a glycine, serine, homoserine, threonine, phosphoserine, asparagine or glutamine residue, or an ester of a glutamine residue; and/or
a compound comprising a partial structure of formula (A1Y′) or (A2Y′) covalently linked to a PEG.

In another aspect, the present invention provides a method of treating, preventing or minimising progression of cancer in a subject comprising the steps of:
identifying a subject having cancer; and
administering a therapeutically effective amount of a compound disclosed herein to a subject,

thereby treating, preventing or minimising progression of cancer in the subject.

In another aspect, the present invention further provides a method of increasing survival of a subject having cancer comprising administering a therapeutically effective amount of any compound described herein to a subject, thereby increasing survival of the subject having cancer.

In another aspect, the present invention further provides a method of minimising, reducing or preventing growth of a tumour in a subject having cancer comprising administering a therapeutically effective amount of any compound described herein to a subject, thereby minimising, reducing or preventing growth of a tumour in the subject having cancer.

In any aspect, the methods described herein further comprise identifying a subject having cancer. In an embodiment, the cancer may be pre-cancerous or non-metastatic. In another embodiment, the cancer may be malignant or metastatic.

In another aspect, the present invention further provides a method of minimising, reducing or preventing metastasis in a subject having cancer comprising administering a therapeutically effective amount of any compound described herein to a subject, thereby minimising, reducing or preventing metastasis in the subject having cancer. In a preferred embodiment, method minimises, reduces or prevents metastasis to the lung.

In any embodiment, the invention further provides a method of minimising, reducing or preventing metastasis in a subject having cancer comprising:
identifying a subject having a primary tumour capable of metastasising;
removing the primary tumour from the subject; and
administering a therapeutically effective amount of a compound disclosed herein to a subject,

thereby minimising, reducing or preventing metastasis in the subject having cancer.

In another aspect, the present invention further provides a method of minimising, reducing or preventing growth of a tumour in at least one site distant from the site of the primary tumour in a subject comprising administering a therapeutically effective amount of any compound described herein to a subject, thereby minimising, reducing or preventing minimising, reducing or preventing growth of a tumour in at least one site distant from the site of the primary tumour in the subject.

In another aspect, the present invention further provides use of any compound disclosed herein in the preparation of a medicament for treating, preventing or minimising progression of cancer in a subject.

In another aspect, the medicament is for any of the methods described herein including the following:
increasing survival of a subject having cancer;
minimising, reducing or preventing growth of a tumour in a subject;
minimising, reducing or preventing metastasis in a subject having cancer; or
minimising, reducing or preventing growth of a tumour in at least one site distant from the site of the primary tumour.

In any aspect of the invention, any medicament described herein is suitable for administration intraperitoneally, intratumorally, topically, orally, intravenously, subcutaneously or intramuscularly. Preferably, any medicament described herein is suitable for administration intravenously, or to the respiratory tract, preferably by inhalation. In one embodiment, the medicament may be suitable for formulation as a nasal spray or as nasal drops.

In another aspect of, the present invention further provides any compound disclosed herein for use in treating, preventing or minimising progression of cancer in a subject. In a further embodiment, any compound disclosed herein is for use in any of the methods described herein including the following:
increasing survival of a subject having cancer;
minimising, reducing or preventing growth of a tumour in a subject;
minimising, reducing or preventing metastasis in a subject having cancer; or
minimising, reducing or preventing growth of a tumour in at least one site distant from the site of the primary tumour.

In an aspect of the invention, the compound for use is suitable for administration intraperitoneally, intratumorally, topically, orally, intravenously, subcutaneously or intramuscularly. Preferably, the compound for use is suitable for administration intravenously, or to the respiratory tract, preferably by inhalation. In one embodiment, the compound may be suitable for formulation as a nasal spray or as nasal drops.

In another aspect, the present invention further provides use of any compound disclosed herein for use in treating, preventing, or minimising progression of cancer in a subject. In a further embodiment, use of any compound disclosed herein is for:
increasing survival of a subject having cancer;
minimising, reducing or preventing growth of a tumour in a subject;
minimising, reducing or preventing metastasis in a subject having cancer; or
minimising, reducing or preventing growth of a tumour in at least one site distant from the site of the primary tumour.

In any aspect of the invention, any compound disclosed herein is administered once. In another embodiment, any compound disclosed herein is administered two, three, four or more times to the subject.

In any aspect of the invention, any compound disclosed herein may be administered by any known administration routes in the art including locally or systemically. For example, topically, orally, intranasally, inhalation, intravenously, subcutaneously, intratumorally or intramuscularly. Preferably, any compound disclosed herein is administered intravenously, intratumorally or by inhalation.

In any aspect of the invention, the amount of any compound disclosed herein may be administered may be in the range of about 250 nmoles/kg body weight/dose to 0.005 nmoles/kg body weight/dose. Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, 5 nmoles, 2.5 nmoles, 0.5 nmoles, 0.25 nmoles, 0.1 nmoles or 0.05 nmoles/kg body weight/dose of the compound.

In any aspect of the invention, the amount of any compound disclosed herein may be administered may be in the range of about 0.1, 0.2, 0.3, 0.4, 0.5, 0.6, 0.7, 0.8, 0.9, 1.0, 5, 10, 15, 20, 25, 30, 35, 40, 45, 50, 55, 60, 65, 70, 75, 80, 85, 90, 95 or 100 μg/kg or more.

In any aspect of the invention, the cancer is selected from the group consisting of breast cancer, colorectal cancer, adenocarcinomas, mesothelioma, bladder cancer, prostate cancer, germ cell cancer, hepatoma/cholongio carcinoma, neuroendocrine cancer, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours or Wilm's tumour. In a preferred embodiment, the cancer is melanoma, breast, fibrosarcoma or colon cancer.

In any aspect, the cancer may be immunogenic (immune cell rich) or poorly immunogenic (immune cell poor).

In any embodiment, the compound to be administered may be any one of compound 1, compound 2, compound 3, compound 4, compound 5, compound 6, compound 9, compound 10, compound 11, compound 12, compound 13, compound 14, compound 15, compound 16, compound 17 as described herein. In another embodiment, the compound to be administered may be compound A108, A102, or A103.

In any aspect, any compound described herein may be administered in a composition. Typically, the composition further comprises a pharmaceutically acceptable carrier, diluent or excipient. The composition may be formulated for respiratory, intraperitoneal, intratumoural or intravenous administration to the subject. In other words, the composition is suitable for administration intratumourally, intravenously or to the respiratory tract. In any embodiment, the composition comprises, consists essentially, or consists of any compound described herein, and a pharmaceutically acceptable carrier, diluent or excipient.

In another embodiment of the invention, the composition is formulated for administration to the respiratory tract, for example by inhalation or intranasally. In one embodiment, the composition is formulated for administration as a nasal spray or as nasal drops.

In any aspect of the invention, any compound described herein may be administered as the only active agent, for example only pharmaceutically active agent. In other words, the compound may be administered free of any other compounds for treating, preventing or minimising the progression of cancer. In this embodiment, the present invention provides a method of treating, preventing or minimising progression of cancer in a subject consisting, or consisting essentially of administering a therapeutically effective amount of any compound disclosed herein to a subject, thereby treating, preventing or minimising progression of cancer in the subject.

In any aspect of the invention, any compound described herein is not administered with, or a composition comprising a compound described herein does not contain:
an antigen;
a peptide antigen; or
a T-helper antigen.

In one aspect, the composition may not activate a cell mediated immune response. In another aspect, any compound described herein is not administered with a cell penetrating peptide. In another aspect, the compound does not comprise an electrostatically associated charged antigen.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Compound 1 has anti-tumour activity in the MC38 model. A. Experimental setting: MC38-bearing WT mice were treated intratumorally with indicated doses of compound 1 or control. Tumour growth was measured and monitored over time. B. Corresponding mean tumour growth curves (mean±SEM.; n=10 for all groups). C. Kaplan-Meier survival curves (n=10 for all groups; Log rank test).

FIG. 2. Compound 1 has anti-tumour activity in the B16F10 model. A. Experimental setting: B16F10 melanoma bearing WT mice were treated intratumorally with indicated doses of compound 1. Tumour growth was measured and monitored over time. B. Corresponding mean tumour growth curves (mean±SEM.; n=10 control, n=10 for 25 μg compound 1 and n=9 for 50 μg compound 1).

FIG. 3. Compound 1 increases survival in the B16F10 model. Kaplan-Meier survival curves (n=10 control, n=10 for 25 μg compound 1 and n=9 for 50 μg compound 1; statistics; Log rank test).

FIG. 4. Systemic delivery of compound 1 inhibits spontaneous 4T1.2 breast cancer metastasis. A. Experimental setting: Balb/c mice were injected with 4T1.2 breast cancer cells. Primary tumours were surgically removed and mice treated as indicated. The number of metastases were counted on lung surfaces. B. Quantification of the number of 4T1.2 lung metastasis in indicated cohorts C. Combined analysis of experiment 1 and 2 (mean±SEM.; n=16 in each group; student's t-test).

FIG. 5. Compound A108 has anti-tumour activity in the immune cell-rich MC38 model. A. MC38 melanoma bearing WT mice were treated intratumorally (i.t.) with 25 ug of compound A108. Tumour growth was measured and monitored over time. The dashed lines indicate dosing regimen (every 2-days, three doses). B. Kaplan-Meier survival curves of the effect of compound A108 on survival.

FIG. 6. Compound A108 administered via the i.t or i.p route has anti-tumour activity in the immune cell-rich MC38 model in large tumours. Intra-tumoral administration with compound A108 every two days (days 1, 3, 5, 7, 9, 11 and 13). Tumour growth was measured and monitored over time. Body weights and tumour volumes were measured twice weekly. A) Demonstrates the anti-tumour effect of compound A108, over the treatment period when the compound is administered i.t. B Demonstrates the anti-tumour effect of compound A108, over the treatment period when the compound is administered i.p. 2-way ANOVA with Tukey's multiple comparison test. Treatment groups compared to each other within a specific time-point. C. Compound A108 yields a better effect on tumour growth than Pam3CysSK4 over 12 days in the MC38 large tumour model. Day 9 significant differences: Vehicle vs compound A108, **p=0.0040; compound A108 vs Pam3CysSK4, *p=0.0112; Day 12 significant differences: Vehicle vs compound A108, **p=0.0012; compound A108 vs Pam3CysSK4, *p=0.0080. D. Compound A108 yields an effect on tumour growth in the MC38 large tumour model that is at least as comparable as the effect of commercially available anti-PD1 antibodies.

FIG. 7. Compound A108 is effective in the WEHI-164 cancer model when administered intraperitoneally or intravenously. A) Experimental timeline indicating the number of days post WEHI-164 inoculation each treatment commenced. B) Mean tumour growth for each treatment group when administered intravenously, with standard error of the mean displayed (Welch's t test compared to control group). C) Corresponding Kaplan-Meier survival curves. D) Mean tumour growth for each treatment group when administered intraperitoneally, with standard error of the mean displayed (Welch's t test compared to control group). E) Corresponding Kaplan-Meier survival curves.

FIG. 8. Systemic delivery of compound A108 inhibits growth rate of EMT6.5 tumours. A) EMT6.5 tumour growth rates in mice treated with compound A108 or vehicle control. n=6 mice per therapeutic arm. Differences between tumour growth rate was evaluated by linear regression; B) Primary tumour weight at experimental end point. C) Evaluation of lung metastatic burden by RTQ-PCR assay. N=6 mice per therapeutic arm. Unpaired, two-sided t-tests were performed to determine significance; P values as indicated. D) Compound A108 leads to an extension in survival. Kaplan-Meier survival plot indicates better prognosis for animals treated with compound A108. Log-rank test. E) Effect of compound A108 compared to the effect of PD-1 on tumour volume.

FIG. 9. Repetitive intranasal administration of compound A108 reduces lung metastasis in a dose dependent manner in the 4T1.2 model. A,B) Experimental setting: 4T1.2 breast cancer cells were injected into a mammary fat pad. After resection of the tumour mice were treated as indicated. Finally, the number of lung metastasis was assessed in each mouse (A 2.5 ng compound A108; B 0.25 ng compound A108). C-D) Quantification of visible lung metastases on the lung surface in response to doses of 2.5 ng and 0.25 ng compound A108 respectively (mean±SEM.; n=12 for control and compound A108, one-way ANOVA). (mean±SEM.; n=11 for control, n=12 for compound A108; one-way ANOVA).

FIG. 10. Various compounds significantly reduce tumour growth and improve the survival of MC38-bearing mice. Tumour weights at various experimental time points in response to A) compound A103 or B) compound A102. C) Kaplan-Meier survival plots of mice s.c. injected with MC38 tumors and treated with 25 ug i.t. of compound A102 on day 7, 9 and 11 (control n=7; compound A102 n=10). Statistic: Log-rank (Mantel-Cox). The number in ( ) indicates the number of tumor free mice.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

There is a need for more reliable and efficacious regimes that have utility in the treatment of cancer. The present inventors have unexpectedly found that when the compounds disclosed herein are administered to a subject with cancer, the treatment is highly effective, including at a number of doses and in a number of models of cancer. Specifically, the administration of a compound disclosed herein provides for one or more of:
significant decreases in tumour growth at multiple doses and in multiple models;
significant increases in survival at multiple doses and in multiple models;
significant reductions in metastasis of tumours; and
almost complete ablation of cancer in response to the administration of a compound described herein.

These remarkable effects are observed regardless of administration route. The inventors have shown these remarkable effects in a number of different models of cancer with differing aetiology and pathogenesis including:
epithelial mammary carcinoma that has characteristics of triple-negative breast cancer;
metastatic breast cancer;
colon carcinoma;
melanoma; and
fibrosarcoma.

A skilled person would therefore understand the applicability of the invention to any of the other cancers described herein or known in the art.

The inventors also describe herein the utility of a number of different compounds in treating cancer including:
compound A101/compound 1 in models of colon cancer, melanoma and metastatic breast cancer which was able to impair tumour growth, prolong survival and reduce metastasis when administered intraperitoneally, intratumourally or systemically;
compounds A102 and A103 in a model of colon cancer which significantly improved subject survival;
compound A108 in models of colon cancer, breast cancer, melanoma, fibrosarcoma and metastatic breast cancer, which significantly reduced tumour growth, improved survival rates and reduced metastasis when administered intraperitoneally, intratumourally or systemically; and
compound A108 in a model of metastatic breast cancer when administered to the respiratory tract.

A skilled person would therefore understand the applicability of the invention to any of the other compounds described herein.

This effect is surprising because the role of compounds that are capable of stimulating TLR2 signalling in treating cancer has been unclear. In particular, it has been reported that subcutaneous or intraperitoneal administration of Pam2CysSK4 and MALP2 have no anti-tumour activity and to the contrary induce IL-10 and Tregs (Yamazaki et al. PLOS ONE 2011 6(4): e18833). The same group reported that intravenous administration of Pam2CysSK4 promotes myeloid derived suppressor cells (Maruyama et al. 2015 457:445e450). Importantly, it has been suggested that endogenous TLR2/6 agonists derived from cancer cells may enhance metastasis (Kim et al. Nature 2009 457: 102-106). Another study also suggested TLR2 stimulation may promote colorectal cancer cell growth via PI3K/Akt and NFκB signalling pathways (Liu et al. International Immunopharmacology 2018 59:375-383).

Thus, the inventors identify the therapeutic utility of the compounds described herein in treating a number of different types of cancer, at different doses, with various routes of administration.

Compounds

Any of the compounds described herein may have utility in the treatment of cancer including cancer metastasis.

As used herein, reference to any compound described herein also includes a pharmaceutically acceptable salt, solvate, polymorph or prodrug thereof.

The compounds described herein may demonstrate substantial stability in solution. This solution stability may be observed by storing solutions of the compounds under ambient storage conditions (eg at 25° C.) or under accelerated degradation stability (eg at 40° C.) for at least about 14 days.

Additional compounds that are useful in any aspect of the present invention are described below.

In any aspect, the compound may be a compound of formula (I):

A-Y—B (I)
wherein A comprises or consists of a moiety selected from A1 and A2:

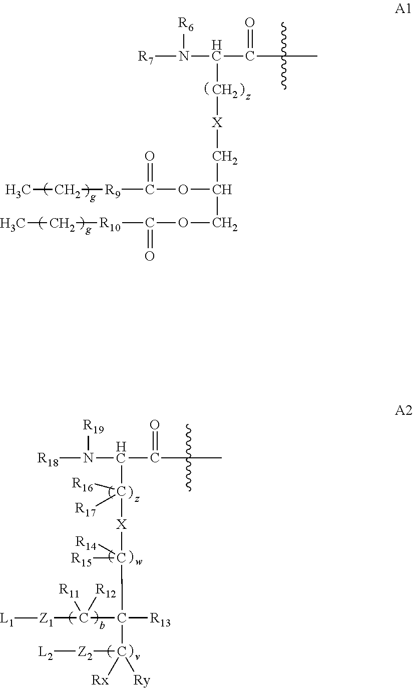

wherein
each z is independently selected from 1 or 2;
each X is independently selected from —S—, —S(═O)— and —S(═O)₂—;
in moiety A1:
   each g is independently 10, 11, 12, 13, 14, 15, 16, 17 or 18;
   R₆ and R₇ are independently selected from the group consisting of H, a straight or branched C₁-C₄ alkyl, and —C(═O)CH;
   R₉ and R₁₀ are independently selected from the group consisting of —NH—, —O— or a single bond; and
in moiety A2:
   b and w are each independently an integer from 0 to 7 and v is an integer from 0 to 5, such as from 2 to 5, provided that:
      the sum of b, v, and w is at least 3; and
      the sum of b and w is from 0 to 7;
   Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
   R₁₁, R₁₂, Rₓ, R_{y}, R₁₄, R₁₅, R₁₆, and R₁₇ are each independently H or C₁-C₆ aliphatic;
   R, R₁₃ and R₁₈ are each independently H or C₁-C₆ aliphatic;
   R₁₉ is H, C₁-C₆ aliphatic, an amino protecting group, L₃-C(═O)—, or A₂;
   L₁ and L₂ are each independently C₅-C₂₁ aliphatic or C₄-C₂₀ heteroaliphatic;
   L₃ is C₁-C₂₁ aliphatic or C₂-C₂₀ heteroaliphatic;
   A₂ is an amino acid or a peptide;
   wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
Y is

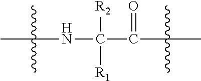

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
and
B comprises or consists of Polyethylene Glycol (PEG),
or a pharmaceutically acceptable salt, solvate or prodrug thereof.

In any aspect, the compound may be a compound of formula (IA1):

A-Y—B (IA1)
wherein A comprises or consists of moiety A1:

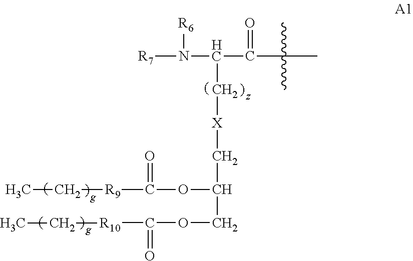

wherein each g is independently 10, 11, 12, 13, 14, 15, 16, 17 or 18;
z is 1 or 2;
X is selected from —S—, —S(═O)— and —S(═O)₂—;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond;
Y is

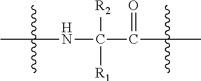

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
and
B comprises or consists of Polyethylene Glycol (PEG),
or a pharmaceutically acceptable salt, solvate or prodrug thereof.

In some embodiments, g is an integer from 12 to 16.

In some embodiments, g is 14.

In any aspect, the compound may be a compound of formula (IA2):

A-Y—B (IA2)
wherein A comprises or consists of:

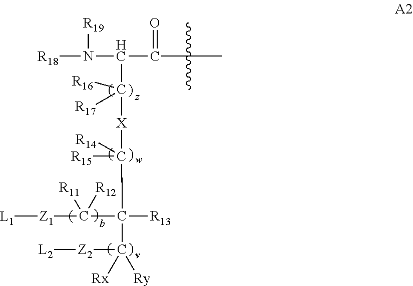

wherein
b and w are each independently an integer from 0 to 7 and v is an integer from 0 to 5, such as from 2 to 5, provided that:
   the sum of b, v, and w is at least 3; and
   the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, S(═O),
—S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
Y is

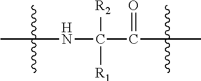

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
and
B comprises or consists of Polyethylene Glycol (PEG),
or a pharmaceutically acceptable salt, solvate or prodrug thereof.

In some embodiments, v is an integer selected from 2, 3, 4 or 5. In some embodiments, v is 2 or 3. In some embodiments, v is 2.

In some embodiments, Rₓ, R_{y}, R₁₁, R₁₂, R₁₃, R₁₄, R₁₅, R₁₆, and R₁₇ are H.

In some embodiments, R and R₁₃ are each H.

In some embodiments, Z₁ and Z₂ are the same and selected from the group consisting of —O—, —NR—, —S—, S(═O), S(═O)₂—, —C(═O)O—, —OC(═O)—, —C(═O)NR—, —NRC(═O)—, —C(═O)S—, —SC(═O)—, OC(═O)O—, NRC(═O)O—, —OC(═O)NR—, and —NRC(═O)NR—.

In some embodiments, Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—.

In some embodiments, w is an integer selected from 1-7. In some embodiments, w is 1.

In some embodiments, b is 0.

In some embodiments, the sum of b and w is from 1 to 7. In these embodiments, b may be an integer selected from 0-7 and w may be an integer selected from 1-7, preferably 1.

In some embodiments, b is 0, w is 1 and v is 2.

In some embodiments, R₁₈ is H.

In some embodiments, R₁₉ is selected from the group consisting of H, C₁-C₆ alkyl, —C(═O) C₁-C₆ alkyl or —C(═O)C₁₁-C₁₉alkyl.

In some embodiments, R₁₉ is selected from H, C₁-C₆ alkyl, —C(═O) C₁-C₆ alkyl, preferably H, C₁-C₄ alkyl, —C(═O) C₁-C₄ alkyl.

In some embodiments, R₁₉ is selected from H and —C(═O)CH₃.

In some embodiments, L₁ and L₂ are independently selected from C₅-C₂₁ aliphatic or C₄-C₂₀ heteroaliphatic. In some embodiments, L₁ and L₂ and independently selected from C₁₀-C₁₈ aliphatic or C₁₀-C₁₈ heteroaliphatic. In some embodiments, L₁ and L₂ are independently selected from C₁₄-alkyl and C₁₅-alkyl.

In some embodiments, X is S.

In some embodiments, X is S(═O).

In some embodiments, X is S(═O)₂.

In some embodiments, R₆ and R₇ are each H.

In some embodiments, R₁₈ and R₁₉ are each H.

In some embodiments, the invention provides a compound of formula (I) wherein:
v is an integer selected from 2 to 5;
b is 0;
Rₓ, R_{y}, R₁₃, R₁₄, R₁₅, R₁₆, and R₁₇ are H;
Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
w is an integer selected from 1 to 7;
R₁₉ is selected from the group consisting of H, C₁-C₆ alkyl, —C(═O) C₁-C₆ alkyl or —C(═O)C₁₁-C₁₉alkyl; and
L₁ and L₂ and independently selected from C₁₀-C₁₆ aliphatic or C₁₀-C₁₈ heteroaliphatic.

In some embodiments, the invention provides a compound wherein
v is 2;
b is 0;
w is 1;
the sum of v, b and w is 3;
the sum of b and w is 1;
z is 1;
X is S
Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each H;
R and R₁₃ are each H;
R₁₈ is H;
R₁₉ is selected from the group consisting of H, C₁-C₆ alkyl, —C(═O) C₁-C₆ alkyl or —C(═O)C₁-C₁₉alkyl; and
L₁ and L₂ and independently selected from C₁₀-C₁₈ aliphatic or C₁₀-C₁₈ heteroaliphatic.

It will be appreciated that any embodiment of a substituent described herein, including substituents R₁, R₂, R₄, R₅, R₆, R₇, R₉, R₁₀, z, X, g, R₁₁, R₁₂, R₁₃, R₁₄, R₁₅, R₁₆, R₁₇, R₁₈, R₁₉, Rₓ, R_{y}, L₁, L₂, Z₁, Z₂, b, v, w, n, m, p, q, R₃, L, t, k and h, is intended to apply to any instance of that substituent for any compound described herein, including compounds of formulas (I)-(XIX).

In any aspect, the compound may be a compound of formula (II):

A-Y′—B (II)
wherein A comprises or consists of moiety A1 or A2 as defined herein;
Y′ is

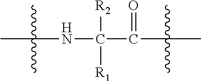

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
and
B comprises or consists of Polyethylene Glycol (PEG),
or a pharmaceutically acceptable salt, solvate or prodrug thereof.

In some embodiments, the compound comprises moiety A1, wherein:
each g is independently 10, 11, 12, 13, 14, 15, 16, 17 or 18;
z is 1;
X is S;
R₆ and R₇ are H;
R₉ and R₁₀ are both a single bond.

In some embodiments, moiety A1 is defined by moiety A1′

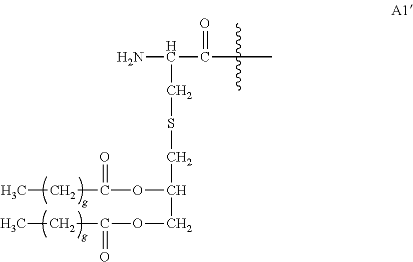

wherein each g is independently 10, 11, 12, 13, 14, 15, 16, 17 or 18.

In any aspect, any of the compounds described herein may be a compound comprising a moiety A selected from A1′ and A2 as defined herein and PEG, wherein the moiety A and PEG are linked by a glycine, serine, homoserine, threonine, phosphoserine, asparagine or glutamine residue, or an ester of a glutamine residue.

In any aspect, the compound may comprise or consist of partial structure A1Y′ or A2Y′:

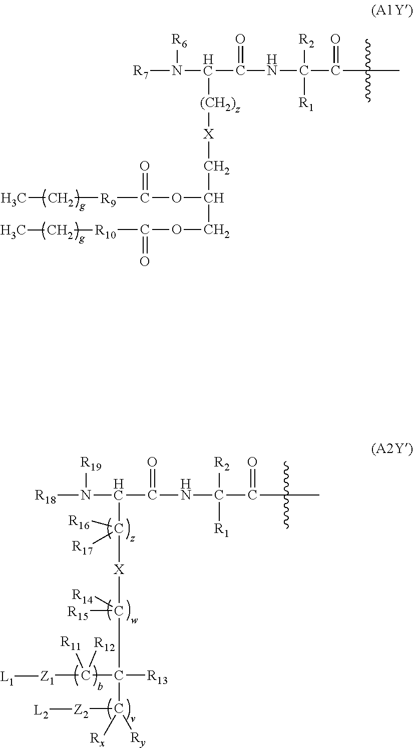

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
b and w are each independently an integer from 0 to 7 and v is an integer from 0 to 5, provided that:
the sum of b, v, and w is at least 3; and
the sum of b and w is from 0 to 7;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independentlyC₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and
A1Y′ or A2Y′ is covalently linked to polyethylene glycol (PEG),
or a pharmaceutically acceptable salt, solvate or prodrug thereof.

In some embodiments, the moiety A and PEG are linked by a serine, homoserine, threonine or phosphoserine residue.

In some embodiments, moiety A and PEG are covalently linked to the glycine, serine, homoserine, threonine, phosphoserine, asparagine or glutamine residue, or an ester of a glutamine residue, through the bond(s) denoted by [symbol] .

In any aspect, the compound may be:

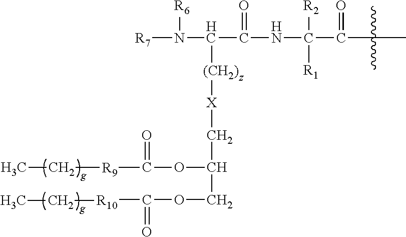

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2; and
X is selected from —S—, —S(═O)— and —S(═O)₂—;
covalently linked to polyethylene glycol (PEG),
or a pharmaceutically acceptable salt, solvate or prodrug thereof.

In some embodiments, the compound may be:

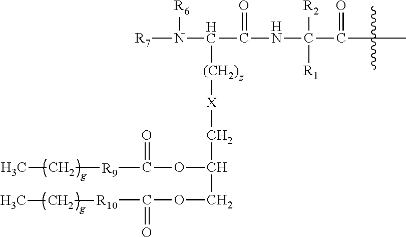

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
   R₆ and R₇ are independently selected from the group consisting of H, a straight or branched C₁-C₄ alkyl, and —C(═O)CH₃;
   R₉ and R₁₀ are independently selected from the group consisting of —NH—, —O— or a single bond;
   z is 1 or 2; and
   X is selected from —S—, —S(═O)— and —S(═O)₂—;
   covalently linked to polyethylene glycol (PEG),
   or a pharmaceutically acceptable salt, solvate or prodrug thereof.

In some embodiments, the compound may be:

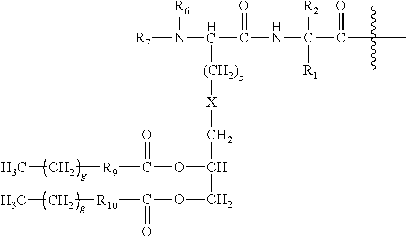

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
   R₆ and R₇ are H;
R₈ is selected from the group consisting of H and a straight or branched C₁-C₆ alkyl;
   R₉ and R₁₀ are both a single bond;
   z is 1; and
   X is S;
   covalently linked to polyethylene glycol (PEG),
   or a pharmaceutically acceptable salt, solvate or prodrug thereof.

In some embodiments, the PEG is covalently linked through the bond denoted by [symbol] .

In some embodiments, the compound may be:

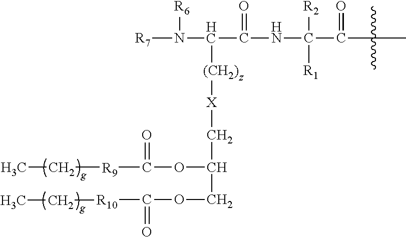

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
   R₆ and R₇ are H;
   R₉ and R₁₀ are both a single bond;
   z is 1; and
   X is S;
   covalently linked to polyethylene glycol (PEG),
   or a pharmaceutically acceptable salt, solvate or prodrug thereof.

In some embodiments, the PEG is covalently linked through the bond denoted by [symbol] .

In some embodiments, the compound may be:

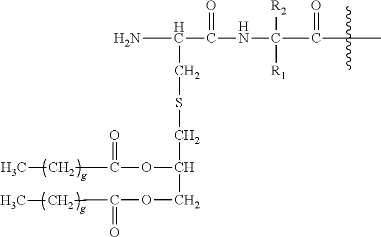

wherein R₁, R₂ and g are as defined herein, salt, solvate or prodrug thereof

In some embodiments, the PEG is covalently linked through the bond denoted by [symbol] .

In some embodiments, the compound may be:

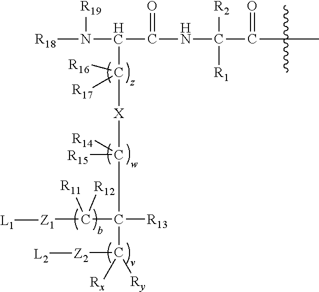

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
b and w are each independently an integer from 0 to 7 and v is an integer from 0 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independentlyC₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
covalently linked to polyethylene glycol (PEG),
or a pharmaceutically acceptable salt, solvate or prodrug thereof.

In some embodiments, the PEG is covalently linked through the bond denoted by [symbol] .

In any aspect, the compound may be a compound of formula (III):

AY-B (III)
wherein
AY comprises or consists of a moiety selected from AY1 and AY2

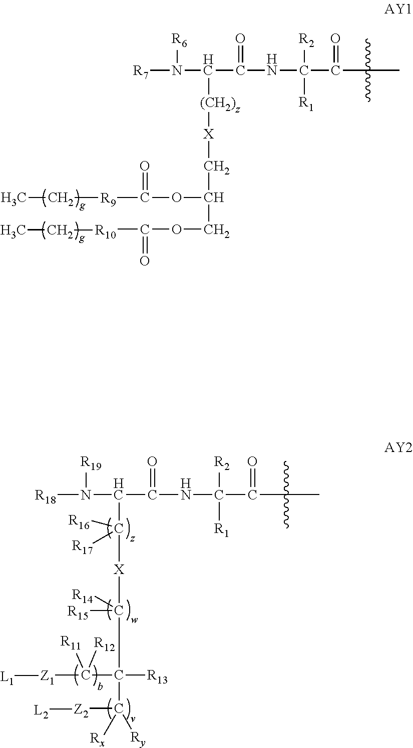

wherein each of R₁, R₂, R₆, R₇, R₉, R₁₀, z, X, g, R₁₁, R₁₂, R₁₃, R₁₄, R₁₅, R₁₆, R₁₇, R₁₈, R₁₉, Rₓ, R_{y}, L₁, L₂, Z₁, Z₂, b, v and w are as defined for the compound of formula (I); and
B comprises or consists of Polyethylene Glycol (PEG).

In any aspect, the compound may be a compound of formula (IV):

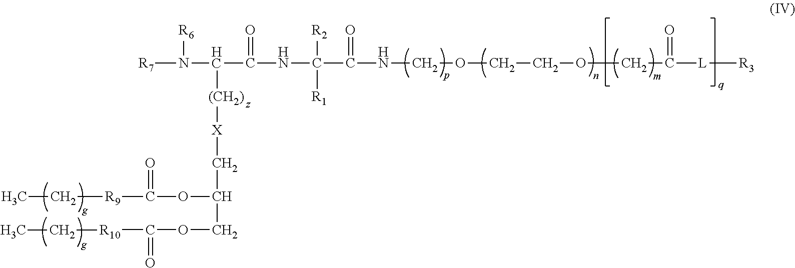

wherein
n is 3 to 100;
m is 1, 2, 3 or 4;
each g is independently 10, 11, 12, 13, 14, 15, 16, 17 or 18;
p is 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

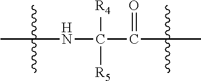

wherein R₄ is H; and
R₅ is the side chain, or second hydrogen of the amino acid
or a pharmaceutically acceptable salt, solvate or prodrug thereof.

In any aspect, the compound may be a compound of formula (V):

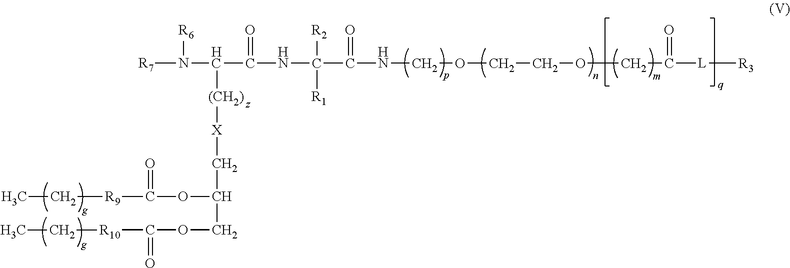

wherein
n is 3 to 100;
m is 1, 2, 3 or 4;
each g is independently 10, 11, 12, 13, 14, 15, 16, 17 or 18;
p is 2, 3 or 4;
q is null or 1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

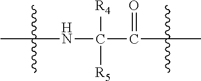

wherein R₄ is H; and
R₅ is the side chain, or second hydrogen of the amino acid
or a pharmaceutically acceptable salt, solvate or prodrug thereof.

In some embodiments, the compound is a compound of formula (IV) or (V) wherein R₆ and R₇ are H;
R₈ is selected from the group consisting of H and a straight or branched C₁-C₆ alkyl;
R₉ and R₁₀ are both a single bond;
z is 1; and
X is S.

In some embodiments, the compound of any one of formulas (I)-(V) may be a compound of formula (VI):

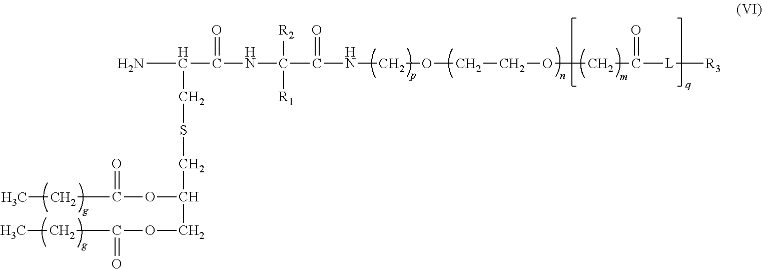

wherein
n is 3 to 100;
m is 1, 2, 3 or 4;
each g is independently 10, 11, 12, 13, 14, 15, 16, 17 or 18;
p is 2, 3 or 4;
q is null or 1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

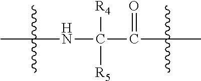

wherein R₄ is H; and
R₅ is the side chain, or second hydrogen of the amino acid
or a pharmaceutically acceptable salt, solvate or prodrug thereof.

In any aspect, the compound may be a compound of formula (VII):

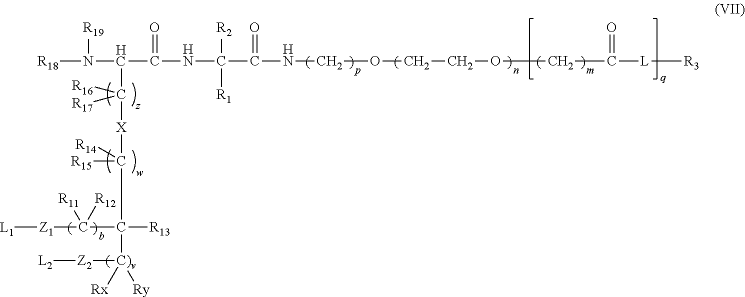

wherein
n is 3 to 100;
m is 1, 2, 3 or 4;
p is 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and CH₂CH₂C(═O)OR₈, wherein any one of the alkyl hydrogens can be replaced with a halogen;
R₈ is selected from the group consisting of H and a straight or branched C₁-C₆ alkyl;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

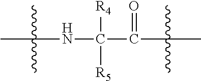

wherein R₄ is H; and
R₅ is the side chain, or second hydrogen of the amino acid;
b and w are each independently an integer from 0 to 7 and v is an integer from 0 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
or a pharmaceutically acceptable salt, solvate or prodrug thereof.

In any aspect, the compound may be a compound of formula (VIII):

A-Y—NH—(CH₂)ₚ—O—(CH₂—CH₂—O)ₙ—[(CH₂)ₘ—CO-L-]_{q}R₃ (VIII)
wherein
A is a moiety selected from A1 and A2 as defined herein
Y is

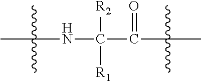

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
n is 3 to 100;
m is 1, 2, 3 or 4;
each g is independently 10, 11, 12, 13, 14, 15, 16, 17 or 18;
p is 2, 3 or 4;
q is null or 1;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

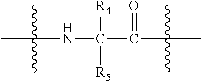

wherein R₄ is H; and
R₅ is the side chain, or second hydrogen of the amino acid,
or a pharmaceutically acceptable salt, solvate or prodrug thereof.

In any aspect, the compound may be a compound of formula (IX):

A1-Y—NH—(CH₂)ₚ—O—(CH₂—CH₂—O)ₙ—[(CH₂)ₘ—CO-L-]_{q}R₃ (IX)
wherein
A1 is represented by moiety A1 as defined for formula (I)
Y is

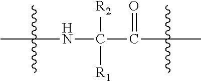

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond;
z is 1 or 2;
X is selected from —S—, —S(═O)— and —S(═O)₂—;
n is 3 to 100;
m is 1, 2, 3 or 4;
each g is independently 10, 11, 12, 13, 14, 15, 16, 17 or 18;
p is 2, 3 or 4;
q is null or 1;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

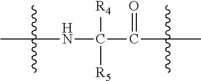

wherein R₄ is H; and
R₅ is the side chain, or second hydrogen of the amino acid,
or a pharmaceutically acceptable salt, solvate or prodrug thereof.

In some embodiments, the compound is a compound of formula (VIII) or (IX), wherein
R₆ and R₇ are H;
R₉ and R₁₀ are both a single bond;
z is 1;
X is S;
salt, solvate or prodrug thereof salt, solvate or prodrug thereof salt, solvate or prodrug thereof In any aspect, the compound may be a compound of formula (X):

Pam2Cys-Y—NH—(CH₂)ₚ—O—(CH₂—CH₂—O)ₙ—[(CH₂)ₘ—CO-L-]_{q}R₃ (X)
wherein
Pam2Cys has the structure:

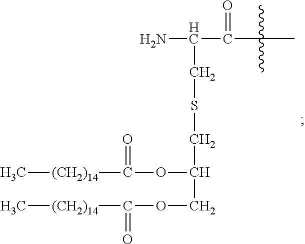

Y is:

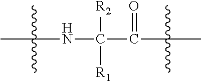

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
n is 3 to 100;
m is 1, 2, 3 or 4;
p is 2, 3 or 4;
q is null or 1;
wherein when q=1, R₃ is H, —NH₂ or —OH;
wherein when q=0, R₃ is H;
L is null or consists of 1 to 10 units, wherein each unit is a natural alpha amino acid or derived from a natural alpha amino acid, and has the formula:

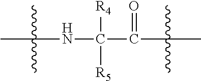

wherein R₄ is H; and
R₅ is the side chain, or second hydrogen of the amino acid,
or a pharmaceutically acceptable salt, solvate or prodrug thereof.

In any aspect, the compound may be a compound of formula (XI):

Pam2Cys-Y—NH—(CH₂)ₚ—O—(CH₂—CH₂—O)ₙ—[(CH₂)ₘ—CO-L-]_{q}R₃ (XI)
wherein
Pam2Cys has the structure:

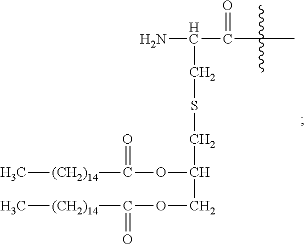

Y is:

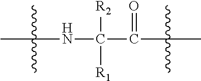

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen and wherein R₁ and R₂ are not both H;
n is 3 to 100;
m is 1, 2, 3 or 4;
p is 2, 3 or 4;
q is null or 1;
wherein when q=1, R₃ is H, —NH₂ or —OH;
wherein when q=0, R₃ is H;
L is null or consists of 1 to 10 units, wherein each unit is a natural alpha amino acid or derived from a natural alpha amino acid, and has the formula:

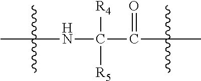

wherein R₄ is H; and
R₅ is the side chain, or second hydrogen of the amino acid,
or a pharmaceutically acceptable salt, solvate or prodrug thereof.

In any aspect, the compound may be a compound of formula (XII):

Pam2Cys-Y—NH—(CH₂)ₚ—O—(CH₂—CH₂—O)ₙ—[(CH₂)ₘ—CO-L-]_{q}R₃ (XII)
wherein
Pam2Cys has the structure:

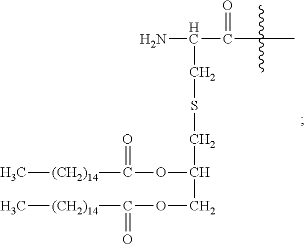

Y is:

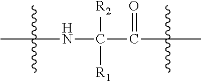

wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
n is 3 to 100;
m is 1, 2, 3 or 4;
p is 2, 3 or 4;
q is null or 1;
wherein when q=1, R₃ is H, —NH₂ or —OH;
wherein when q=0, R₃ is H;
L is null or consists of 1 to 10 units, wherein each unit is a natural alpha amino acid or derived from a natural alpha amino acid, and has the formula:

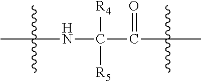

wherein R₄ is H; and
R₅ is the side chain, or second hydrogen of the amino acid,
or a pharmaceutically acceptable salt, solvate or prodrug thereof.

In any aspect, the compound may be a compound of formula (XIII):

Pam2Cys-Ser-NH—(CH₂)ₚ—O—(CH₂—CH₂—O)ₙ—[(CH₂)ₘ—CO-L-]_{q}R₃ (XIII)
wherein
Pam2Cys-Ser has the structure:

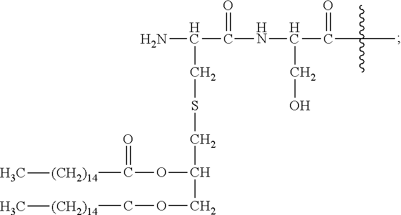

n is 3 to 100;
m is 1, 2, 3 or 4;
p is 2, 3 or 4;
q is null or 1;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

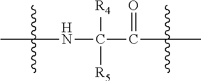

wherein R₄ is H; and
R₅ is the side chain, or second hydrogen of the amino acid,
or a pharmaceutically acceptable salt, solvate or prodrug thereof.

In one embodiment, the compound has the formula (XIV):

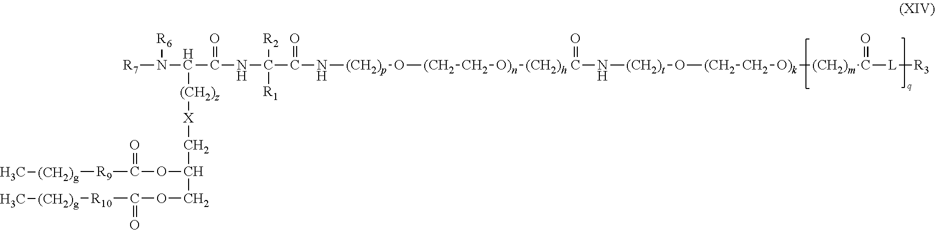

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

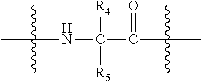

wherein R₄ is H; and
R₈ is the side chain, or second hydrogen of the amino acid,
or a pharmaceutically acceptable salt, solvate or prodrug thereof.

In one embodiment, the compound has the formula (XV):

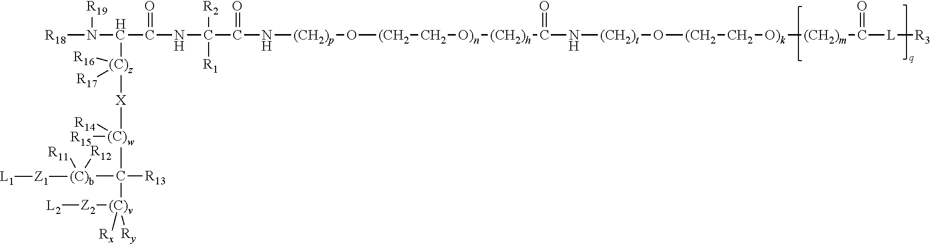

formula (XV)
wherein
n is 3 to 100;
k is 3 to 100;
m is 1, 2, 3 or 4;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

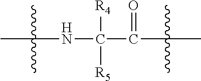

wherein R₄ is H; and
R₅ is the side chain, or second hydrogen of the amino acid;
b and w are each independently an integer from 0 to 7 and v is an integer from 0 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
or a pharmaceutically acceptable salt, solvate or prodrug thereof.

In any aspect, the compound may be a compound of formula (XVI):

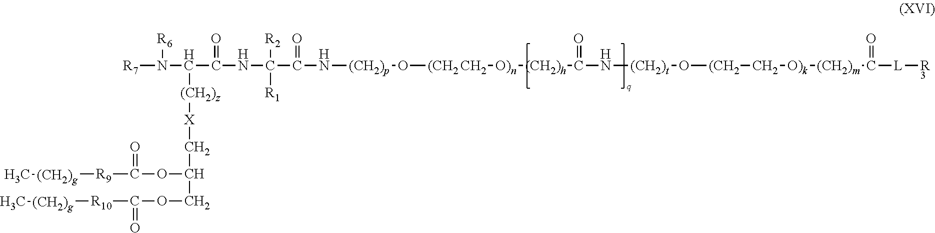

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

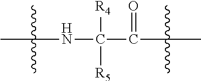

wherein R₄ is H; and
R₅ is the side chain, or second hydrogen of the amino acid,
or a pharmaceutically acceptable salt, solvate or prodrug thereof.

In any aspect, the compound may be a compound of formula (XVII):

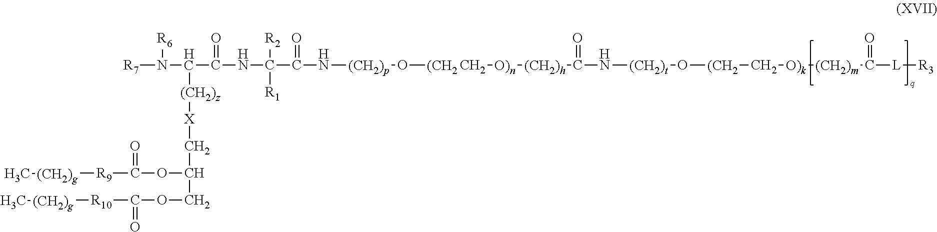

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are H;
R₈ is selected from the group consisting of H and a straight or branched C₁-C₆ alkyl;
R₉ and R₁₀ are both a single bond;
z is 1;
X is S;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

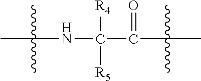

wherein R₄ is H; and
R₅ is the side chain, or second hydrogen of the amino acid,
or a pharmaceutically acceptable salt, solvate or prodrug thereof.

In any aspect, the compound may be a compound of formula (XVIII):

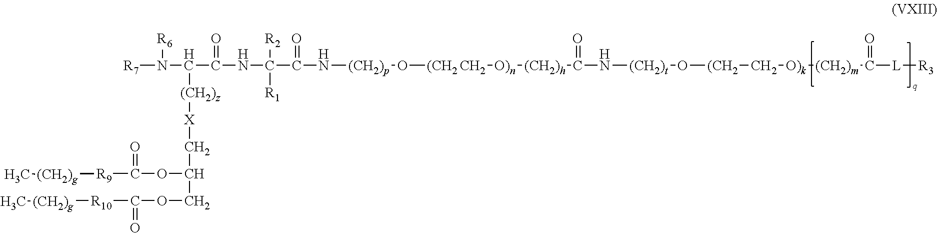

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
wherein R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are H;
R₉ and R₁₀ are both a single bond;
z is 1;
X is S;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

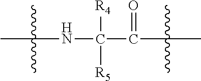

wherein R₄ is H; and
R₅ is the side chain, or second hydrogen of the amino acid,
or a pharmaceutically acceptable salt, solvate or prodrug thereof.

In any aspect, the compound may be a compound of formula (XIX):

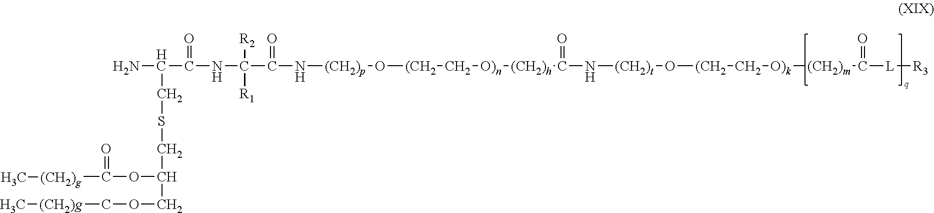

wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula:

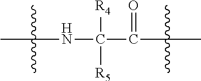

wherein R₄ is H; and
R₅ is the side chain, or second hydrogen of the amino acid,
or a pharmaceutically acceptable salt, solvate or prodrug thereof.

In some embodiments, any compound disclosed herein (including a compound of any one of formulas (I)-(XIX)) that comprises polyethylene glycol (PEG) may comprise the PEG in the form of a substituted PEG.

In some embodiments, the substituted PEG is represented by partial formula B-I:

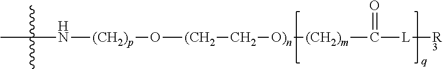

wherein
n is 3 to 100;
m is 1, 2, 3 or 4;
p is 2, 3 or 4;
q is null or 1;
R₃ is H, —NH₂ or —OH, wherein when q is null, R₃ is H and when q is 1, R₃ is —NH₂ or —OH;
L is null or consists of 1 to 10 units, wherein each unit is a natural alpha amino acid or derived from a natural alpha amino acid, and has the formula:

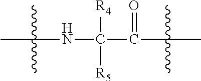

wherein R₄ is H; and
R₅ is the side chain, or second hydrogen of the amino acid.

In some embodiments, the substituted PEG is represented by partial formula B-II:

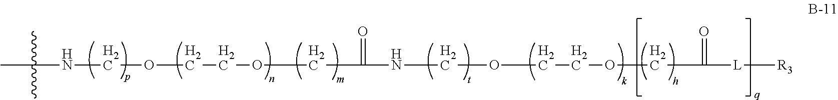

wherein
p is 2, 3 or 4;
n is 3 to 100;
m is 1, 2, 3 or 4;
t is 2, 3 or 4;
k is 3 to 100;
h is 1, 2, 3 or 4;
q is null or 1;
wherein when q is 1, R₃ is —NH₂ or —OH;
wherein when q is null, R₃ is H;
L is null or consists of 1 to 10 units, wherein each unit is a natural alpha amino acid or derived from a natural alpha amino acid, and has the formula:

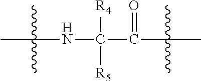

wherein R₄ is H; and
R₅ is the side chain, or second hydrogen of the amino acid.

In some embodiments of the substituted PEG of formula B-I or B-II, q is 1.

In some embodiments of the substituted PEG of formula B-I or B-II, n may be from 10 to 14, such as 11, or from 24 to 30, such as 27.

In some embodiments of the substituted PEG of formula B-I or B-II, m is from 1 to 3, such as 2.

In some embodiments of the substituted PEG of formula B-I or B-II, when q is 1, R₃ is —NH₂.

In some embodiments of the substituted PEG of formula B-I or B-II, L is a natural alpha amino acid residue.

Compounds described herein may exist in and be isolated in optically active and racemic forms. As would be understood by a person skilled in the art, the present invention is intended to encompass any racemic, optically active or stereoisomeric form, or mixtures thereof, of compounds of the invention which possess the useful properties described herein. It is well known in the art how to prepare such forms (for example, by resolution of racemic mixtures by recrystallization, by synthesis from optically-active starting materials, by chiral synthesis, or by chiral chromatographic separation). In some embodiments, a composition may comprise a compound in an enantiomerically or diastereomerically enriched form. For example, the compound may have an enantiomeric excess (ee) or a diastereomeric excess (de) of at least about 1%, 5%, 10%, 20%, 30%, 40%, 50%, 60%, 70%, 80%, 90%, 95%, 96%, 97%, 98%, 99% or more than 99%. In some embodiments, the compound may be enriched by at least about 1%, 5%, 10%, 20%, 30%, 40%, 50%, 60%, 70%, 80%, 90%, 95%, 96%, 97%, 98%, 99% or more than 99% at any stereocentre of the compound.

In any aspect, the compound may comprise a chiral centre around the following chiral centre (shown at *):

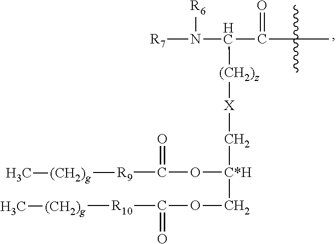

wherein the chiral centre is in the R configuration. A compound in this form may also be referred to as an R-Pam2 analogue diastereomer of a compound of the invention as described herein. This may be depicted as:

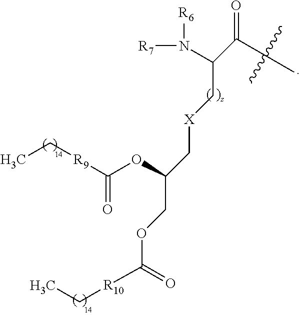

In any aspect, the compound may comprise a chiral centre in the 2,3-bis(palmitoyloxy)propyl moiety of Pam2Cys (shown at *):

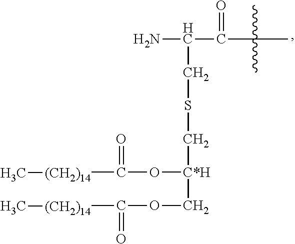

wherein the chiral centre is in the R configuration. A compound in this form may also be referred to as an R-Pam2 diastereomer of a compound of the invention as described herein. This may be depicted as:

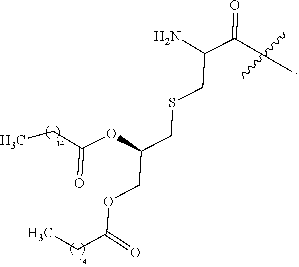 [SMILES: *C(=O)C(N)CSC[C@@H](COC(=O)CC)OC(=O)CC]

In any aspect, the compound may comprise a chiral centre around the following chiral centre (shown at *):

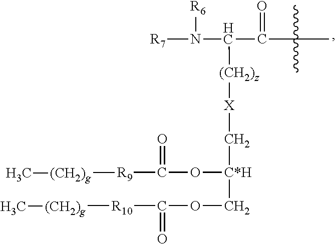

wherein the chiral centre is in the S configuration. A compound in this form may also be referred to as an S-Pam2 analogue diastereomer of a compound of the invention as described herein. This may be depicted as:

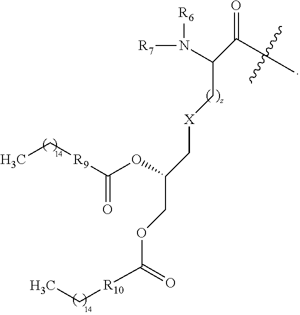

In any aspect, the compound comprises a chiral centre in the 2,3-bis(palmitoyloxy)propyl moiety of Pam2Cys (shown at *):

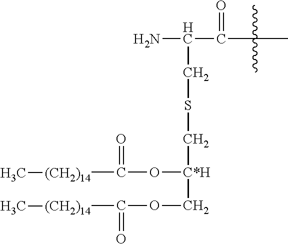

wherein the chiral centre is in the S configuration. A compound in this form may also be referred to as an S-Pam2 diastereomer of a compound of the invention as described herein. This may be depicted as:

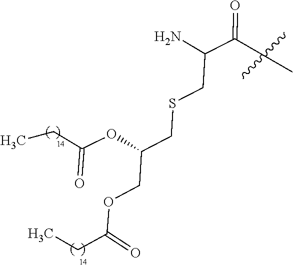 [SMILES: *C(=O)C(N)CSC[C@H](COC(=O)CC)OC(=O)CC]

In any aspect, the compound comprises a chiral centre around the following chiral centre (shown at *):

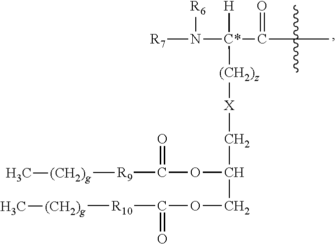

wherein the chiral centre is in the L configuration. A compound in this form may also be referred to as an L-Cys analogue diastereomer of Pam2Cys of a compound of the invention as described herein. This may be depicted as:

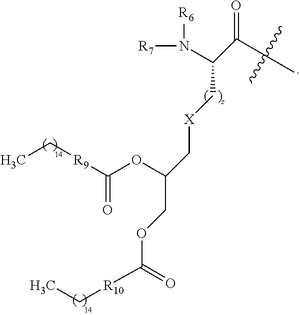

In any aspect, the compound comprises a chiral centre in the cysteine residue of Pam2Cys (shown at *):

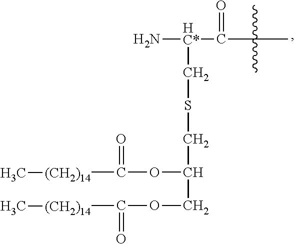

wherein the chiral centre is in the L configuration. A compound in this form may also be referred to as an L-Cys diastereomer of Pam2Cys of a compound of the invention as described herein. This may be depicted as:

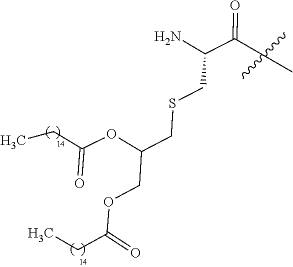 [SMILES: *C(=O)[C@@H](N)CSCC(COC(=O)CC)OC(=O)CC]

Other stereocentres in these compounds may be racemic or independently enriched in either the R or S configuration.

In any aspect, the compound comprises a chiral centre in moiety A1 around the following chiral centre (shown at *):

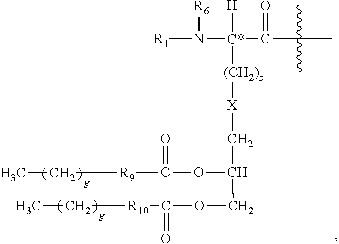

wherein the chiral centre is in the D configuration. A compound in this form may also be referred to as an D-Cys analogue diastereomer of Pam2Cys of a compound described herein.

This may be depicted as:

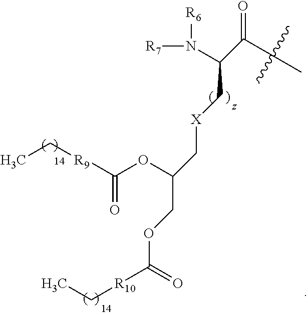

Other stereocentres in these compounds may be racemic or independently enriched in either the R or S configuration.

In any aspect, the compound comprises a chiral centre in the cysteine residue of Pam2Cys (shown at *):

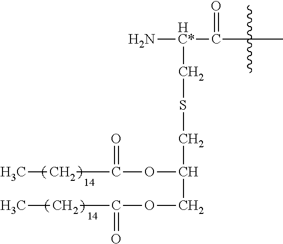

wherein the chiral centre is in the D configuration. A compound in this form may also be referred to as an D-Cys diastereomer of Pam2Cys of a compound described herein. This may be depicted as:

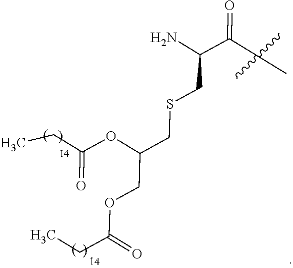 [SMILES: *C(=O)[C@H](N)CSCC(COC(=O)CC)OC(=O)CC]

Other stereocentres in these compounds may be racemic or independently enriched in either the R or S configuration.

In any aspect or embodiment of the invention, a compound of the present invention may be provided in a chiral form enriched at a chiral centre at the following carbon atom (shown at *) of moiety A2:

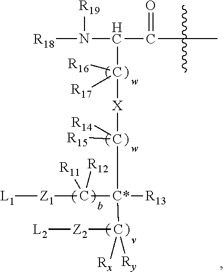

wherein the chiral centre is in the R configuration. In some embodiments, this stereoisomer of the compound may be depicted as:

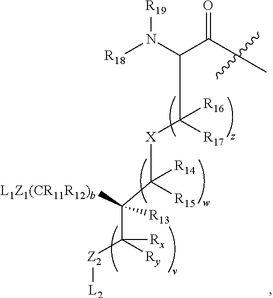

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v and z are as defined for the compound of Formula (I) and w is 1. Other stereocentres in these compounds may be racemic or independently enriched in either the R or S configuration.

In any aspect or embodiment of the invention, a compound of the present invention may be provided in a chiral form enriched at a chiral centre at the following carbon atom (shown at *) of moiety A2:

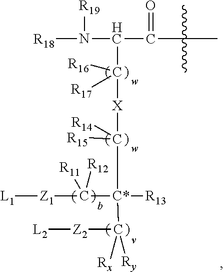

wherein the chiral centre is in the S configuration. In some embodiments, moiety A of this stereoisomer of the compound may be depicted as:

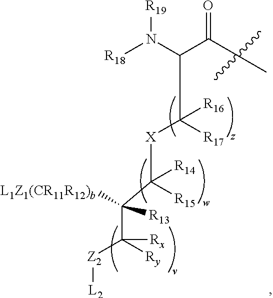

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v, w, and z are as defined for the compound or Formula (I). Other stereocentres in these compounds may be racemic or independently enriched in either the R or S configuration.

In any aspect or embodiment of the invention, a compound of the present invention may be provided in a chiral form enriched at a chiral centre at the following carbon atom (shown at **) of moiety A2:

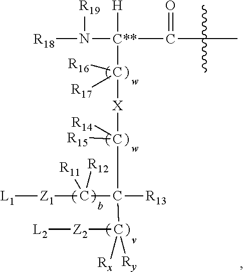

wherein the chiral centre is in the L configuration. A compound in this form may also be referred to as an L-Cys analogue stereoisomer of a compound of the invention. In some embodiments, this stereoisomer of the compound may be depicted as:

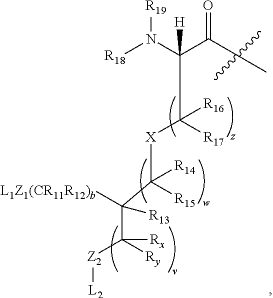

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v, w, and z are as defined for the compound or Formula (I). Other stereocentres in these compounds may be racemic or independently enriched in either the R or S configuration.

In any aspect or embodiment of the invention, a compound of the present invention may be provided in a chiral form enriched at a chiral centre at the following carbon atom (shown at **) of moiety A2:

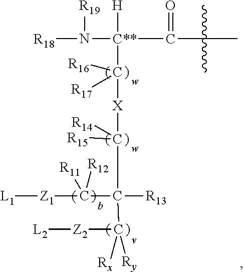

wherein the chiral centre is in the D configuration. A compound in this form may also be referred to as a D-Cys analogue stereoisomer of a compound of the invention. In some embodiments, moiety A of this stereoisomer of the compound may be depicted as:

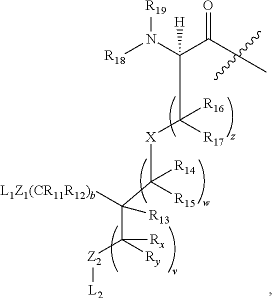

wherein L₁, L₂, Z₁, Z₂, Rₓ, R_{y}, R₁₁, R₁₂, R₁₃, R₁₄, R₁₅, R₁₆, R₁₇, R₁₈, R₁₉, b, v and z are as defined for the compound or Formula (I) and w is 1. Other stereocentres in these compounds may be racemic or independently enriched in either the R or S configuration.

In any aspect, the compound comprises a chiral centre in the Y moiety of the compound (shown at *):

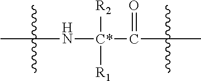

wherein the chiral centre is in the L-configuration. A compound in this form may also be referred to as an L-Y diastereomer of a compound described herein.

In any aspect, the compound comprises a chiral centre in the Y moiety of the compound (shown at *):

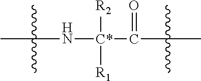

wherein the chiral centre is in the D-configuration. A compound in this form may also be referred to as a D-Y diastereomer of a compound described herein.

In any aspect, compositions comprising a compound of the invention (including a compound of any one of formulas (I)-(XIX)) or a pharmaceutically acceptable salt, solvate or prodrug thereof, and a pharmaceutically acceptable carrier, diluent or excipient may be used in a method or use of the invention.

In some embodiments, the compound as described herein is the R diastereomer around the chiral centre of the 2,3-bis(palmitoyloxy)propyl moiety of the compound.

In some embodiments, the compound as described herein is the S diastereomer around the chiral centre of the 2,3-bis(palmitoyloxy)propyl moiety of the compound.

In any aspect, a composition as described herein comprises a compound that is the R diastereomer around the chiral centre of the 2,3-bis(palmitoyloxy)propyl moiety of the compound.

In any aspect, a composition comprises a compound that is the S diastereomer around the chiral centre of the 2,3-bis(palmitoyloxy)propyl moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in a composition is the R diastereomer around the chiral centre of the 2,3-bis(palmitoyloxy)propyl moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in a composition is the S diastereomer around the chiral centre of the 2,3-bis(palmitoyloxy)propyl moiety of the compound (for example moiety A1).

In any aspect, the compound as described herein is the L diastereomer around the chiral centre of the cysteine analogue residue of the Pam2Cys analogue moiety compound (for example moiety Y).

In any aspect, the compound as described herein is the L diastereomer around the chiral centre of the cysteine residue of the Pam2Cys moiety compound (for example moiety Y).

In any aspect, the compound as described herein is the D diastereomer around the chiral centre of the cysteine analogue residue of the Pam2Cys analogue moiety compound (for example moiety Y).

In any aspect, the compound as described herein is the D diastereomer around the chiral centre of the cysteine residue of the Pam2Cys moiety of the compound (for example moiety Y).

In any aspect, a composition as described herein comprises a compound that is the L diastereomer around the chiral centre of the cysteine analogue residue of the Pam2Cys analogue moiety of the compound (for example moiety Y).

In any aspect, a composition as described herein comprises a compound that is the L diastereomer around the chiral centre of the cysteine residue of the Pam2Cys moiety of the compound (for example moiety Y).

In any aspect, a composition as described herein comprises a compound that is the D diastereomer around the chiral centre of the cysteine analogue residue of the Pam2Cys analogue moiety of the compound (for example moiety Y).

In any aspect, a composition as described herein comprises a compound that is the D diastereomer around the chiral centre of the cysteine residue of the Pam2Cys moiety of the compound (for example moiety Y).

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine analogue residue of the Pam2Cys analogue moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the cysteine residue of the Pam2Cys moiety of the compound.

In any aspect 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine analogue residue of the Pam2Cys analogue moiety of the compound.

In any aspect, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the cysteine residue of the Pam2Cys moiety of the compound.

In any aspect, the compound of the invention as described herein is the L diastereomer around the chiral centre of the Y moiety.

In any aspect, the compound as described herein is the D diastereomer around the chiral centre of the Y moiety.

In any aspect, a composition as described herein comprises a compound that is the L diastereomer around the chiral centre of the Y moiety.

In any aspect, a composition as described herein comprises a compound that is the D diastereomer around the chiral centre of the Y moiety.

In any aspect, 1%, 5%, 10%, 20%, 30%, 40%, 50%, 60%, 70%, 80%, 90%, 95%, 96%, 97%, 98%, 99% or more than 99% of the compound present in the composition is the L diastereomer around the chiral centre of the Y moiety.

In any aspect, 1%, 5%, 10%, 20%, 30%, 40%, 50%, 60%, 70%, 80%, 90%, 95%, 96%, 97%, 98%, 99% or more than 99% of the compound present in the composition is the D diastereomer around the chiral centre of the Y moiety.

The compounds of formulas (I)-(XIX) described herein may demonstrate substantial stability in solution. This solution stability may be observed by storing solutions of the compounds under ambient storage conditions (eg at 25° C.) or under accelerated degradation stability (eg at 40° C.) for at least about 14 days.

In any aspect, any of the compounds described herein may be administered in the form of a pharmaceutically acceptable salt.

The term “pharmaceutically acceptable” may be used to describe any pharmaceutically acceptable salt, hydrate or prodrug, or any other compound which upon administration to a subject, is capable of providing (directly or indirectly) a compound of the invention as described herein, or a pharmaceutically acceptable salt, prodrug or ester thereof, or an active metabolite or residue thereof.

Suitable pharmaceutically acceptable salts may include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts may include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of the invention as described herein, such as anhydrous forms, hydrous forms, solvate forms and mixed solvate forms.

Compounds of the invention described herein are intended to cover, where applicable, solvated as well as unsolvated forms of the compounds. Thus compounds of the invention described herein include compounds having the indicated structures, including the hydrated or solvated forms, as well as the non-hydrated and non-solvated forms.

As used herein, the term “solvate” refers to a complex of variable stoichiometry formed by a solute (in this invention, a compound of the invention described herein, or a pharmaceutically acceptable salt, prodrug or ester thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds as described herein are to also include isotope variations, such as the replacement of hydrogen for deuterium.

A “prodrug” is a compound that may not fully satisfy the structural requirements of the compounds provided herein, but is modified in vivo, following administration to a subject or patient, to produce a compound of the invention as described herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Prodrugs include compounds wherein an amino acid residue, or a polypeptide chain of two or more (eg, two, three or four) amino acid residues which are covalently joined to free amino, and amido groups of any of compounds of Formulas (I)-(XIX). The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methionine sulfone. Prodrugs also include compounds wherein carbonates, carbamates, amides and alkyl esters which are covalently bonded to the above substituents of the compounds described herein, including the compounds of formulas (I)-(XIX), or other structure as depicted herein.

The general chemical terms used in the formulae herein have their usual meaning.

The term “aliphatic” is intended to include saturated and unsaturated, nonaromatic, straight chain, branched, acyclic, and cyclic hydrocarbons. Those skilled in the art will appreciate that aliphatic groups include, for example, alkyl, alkenyl, alkynyl, cycloalkyl, and cycloalkenyl groups, and hybrids thereof such as (cycloalkyl)alkyl, (cycloalkenyl)alkyl and (cycloalkyl)alkenyl groups. In various embodiments, aliphatic groups comprise from 1-12, 1-8, 1-6, or 1-4 carbon atoms. In some embodiments, aliphatic groups comprise 5-21, from 9-21, or from 11-21 carbon atoms, such as from 11, 13, 15, 17, or 19 carbon atoms. In some embodiments, the aliphatic group is saturated.

The term “heteroaliphatic” is intended to include aliphatic groups, wherein one or more chain and/or ring carbon atoms are independently replaced with a heteroatom, preferably a heteroatom selected from oxygen, nitrogen and sulfur. In some embodiments, the heteroaliphatic is saturated. Examples of heteroaliphatic groups include linear or branched, heteroalkyl, heteroalkenyl, and heteroalkynyl groups.

The term “alkyl” is intended to include saturated straight chain and branched chain hydrocarbon groups. In some embodiments, alkyl groups have from 1 to 12, 1 to 10, 1 to 8, 1 to 6, or from 1 to 4 carbon atoms. In some embodiments, alkyl groups have from 5-21, from 9-21, or from 11-21 carbon atoms, such as from 11, 13, 15, 17, or 19 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “alkenyl” is intended to include straight and branched chain alkyl groups having at least one double bond between two carbon atoms. In some embodiments, alkenyl groups have from 2 to 12, from 2 to 10, from 2 to 8, from 2 to 6, or from 2 to 4 carbon atoms. In some embodiments, alkenyl groups have from 5-21, from 9-21, or from 11-21 carbon atoms, such as from 11, 13, 15, 17, or 19 carbon atoms. In some embodiments, alkenyl groups have one, two, or three carbon-carbon double bonds. Examples of alkenyl groups include, but are not limited to, vinyl, allyl, —CH═CH(CH₃), —CH═C(CH₃)₂, —C(CH₃)═CH₂, and —C(CH₃)═CH(CH₃).

The term “alkynyl” is intended to include straight and branched chain alkyl groups having at least one triple bond between two carbon atoms. In some embodiments, the alkynyl group have from 2 to 12, from 2 to 10, from 2 to 8, from 2 to 6, or from 2 to 4 carbon atoms. In some embodiments, alkynyl groups have one, two, or three carbon-carbon triple bonds. Examples include, but are not limited to, —C═CH, —C═CH₃, —CH₂C═CH₃, and —C═CH₂CH(CH₂CH₃)₂.

The term “heteroalkyl” is intended to include alkyl groups, wherein one or more chain carbon atoms are replaced with a heteroatom, preferably a heteroatom selected from the group consisting of oxygen, nitrogen, and sulfur. In some embodiments, the heteroalkyl is saturated. Heteroalkyl groups include, for example, polyethylene glycol groups and polyethylene glycol ether groups, and the like.

The term “cycloalkyl” is intended to include mono-, bi- or tricyclic alkyl groups. In some embodiments, cycloalkyl groups have from 3 to 12, from 3 to 10, from 3 to 8, from 3 to 6, from 3 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. In some embodiments, the cycloalkyl group has from 3 to 8, from 3 to 7, from 3 to 6, from 4 to 6, from 3 to 5, or from 4 to 5 ring carbon atoms. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “cycloalkenyl” is intended to include non-aromatic cycloalkyl groups having at least one double bond between two carbon atoms. In some embodiments, cycloalkenyl groups have one, two or three double bonds. In some embodiments, cycloalkenyl groups have from 4 to 14, from 5 to 14, from 5 to 10, from 5 to 8, or from 5 to 6 carbon atoms in the ring(s). In some embodiments, cycloalkenyl groups have 5, 6, 7, or 8 ring carbon atoms. Examples of cycloalkenyl groups include cyclohexenyl, cyclopentenyl, cyclohexadienyl, butadienyl, pentadienyl, and hexadienyl.

The term “aryl” is intended to include cyclic aromatic hydrocarbon groups that do not contain any ring heteroatoms. Aryl groups include monocyclic, bicyclic and tricyclic ring systems. Examples of aryl groups include, but are not limited to, phenyl, azulenyl, heptalenyl, biphenyl, fluorenyl, phenanthrenyl, anthracenyl, indenyl, indanyl, pentalenyl, and naphthyl. In some embodiments, aryl groups have from 6 to 14, from 6 to 12, or from 6 to 10 carbon atoms in the ring(s). In some embodiments, the aryl groups are phenyl or naphthyl. Aryl groups include aromatic-aliphatic fused ring systems. Examples include, but are not limited to, indanyl and tetrahydronaphthyl.

The term “heterocyclyl” is intended to include non-aromatic ring systems containing 3 or more ring atoms, of which one or more is a heteroatom. In some embodiments, the heteroatom is nitrogen, oxygen, or sulfur. In some embodiments, the heterocyclyl group contains one, two, three, or four heteroatoms. In some embodiments, heterocyclyl groups include mono-, bi- and tricyclic rings having from 3 to 16, from 3 to 14, from 3 to 12, from 3 to 10, from 3 to 8, or from 3 to 6 ring atoms. Heterocyclyl groups include partially unsaturated and saturated ring systems, for example, imidazolinyl and imidazolidinyl. Heterocyclyl groups include fused and bridged ring systems containing a heteroatom, for example, quinuclidyl. Heterocyclyl groups include, but are not limited to, aziridinyl, azetidinyl, azepanyl, diazepanyl, 1,3-dioxanyl, 1,3-dioxolanyl, isoxazolidinyl, morpholinyl, piperazinyl, piperidinyl, pyranyl, pyrazolidinyl, pyrrolinyl, pyrrolidinyl, tetrahydrofuranyl, tetrahydrothienyl, thiadiazolidinyl, and trithianyl.

The term “heteroaryl” is intended to include aromatic ring systems containing 5 or more ring atoms, of which, one or more is a heteroatom. In some embodiments, the heteroatom is nitrogen, oxygen, or sulfur. In some embodiments, heteroaryl groups include mono-, bi- and tricyclic ring systems having from 5 to 16, from 5 to 14, from 5 to 12, from 5 to 10, from 5 to 8, or from 5 to 6 ring atoms. Heteroaryl groups include, but are not limited to, pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, benzothiophenyl, furanyl, benzofuranyl, indolyl, azaindolyl (pyrrolopyridinyl), indazolyl, benzimidazolyl, pyrazolopyridinyl, triazolopyridinyl, benzotriazolyl, benzoxazolyl, benzothiazolyl, imidazopyridinyl, isoxazolopyridinylxanthinyl, guaninyl, quinolinyl, isoquinolinyl, tetrahydroquinolinyl, quinoxalinyl, and quinazolinyl. Heteroaryl groups include fused ring systems in which all of the rings are aromatic, for example, indolyl, and fused ring systems in which only one of the rings is aromatic, for example, 2,3-dihydroindolyl.

The term “halo” or “halogen” is intended to include F, Cl, Br, and I.

The term “heteroatom” is intended to include oxygen, nitrogen, sulfur, or phosphorus. In some embodiments, the heteroatom is selected from the group consisting of oxygen, nitrogen, and sulfur.

As used herein, the term “substituted” is intended to mean that one or more hydrogen atoms in the group indicated is replaced with one or more independently selected suitable substituents, provided that the normal valency of each atom to which the substituent(s) are attached is not exceeded, and that the substitution results in a stable compound. In some embodiments, optional substituents in the compounds described herein include but are not limited to halo, CN, NO₂, OH, NH₂, NHR₁₀₀, NR₁₀₀R₂₀₀, C₁₋₆haloalkyl, C₁₋₆haloalkoxy, C(O)NH₂, C(O)NHR₁₀₀, C(O)NR₁₀₀R₂₀₀, SO₂R₁₀₀, OR₁₀₀, SR₁₀₀, S(O)R₁₀₀, C(O)R₁₀₀, and C₁₋₆aliphatic; wherein R₁₀₀ and R₂₀₀ are each independently C₁₋₆aliphatic, for example C₁₋₆alkyl.

Where a protecting group (PG) is referred to, a person skilled in the art would readily understand what type of protecting group would be suitable.

The term “amine protecting group” as used herein is intended to mean a group that is capable of being readily removed to provide the NH₂ group of an amine group and protects the amine group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, acyl and acyloxy groups, for example acetyl, chloroacetyl, trichloroacetyl, o-nitrophenylacetyl, o-nitrophenoxy-acetyl, trifluoroacetyl, acetoacetyl, 4-chlorobutyryl, isobutyryl, picolinoyl, aminocaproyl, benzoyl, methoxy-carbonyl, 9-fluorenylmethoxycarbonyl, 2,2,2-trifluoroethoxycarbonyl, 2-trimethylsilylethoxy-carbonyl, tert-butyloxycarbonyl, benzyloxycarbonyl, p-nitrobenzyloxycarbonyl, 2,4-dichloro-benzyloxycarbonyl, and the like. Further examples include Cbz (carboxybenzyl), Nosyl (o- or p-nitrophenylsulfonyl), Bpoc (2-(4-biphenyl)isopropoxycarbonyl) and Dde (1-(4,4-dimethyl-2,6-dioxohexylidene)ethyl). In some embodiments, the amine protecting groups for the purposes described herein include (but are not limited to) tert-butyloxycarbonyl (t-Boc) and 9H-fluoren-9-ylmethoxycarbonyl (Fmoc).

The term “carboxyl protecting group” as used herein is intended to mean a group that is capable of being readily removed to provide the OH group of a carboxyl group and protects the carboxyl group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, alkyl and silyl groups, for example methyl, ethyl, tert-butyl, methoxymethyl, 2,2,2-trichloroethyl, benzyl, diphenylmethyl, trimethylsilyl, and tert-butyldimethylsilyl, and the like.

The term “carboxamide protecting group” as used herein is intended to mean a group that is capable of being readily removed to provide the NH₂ group of a carboxamide group and protects the carboxamide group against undesirable reaction during synthetic procedures. Such protecting groups are described in Protective Groups in Organic Synthesis edited by T. W. Greene et al. (John Wiley & Sons, 1999) and ‘Amino Acid-Protecting Groups’ by Fernando Albericio (with Albert Isidro-Llobet and Mercedes Alvarez) Chemical Reviews 2009 (109) 2455-2504. Examples include, but are not limited to, 9-xanthenyl (Xan), trityl (Trt), methyltrityl (Mtt), cyclopropyldimethylcarbinyl (Cpd), and dimethylcyclopropylmethyl (Dmcp).

The term “ester” refers to a carboxylic acid group where the hydrogen of the hydroxyl group has been replaced by a saturated, straight-chain (i.e. linear) or branched hydrocarbon group. Specific examples of alkyl groups are methyl, ethyl, propyl, iso-propyl, n-butyl, iso-butyl, sec-butyl, tert-butyl, n-pentyl, iso-pentyl, n-hexyl and 2,2-dimethylbutyl. The alkyl group may be a C₁-C₆ alkyl group. As used herein a wording defining the limits of a range of length such as, for example, “from 1 to 5” means any integer from 1 to 5, i.e. 1, 2, 3, 4 and 5. In other words, any range defined by two integers explicitly mentioned is meant to comprise and disclose any integer defining said limits and any integer comprised in said range. The alkyl group may be a branched alkyl group.

As used herein, ‘Ser’ refers to the amino acid serine and ‘Cys’ refers to the amino acid cysteine.

As used herein, ‘PEG’ refers to the polymer compound polyethylene glycol. Unless otherwise defined, reference to ‘PEG’ includes any length polymer of ethylene oxide. Reference to PEG also includes substituted PEG. In some embodiments, substituted PEG may be defined by formulas B-I or B-II as described herein.

As used herein, the term “and/or” means “and”, or “or”, or both.

The term “(s)” following a noun contemplates the singular and plural form, or both.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Administration and Dosage

In an embodiment of the invention, a therapeutically effective amount of any compound described herein is administered to the subject.

Administering refers to the physical introduction of a composition comprising a therapeutic agent to a subject, using any of the various methods and delivery systems known to those skilled in the art including those described herein. Pharmaceutical compositions may be formulated from compounds of the invention as described herein for any appropriate route of administration. Typically, in addition to the therapeutic agent (eg a compound described herein), a pharmaceutical composition comprises a pharmaceutically acceptable excipient, carrier and/or diluent. Examples of suitable components for inclusion in a pharmaceutical composition are described in Martindale—The Extra Pharmacopoeia (Pharmaceutical Press, London 1993) and Martin (ed.), Remington's Pharmaceutical Sciences.

Suitable routes of administration for implementing the defined methods include oral, intravenous, respiratory including inhalation and intranasal (e.g. in order to administer to the respiratory tract, particularly the lower respiratory tract, in particular the lung), intramuscular, topical, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Administering can also be performed, for example, once, a plurality of times, and/or over one or more extended periods.

Any composition described herein may be formulated for administration to the respiratory tract, in other words via a respiratory route. Where administration to all or part of the respiratory tract is contemplated, a skilled person will understand that this includes administration intranasally or via inhalation. The composition as described herein may be formulated for intranasal administration, including dry powder, sprays, mists, or aerosols.

Suitable formulations, wherein the carrier is a liquid, for administration, as for example, a nasal spray or as nasal drops, include aqueous or oily solutions of the active ingredient. Alternatively, the composition may be a dry powder and administered to the respiratory tract only as defined herein.

The selection of appropriate carriers depends upon the particular type of administration that is contemplated. For administration via the respiratory tract, e.g., the nasal mucosal surfaces, the compound can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or respiratory administration.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odour for the formulation. For nasal administration of solutions or suspensions according to the invention, various devices are available in the art for the generation of drops, droplets and sprays. For example, a compound or composition described herein can be administered into the nasal passages by means of a simple dropper (or pipet) that includes a glass, plastic or metal dispensing tube from which the contents are expelled drop by drop by means of air pressure provided by a manually powered pump, e.g., a flexible rubber bulb, attached to one end.

The tear secretions of the eye drain from the orbit into the nasal passages, thus, if desirable, a suitable pharmaceutically acceptable ophthalmic solution can be readily provided by the ordinary artisan as a carrier for the compound or composition described herein to be delivered and can be administered to the orbit of the eye in the form of eye drops to provide for both ophthalmic and intranasal administration.

In one embodiment, a premeasured unit dosage dispenser that includes a dropper or spray device containing a solution or suspension for delivery as drops or as a spray is prepared containing one or more doses of the drug to be administered. The invention also includes a kit containing one or more unit dehydrated doses of compound, together with any required salts and/or buffer agents, preservatives, colorants and the like, ready for preparation of a solution or suspension by the addition of a suitable amount of water. The water may be sterile or nonsterile, although sterile water is generally preferred.

The phrase ‘therapeutically effective amount’ or ‘effective amount’ generally refers to an amount of any compound described herein, a pharmaceutically acceptable salt, polymorph or prodrug thereof of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

For instance, for the treatment of tumours, a therapeutically effective amount of the compounds or compositions described herein can inhibit tumour growth by at least about 10%, by at least about 20%, by at least about 30%, by at least about 40%, by at least about 50%, by at least about 60%, by at least about 70%, by at least about 80%, or by at least about 90% or more, relative to untreated subjects. Alternatively, the treatments described herein may cause complete regression of the tumour mass. In other embodiments of the invention, tumour regression can be observed and continue for a period of at least about 10 days, at least about 20 days, at least about 30 days, at least about 40 days, at least about 50 days or at least about 60 days, at least about 70 days, at least about 80 days, at least about 90 days, at least about 100 days or longer.

A therapeutically effective amount of a drug may also include a “preventative” or “prophylactically effective amount,” which is any amount of any compound described herein administered to a subject at risk of developing a cancer (eg a subject having a pre-malignant condition) or of suffering a recurrence of cancer, that inhibits the development or recurrence of the cancer. In certain embodiments, the prophylactically effective amount prevents the development or recurrence of the cancer entirely. “Inhibiting” or “preventing” the development or recurrence of a cancer means either lessening the likelihood of the cancer's development or recurrence, or preventing the development or recurrence of the cancer entirely.

The exact amount of the therapeutically effective amount required will vary from subject to subject, depending on the species, age and general condition of the subject, mode of administration and the like. Thus, it may not be possible to specify an exact therapeutically effective amount. However, an appropriate therapeutically effective amount in any individual case may be determined by one of ordinary skill in the art using routine experimentation. In one aspect, the dose administered to a subject is any therapeutically effective amount that reduces symptoms associated with the cancer as a result of any one of a reduction in the number of cancer cells; a reduction in the tumour size; an inhibition (i.e., slow to some extent and preferably stop) of cancer cell infiltration into peripheral organs; an inhibition (i.e., slow to some extent and preferably stop) of tumour metastasis; an inhibition, to some extent, of tumour growth; or relieving, to some extent, of one or more of the symptoms associated with the cancer. Additionally or alternatively, the therapeutically effective amount may lead to increased survival of the subject.

In some embodiments, a therapeutically effective amount of any compound described herein for a human subject lies in the range of about 250 nmoles/kg body weight/dose to 0.005 nmoles/kg body weight/dose. Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, 5 nmoles, 2.5 nmoles, 0.5 nmoles, 0.25 nmoles, 0.1 nmoles or 0.05 nmoles/kg body weight/dose of the compound. Dosage regimes are adjusted to suit the exigencies of the situation and may be adjusted to produce the optimum therapeutic dose.

Typically, a therapeutically effective dosage is formulated to contain a concentration (by weight) of at least about 0.1% up to about 50% or more, and all combinations and sub-combinations of ranges therein. The compositions can be formulated to contain one or more compounds, or a pharmaceutically acceptable salt, polymorph or prodrug thereof in a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 16, 15, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue.

In some embodiments, treatment with any compound described herein is continued for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 18 months, at least about 24 months, at least about 3 years, at least about 5 years, or at least about 10 years.

It will be understood, that the specific dose level for any particular patient will depend upon a variety of factors including the activity of the specific compound employed, the age, body weight, general health, sex, diet, time of administration, route of administration, and rate of excretion, drug combination i.e. other drugs being used to treat the patient), and the severity of the particular disorder undergoing therapy.

The terms “treatment” or “treating” of a subject includes the application or administration of a compound of the invention to a subject with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

As used herein, minimising or preventing the progression of cancer means treating the subject so as to prevent or delay the recurrence or metastasis of a tumour, or to prevent growth of an existing tumour. Minimising or preventing the progression of cancer includes preventing or delaying the recurrence of cancer, or preventing growth of an existing tumour, following treatment of cancer. The recurrence that is being prevented includes a recurrence for example, in the tumour bed, following surgical excision. Alternatively, recurrence includes metastasis of the cancer in another part of the body. The terms “preventing recurrence” and “preventing relapse” as used herein, are interchangeable.

The present invention also includes methods of preventing the development of cancer in an individual. For example, the individual for whom prevention of cancer is required may be considered to be at risk of developing cancer, but does not yet have detectable cancer. An individual at risk of the development of cancer may be an individual with a family history of cancer, and/or an individual for whom genetic testing or other testing indicates a high risk or high likelihood of the development of cancer. The individual may have cancer stem cells but does not yet have any detectable tumours. It will be understood that methods of preventing the development of cancer include methods of delaying the onset of cancer in a subject.

The terms “subject”, “individual” and “patient” will be understood to be interchangeable. Although the invention finds application in humans, the invention is also useful for therapeutic veterinary purposes. The invention is useful for domestic or farm animals such as cattle, sheep, horses and poultry; for companion animals such as cats and dogs; and for zoo animals.

Cancer

The term “cancer” will be understood to include benign, pre-cancerous, pre-neoplastic or non-metastatic tumours or metastatic tumours.

In some embodiments, the type of cancer to be treated includes those having a benign, pre-cancerous, pre-neoplastic or non-metastatic tumour. A benign tumour will be understood to not be a malignant tumour and to not invade nearby tissue or spread to other parts of the body. Similarly non-metastatic cancer will be understood to not invade nearby tissue or spread to other parts of the body. “Pre-cancerous” or “pre-neoplasia” generally refers to a condition or a growth that typically precedes or develops into a cancer. A “pre-cancerous” growth may have cells that are characterized by abnormal cell cycle regulation, proliferation, or differentiation, which can be determined by markers of cell cycle.

In one embodiment, the cancer is a secondary cancer or metastases. The secondary cancer may be located in any organ or tissue, and particularly those organs or tissues having relatively higher hemodynamic pressures, such as lung, liver, kidney, pancreas, bowel and brain. The secondary cancer may be detected in the ascites fluid and/or lymph nodes.

In an embodiment of the invention, there is a step of identifying a subject having cancer. This may include the identification of a pre-cancerous or pre-neoplastic growth, secondary cancer or metastasis.

Pre-neoplastic, neoplastic and metastatic cancers are particular examples to which the methods of the invention may be applied. Broad examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours and Wilm's tumour.

Examples of particular cancers include but are not limited to adenocarcinoma, adenoma, adenofibroma, adenolymphoma, adontoma, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, ameloblastoma, angiokeratoma, angiolymphoid hyperplasia with eosinophilia, angioma sclerosing, angiomatosis, apudoma, anal cancer, angiosarcoma, aplastic anaemia, astrocytoma, ataxia-telangiectasia, basal cell carcinoma (skin), bladder cancer, bone cancers, bowel cancer, brain stem glioma, brain and CNS tumours, breast cancer, branchioma, CNS tumours, carcinoid tumours, cervical cancer, childhood brain tumours, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, carcinoma (e.g. Walker, basal cell, basosquamous, Brown-Pearce, ductal, Ehrlich tumour, Krebs 2, Merkel cell, mucinous, non-small cell lung, oat cell, papillary, scirrhous, bronchiolar, bronchogenic, squamous cell, and transitional cell), carcinosarcoma, cervical dysplasia, cystosarcoma phyllodies, cementoma, chordoma, choristoma, chondrosarcoma, chondroblastoma, craniopharyngioma, cholangioma, cholesteatoma, cylindroma, cystadenocarcinoma, cystadenoma, dermatofibrosarcoma-protuberans, desmoplastic-small-round-cell-tumour, ductal carcinoma, dysgerminoam, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anaemia, fibroma, fibrosarcoma, gall bladder cancer, gastric cancer, gastrointestinal cancers, gastrointestinal-carcinoid-tumour, genitourinary cancers, germ cell tumours, gestationaltrophoblastic-disease, glioma, gynaecological cancers, giant cell tumours, ganglioneuroma, glioma, glomangioma, granulosa cell tumour, gynandroblastoma, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, hamartoma, hemangioendothelioma, hemangioma, hemangiopericytoma, hemangiosarcoma, hemangiosarcoma, histiocytic disorders, histiocytosis malignant, histiocytoma, hepatoma, hidradenoma, hondrosarcoma, immunoproliferative small, opoma, ontraocular melanoma, islet cell cancer, Kaposi's sarcoma, kidney cancer, langerhan's cell-histiocytosis, laryngeal cancer, leiomyosarcoma, leukemia, li-fraumeni syndrome, lip cancer, liposarcoma, liver cancer, lung cancer, lymphedema, lymphoma, Hodgkin's lymphoma, non-Hodgkin's lymphoma, leigomyosarcoma, leukemia (e.g. B-cell, mixed cell, null-cell, T-cell, T-cell chronic, HTLV-Ilassociated, lymphangiosarcoma, lymphocytic acute, lymphocytic chronic, mast-cell and myeloid), leukosarcoma, leydig cell tumour, liposarcoma, leiomyoma, leiomyosarcoma, lymphangioma, lymphangiocytoma, lymphagioma, lymphagiomyoma, lymphangiosarcoma, male breast cancer, malignant-rhabdoid-tumour-of-kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, malignant carcinoid syndrome carcinoid heart disease, medulloblastoma, meningioma, melanoma, mesenchymoma, mesonephroma, mesothelioma, myoblastoma, myoma, myosarcoma, myxoma, myxosarcoma, nasal cancer, nasopharyngeal cancer, nephroblastoma, neuroblastoma, neurofibromatosis, Nijmegen breakage syndrome, non-melanoma skin cancer, non-small-cell-lung-cancer-(nsclc), neurilemmoma, neuroblastoma, neuroepithelioma, neurofibromatosis, neurofibroma, neuroma, neoplasms (e.g. bone, breast, digestive system, colorectal, liver), ocular cancers, oesophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal-tumours, pituitary cancer, polycythemia vera, prostate cancer, osteoma, osteosarcoma, ovarian carcinoma, papilloma, paraganglioma, paraganglioma nonchromaffin, pinealoma, plasmacytoma, protooncogene, rare-cancers-and-associated-disorders, renal cell carcinoma, retinoblastoma, rhabdomyosarcoma, Rothmund-Thomson syndrome, reticuloendotheliosis, rhabdomyoma, salivary gland cancer, sarcoma, schwannoma, Sezary syndrome, skin cancer, small cell lung cancer (sclc), small intestine cancer, soft tissue sarcoma, spinal cord tumours, squamous-cell-carcinoma-(skin), stomach cancer, synovial sarcoma, sarcoma (e.g. Ewing's experimental, Kaposi's and mast-cell sarcomas), Sertoli cell tumour, synovioma, testicular cancer, thymus cancer, thyroid cancer, transitional-cell-cancer-(bladder), transitional-cell-cancer-(renal-pelvis-/-ureter), trophoblastic cancer, teratoma, theca cell tumour, thymoma, trophoblastic tumour, urethral cancer, urinary system cancer, uroplakins, uterine sarcoma, uterus cancer, vaginal cancer, vulva cancer, Waldenstrom's-macroglobulinemia and Wilms' tumour.

A condition or symptom associated with the cancer may be any pathology that arises as a consequence of, preceding, or proceeding from the cancer. For example, where the cancer is a skin cancer, the condition or relevant symptom may be microbial infection. Where the cancer is a secondary tumour, the condition or symptom may relate to organ dysfunction of the relevant organ having tumour metastases. In one embodiment, the methods of treatment described herein are for the prevention of progression or treatment of a condition or symptom in an individual that is associated with a cancer in the individual.

The existence of, improvement in, treatment of, or minimisation of progression of cancer may be determined by any clinically or biochemically relevant method as described herein or known in the art. A positive response to treatment or a minimisation of progression of a cancer may be determined by any method known in the art and may include the determination of:
a reduction in the number of cancer cells;
a reduction in the tumour size;
an inhibition (i.e., slow to some extent and preferably stop) of cancer cell infiltration into peripheral organs;
an inhibition (i.e., slow to some extent and preferably stop) of tumour metastasis;
a reduction or complete prevention of tumour metastasis following removal of the primary tumour;
an inhibition, to some extent, of tumour growth;
relieving, to some extent, of one or more of the symptoms associated with the cancer; and/or
increased survival of the subject.

The determination of any of the above may be considered to be a positive response to any compound described herein.

In contrast, a negative response or a lack of response of a cancer to a treatment including any compound described herein may be determined by any method known in the art and may include the determination of:
no change or an increase in the number of cancer cells;
no change or an increase in the tumour size;
no change, continued or increased cancer cell infiltration into peripheral organs;
no change, continued or increased tumour metastasis;
no change or increased tumour metastasis, despite removal of the primary tumour;
no change or an increase of tumour growth;
no change or an increases of one or more of the symptoms associated with the cancer; and/or
no change or decreased survival of the subject.

The subject who has received the treatment for cancer may be in partial or complete remission. In other words, the subject, having received a treatment for cancer, as described above, may have a 10%, 20%, 30%, 40%, 50%, 60%, 70%, 80%, 90% or greater reduction in the measurable parameters of tumour growth as may be found on physical examination, radiologic study, or by biomarker levels from a blood or urine test. Alternatively, where the subject is in complete remission, there is a complete disappearance of all detectable manifestations of disease, such that the subject does not have any detectable signs of cancer. The subject may have substantially undetectable signs of cancer. A cancer that is “substantially undetectable” generally refers to a circumstance where therapy has depleted the size, volume or other physical measure of a cancer so that using relevant standard detection techniques such as in vivo imaging, the cancer, as a consequence of the therapy, is not clearly detectable.

The objective or outcome of treatment with any compound described herein may be to reduce the number of cancer cells; reduce the primary tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder.

Efficacy of treatment can be measured by assessing the duration of survival, time to disease progression, the response rates (RR), duration of response, and/or quality of life.

In one embodiment, the method is particularly useful for delaying cancer progression. In one embodiment, the method is particularly useful for extending survival of the subject, including overall survival as well as progression free survival. It will be understood that overall survival is the length of time from either the date of diagnosis or the start of treatment of a cancer, that patients diagnosed with the cancer are still alive. It will be understood that progression free survival is the length of time during and after the treatment of a cancer that a patient lives with the disease but it does not get worse.

Survival analysis can be performed using well known techniques in the art including the Kaplan-Meier method. The Kaplan-Meier method estimates the survival function from life-time data. In medical research, it can be used to measure the fraction of patients living for a certain amount of time after treatment. A plot of the Kaplan-Meier method of the survival function is a series of horizontal steps of declining magnitude which, when a large enough sample is taken, approaches the true survival function for that population. The value of the survival function between successive distinct sampled observations (“clicks”) is assumed to be constant.

An important advantage of the Kaplan-Meier curve is that the method can take into account “censored” data-losses from the sample before the final outcome is observed (for instance, if a patient withdraws from a study). On the plot, small vertical tick-marks indicate losses, where patient data has been censored. When no truncation or censoring occurs, the Kaplan-Meier curve is equivalent to the empirical distribution.

In one embodiment, the method is particularly useful for providing a complete response to therapy whereby all signs of cancer in response to treatment have disappeared. This does not always mean the cancer has been cured. In one embodiment, the method is particularly useful for providing a partial response to therapy whereby there has been a decrease in the size of one or more tumours or lesions, or in the extent of cancer in the body, in response to treatment.

Kits

In another embodiment there is provided a kit or article of manufacture comprising any compound described herein, a pharmaceutically acceptable salt, diluent or excipient and/or pharmaceutical composition as described above. Further, the kit may comprise instructions for use in any method or use of the invention as described herein.

In other embodiments there is provided a kit for use in a therapeutic and/or prophylactic application mentioned above, the kit comprising:
a container holding a therapeutic composition in the form of any compound described herein, or a pharmaceutically acceptable salt, diluent or excipient or pharmaceutical composition;
a label or package insert with instructions for use.

In certain embodiments the kit may contain one or more further active principles or ingredients for treatment of cancer.

In an embodiment, the therapeutic composition of the kit is formulated for administration to the respiratory tract, preferably by inhalation.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic or prophylactic composition can be used to treat a cancer described herein.

The kit may comprise (a) a therapeutic or prophylactic composition; and (b) a second container with a second active principle or ingredient contained therein. The kit in this embodiment of the invention may further comprise a package insert indicating the composition and other active principle can be used to treat a cancer or prevent progression of a cancer described herein.

As used herein, the following compounds are described in the table below and specific structures shown elsewhere herein and contemplated in any method or use of the invention.

| | Com- |
|---|---|
| | pound |
| Compound Structure | ID |
| 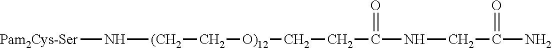 | A101/ com- pound 1 |
| 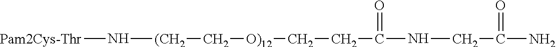 | A102 |
| 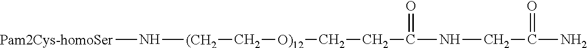 | A103 |
| 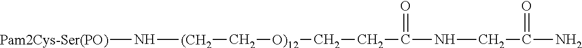 | A104 |
| 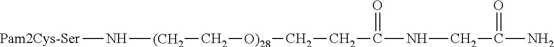 | A105 |
| 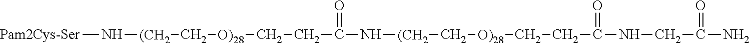 | A106 |
| 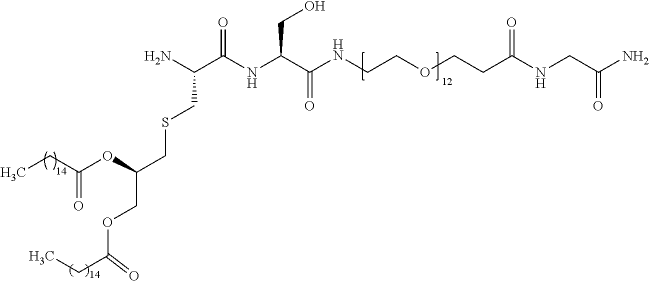 | A107 |
| 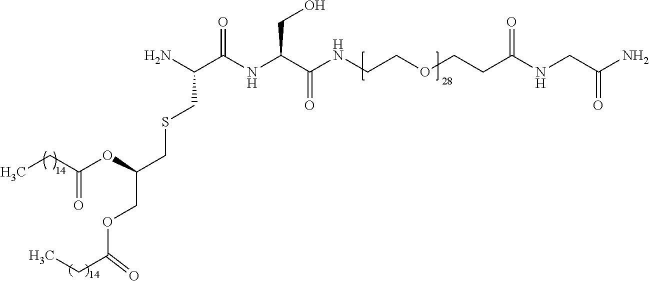 | A108 |
| 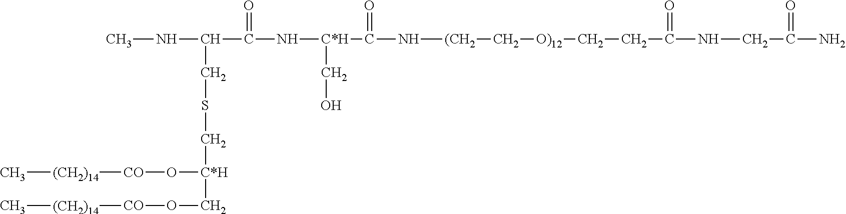 | A109 |
| 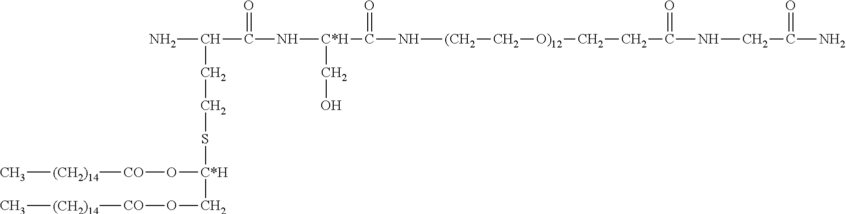 | A110 |
| 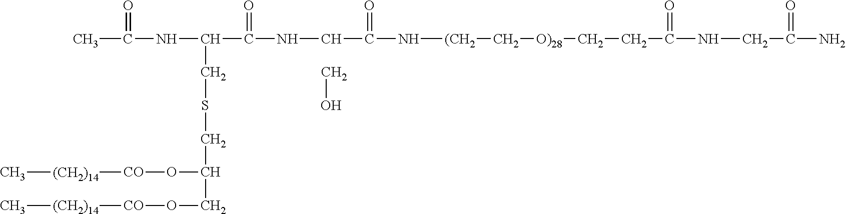 | A111 |
| 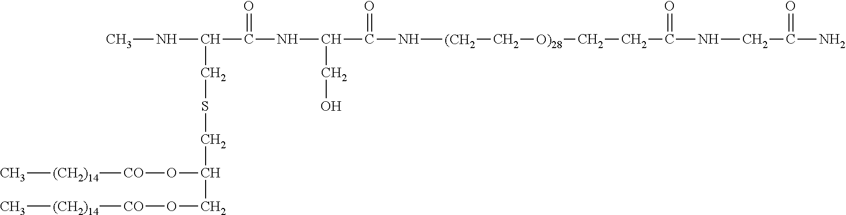 | A112 |
| 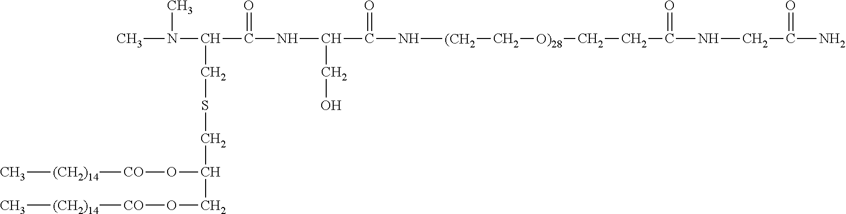 | A113 |
| 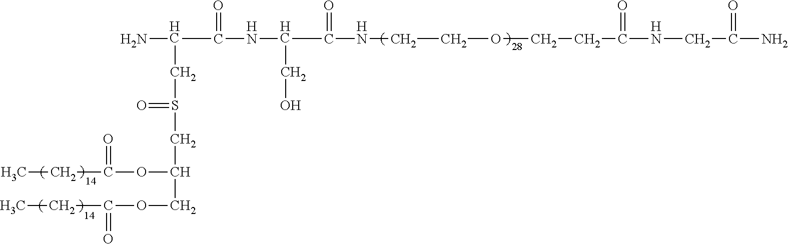 [SMILES: CCC(=O)OCC(CS(=O)CC(N)C(=O)NC(CO)C(=O)NCCOCCC(=O)NCC(N)=O)OC(=O)CC] | A114 |
| 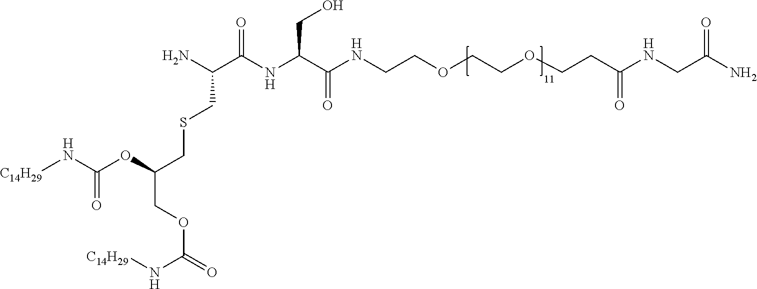 | A115 |
| 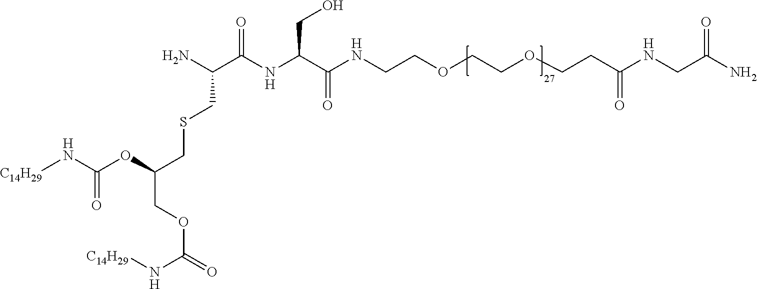 | A116 |
| 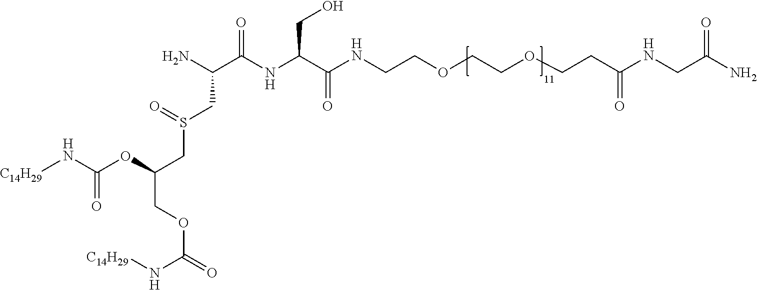 | A117 |
| 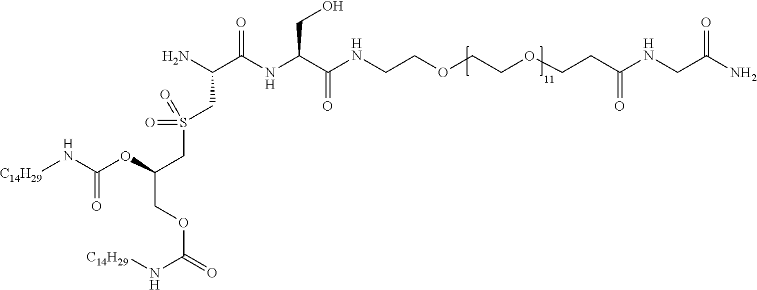 | A118 |
| 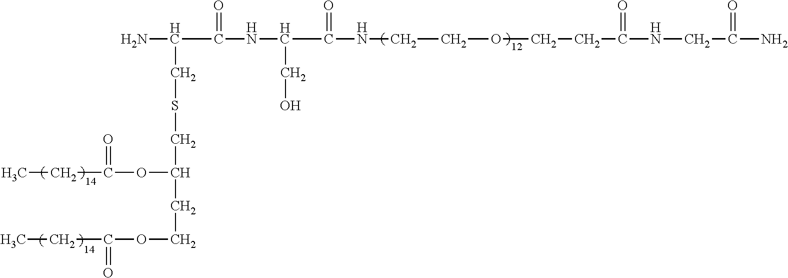 [SMILES: CCC(=O)OCCC(CSCC(N)C(=O)NC(CO)C(=O)NCCOCCC(=O)NCC(N)=O)OC(=O)CC] | A201 |
| 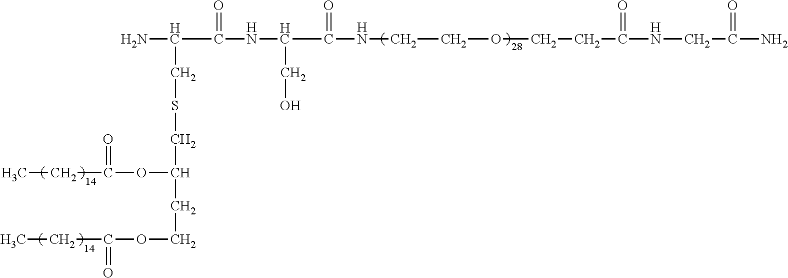 [SMILES: CCC(=O)OCCC(CSCC(N)C(=O)NC(CO)C(=O)NCCOCCC(=O)NCC(N)=O)OC(=O)CC] | A202 |
| 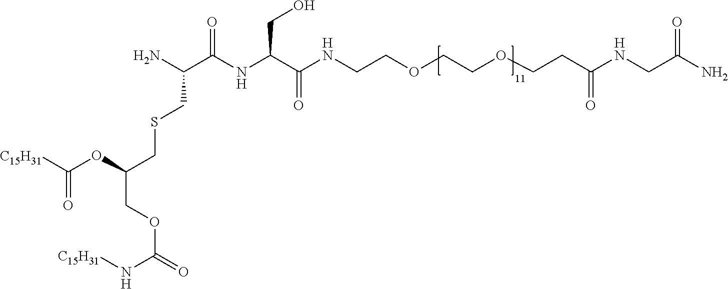 | A203 |
| 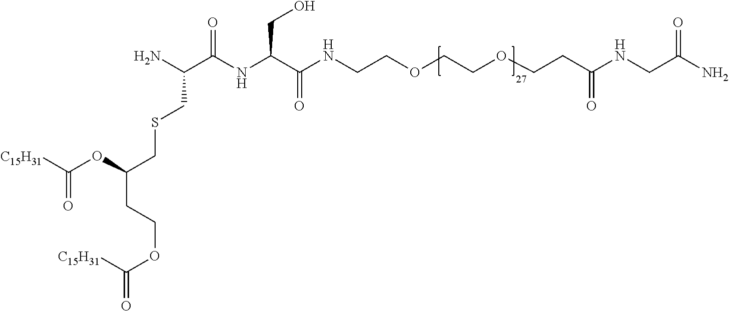 | A204 |
| 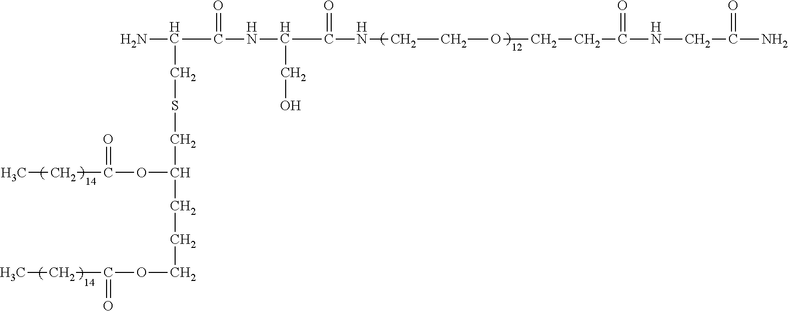 [SMILES: CCC(=O)OCCCC(CSCC(N)C(=O)NC(CO)C(=O)NCCOCCC(=O)NCC(N)=O)OC(=O)CC] | A205 |
| 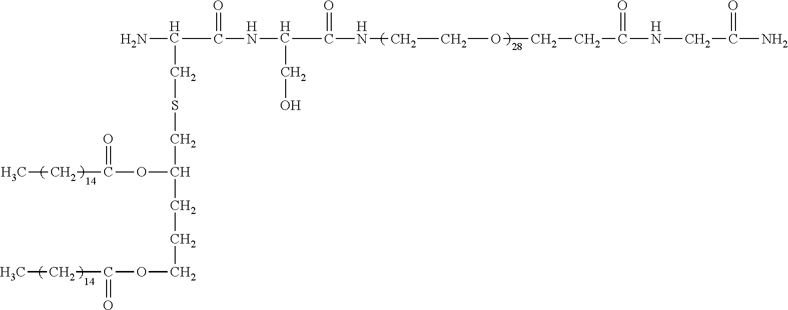 [SMILES: CCC(=O)OCCCC(CSCC(N)C(=O)NC(CO)C(=O)NCCOCCC(=O)NCC(N)=O)OC(=O)CC] | A206 |
| 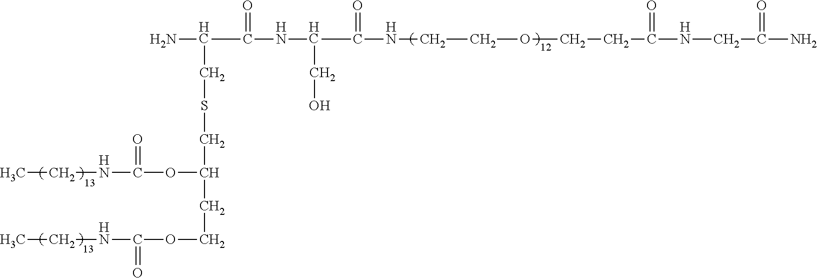 [SMILES: CCNC(=O)OCCC(CSCC(N)C(=O)NC(CO)C(=O)NCCOCCC(=O)NCC(N)=O)OC(=O)NCC] | A207 |
| 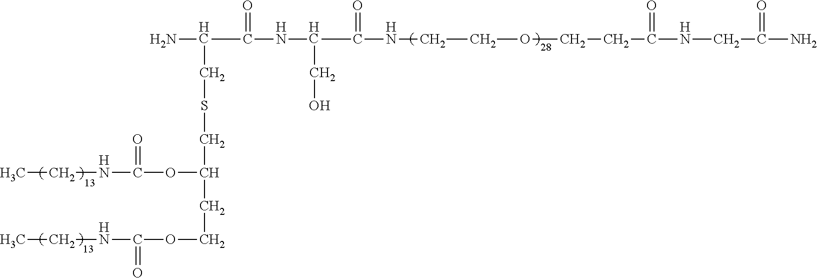 [SMILES: CCNC(=O)OCCC(CSCC(N)C(=O)NC(CO)C(=O)NCCOCCC(=O)NCC(N)=O)OC(=O)NCC] | A208 |
| 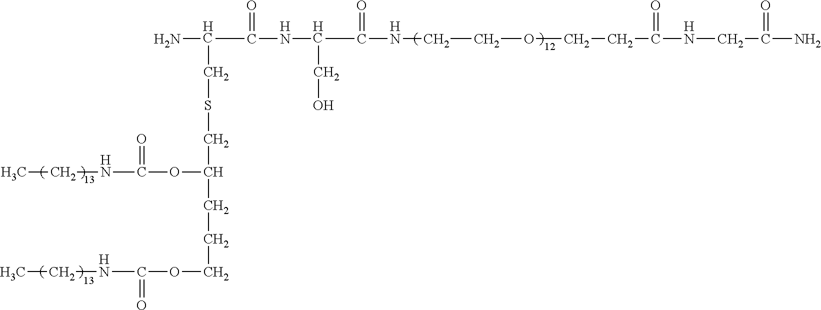 [SMILES: CCNC(=O)OCCCC(CSCC(N)C(=O)NC(CO)C(=O)NCCOCCC(=O)NCC(N)=O)OC(=O)NCC] | A209 |
| 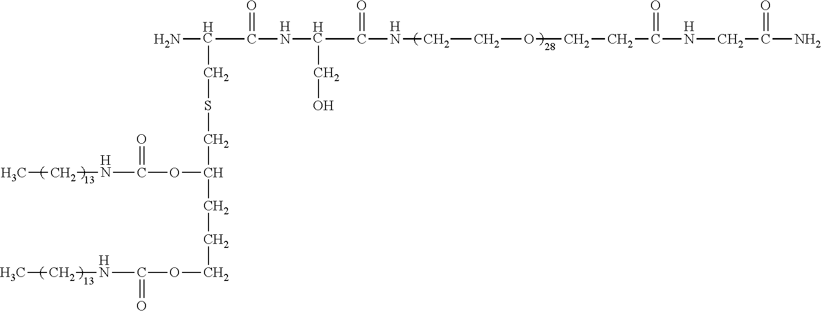 [SMILES: CCNC(=O)OCCCC(CSCC(N)C(=O)NC(CO)C(=O)NCCOCCC(=O)NCC(N)=O)OC(=O)NCC] | A210 |
| 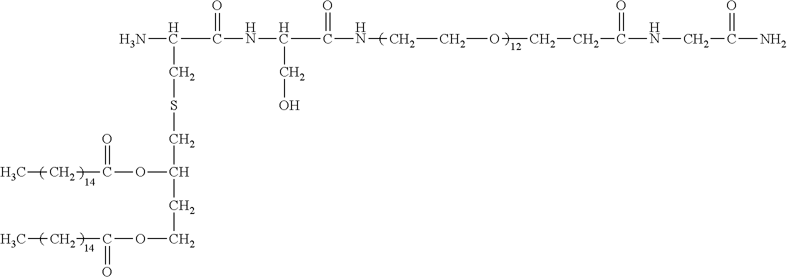 [SMILES: CCC(=O)OCCC(CSCC(N)C(=O)NC(CO)C(=O)NCCOCCC(=O)NCC(N)=O)OC(=O)CC] | A211 |
| 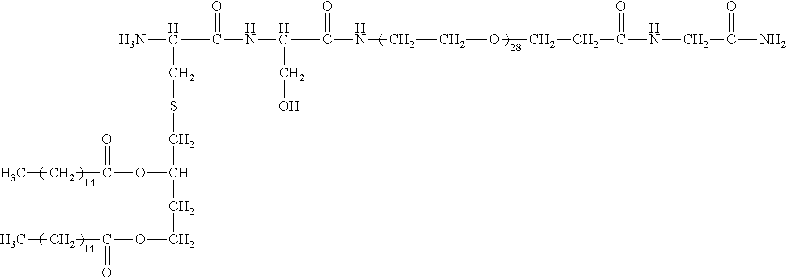 [SMILES: CCC(=O)OCCC(CSCC(N)C(=O)NC(CO)C(=O)NCCOCCC(=O)NCC(N)=O)OC(=O)CC] | A212 |
| 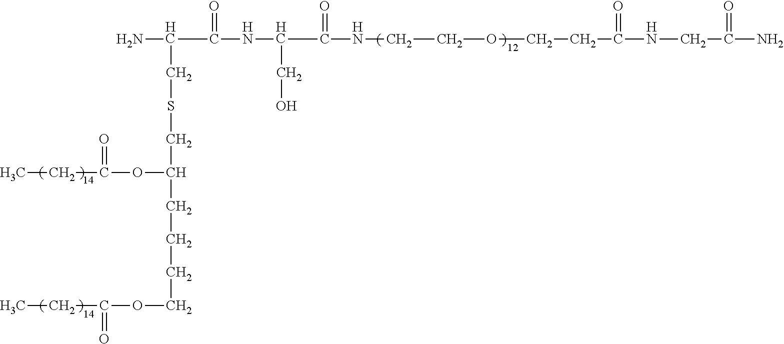 [SMILES: CCC(=O)OCCCCC(CSCC(N)C(=O)NC(CO)C(=O)NCCOCCC(=O)NCC(N)=O)OC(=O)CC] | A213 |
| 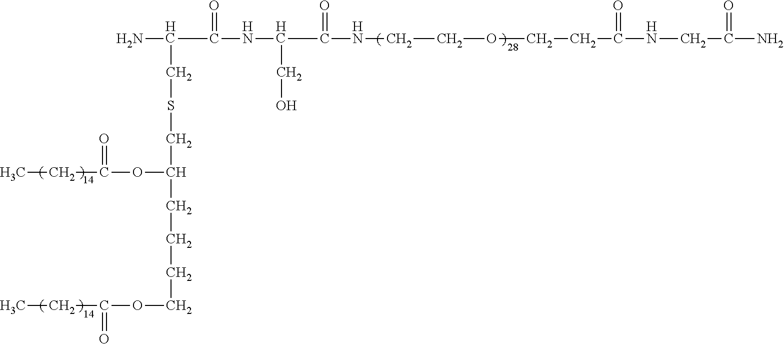 [SMILES: CCC(=O)OCCCCC(CSCC(N)C(=O)NC(CO)C(=O)NCCOCCC(=O)NCC(N)=O)OC(=O)CC] | A214 |
| 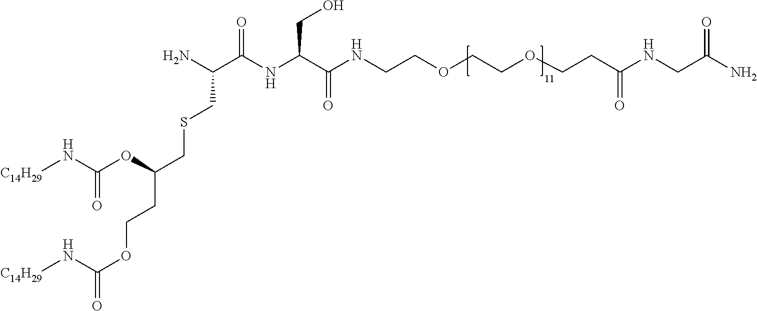 | A215 |
| 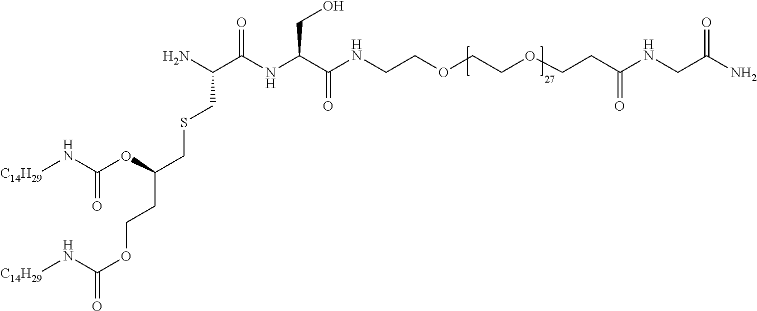 | A216 |
| 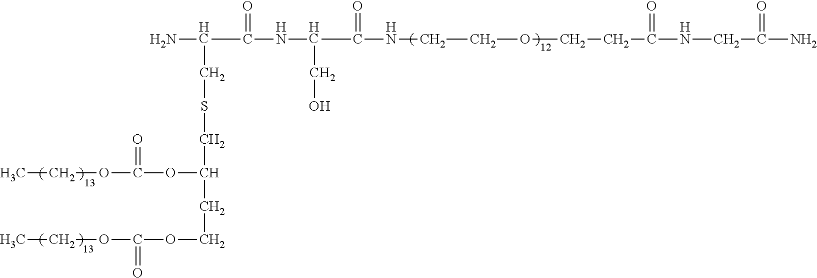 [SMILES: CCOC(=O)OCCC(CSCC(N)C(=O)NC(CO)C(=O)NCCOCCC(=O)NCC(N)=O)OC(=O)OCC] | A217 |
| 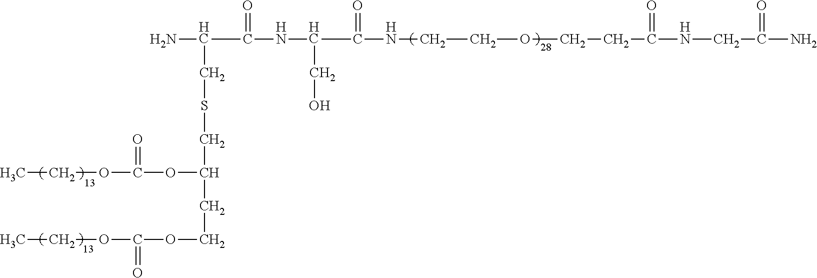 [SMILES: CCOC(=O)OCCC(CSCC(N)C(=O)NC(CO)C(=O)NCCOCCC(=O)NCC(N)=O)OC(=O)OCC] | A218 |
| 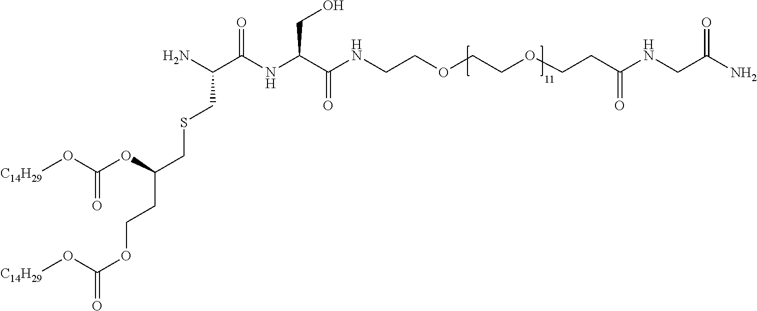 | A219 |
| 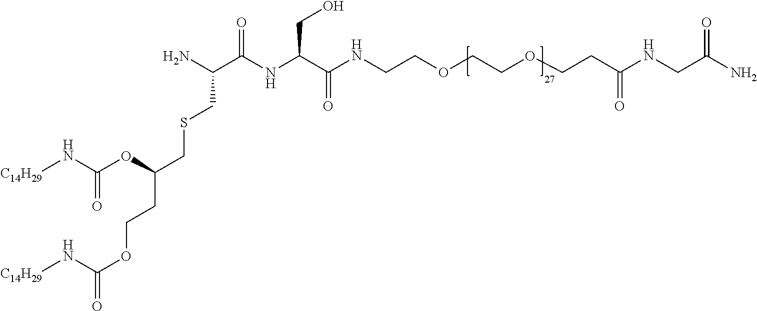 | A220 |
| 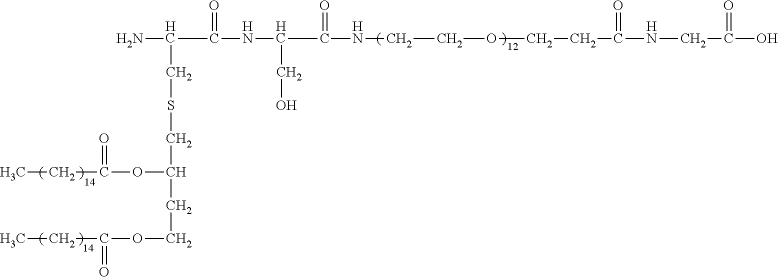 [SMILES: CCC(=O)OCCC(CSCC(N)C(=O)NC(CO)C(=O)NCCOCCC(=O)NCC(=O)O)OC(=O)CC] | A221 |
| 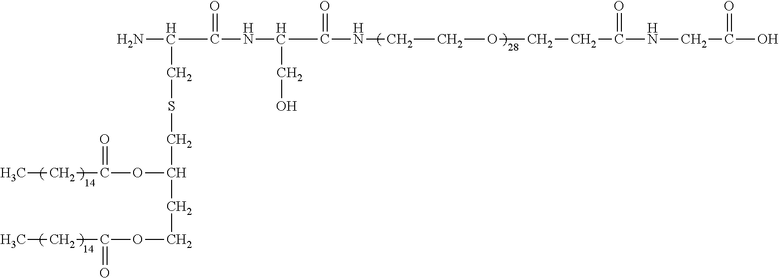 [SMILES: CCC(=O)OCCC(CSCC(N)C(=O)NC(CO)C(=O)NCCOCCC(=O)NCC(=O)O)OC(=O)CC] | A222 |
| 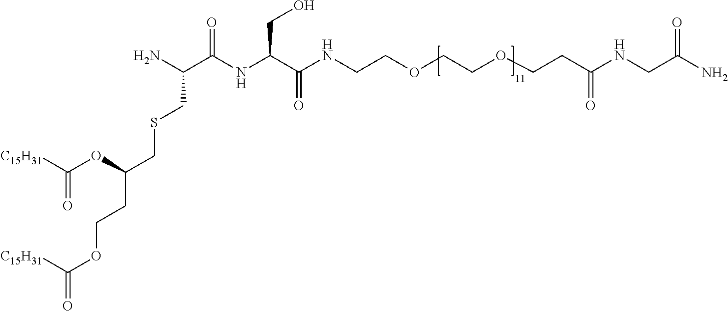 | A223 |
| 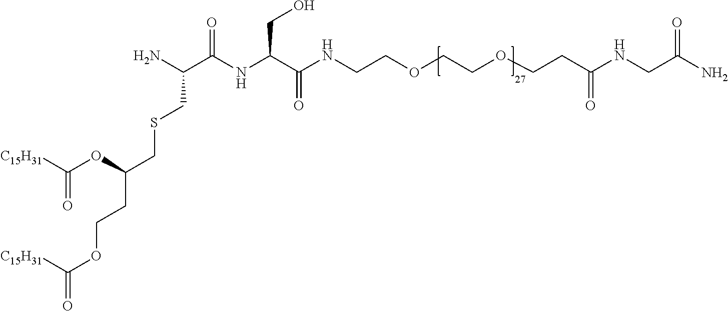 | A224 |
| 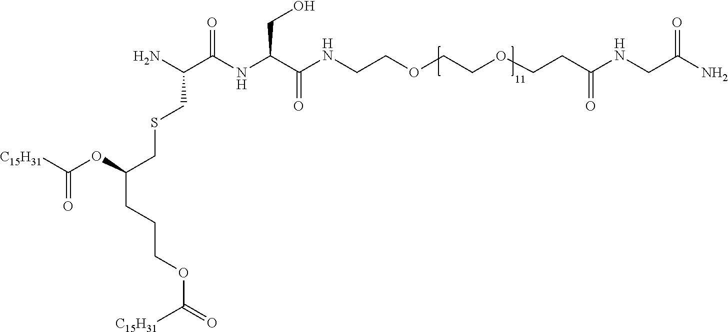 | A225 |
| 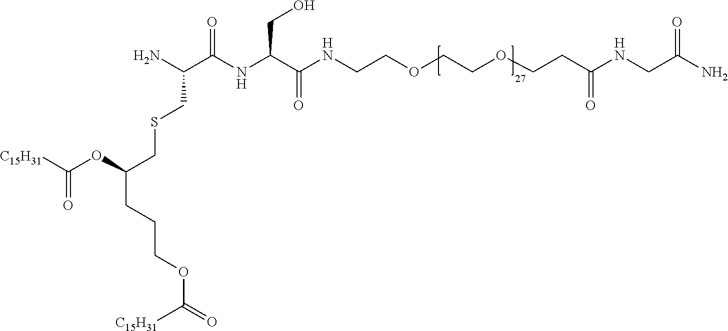 | A226 |
| 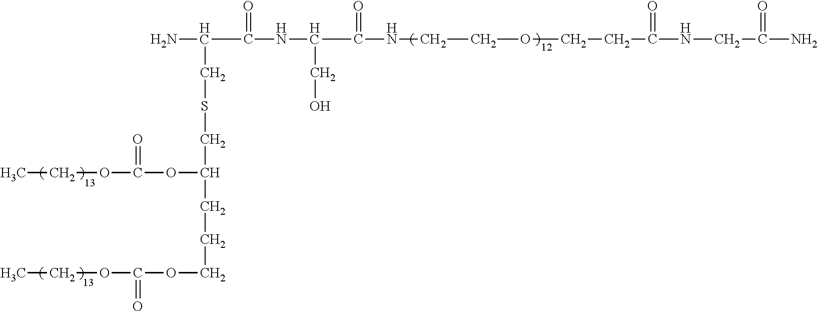 [SMILES: CCOC(=O)OCCCC(CSCC(N)C(=O)NC(CO)C(=O)NCCOCCC(=O)NCC(N)=O)OC(=O)OCC] | A227 |
| 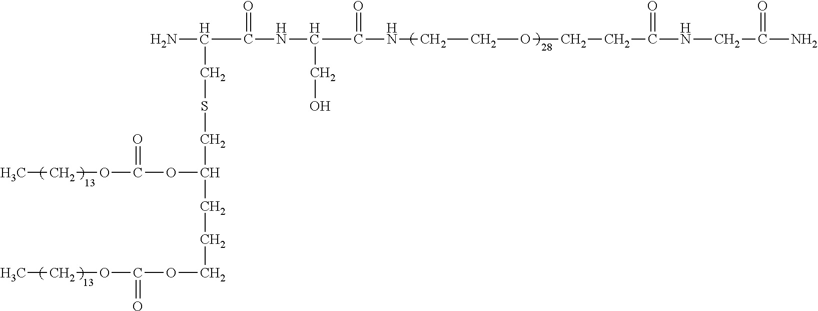 [SMILES: CCOC(=O)OCCCC(CSCC(N)C(=O)NC(CO)C(=O)NCCOCCC(=O)NCC(N)=O)OC(=O)OCC] | A228 |
| 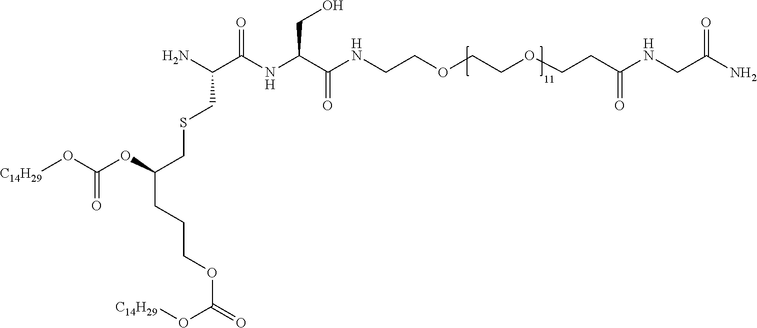 | A229 |
| 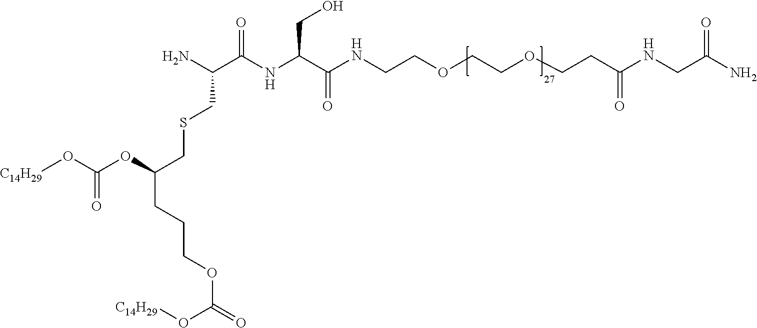 | A230 |
| 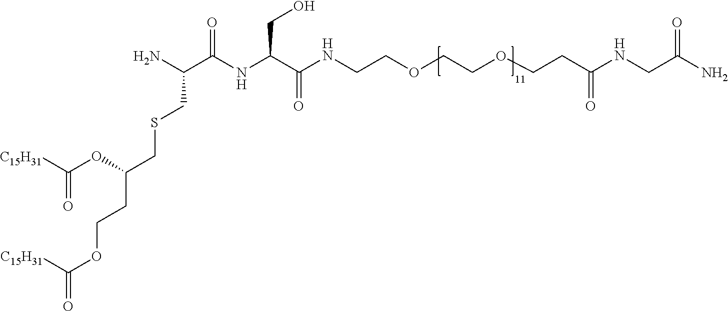 | A231 |
| 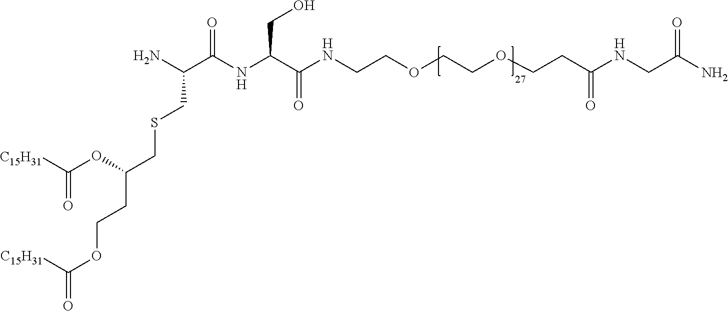 | A232 |

Compounds of the invention may be prepared by techniques known in the art. For example, compounds of the invention including any one of formulas (I)-(XIX) comprising an A1 moiety may be prepared by techniques described in WO2019/119067, the entire contents of which are hereby incorporated by reference.

Compounds of the invention including any one of formulas (I)-(XIX) comprising an A2 moiety may be provided by coupling a compound of the formula A2-I:

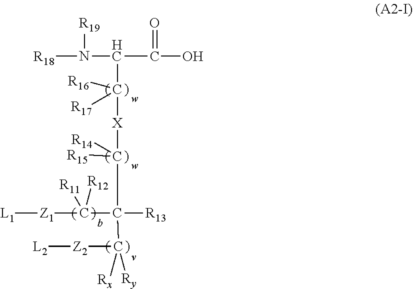

wherein L₁, L₂, Z₁, Z₂, v, b, w, z, Rₓ, R_{y}, R₁₁, R₁₂, R₁₃, R₁₄, R₁₅, R₁₆, R₁₇, R₁₈ and X have the meanings as defined for any compound of the invention defined herein and R₁₉ is an amino protecting group
with a compound of formula (YB-1):

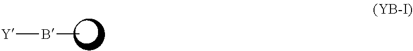

wherein
Y′ is

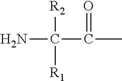

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
B′ is a Polyethylene Glycol (PEG); and
[symbol] is a solid support resin.

In some embodiments, B′ comprises a substituted PEG of Formula B-I. In these embodiments, the following sequence of solid phase reactions may be employed:
a) Optionally coupling 1 to 10 alpha amino acids or compounds derived from a natural alpha amino acid, that constitutes L, to a solid phase resin using Fmoc chemistry
b) Coupling PG-NH—(CH₂)ₚ—O—(CH₂CH₂O)ₙ—(CH₂)ₘ—COOH to a solid phase resin or substituted resin if L is present, wherein PG represents an amino protecting group compatible with Fmoc chemistry;
c) Removing PG;
d) Coupling PG-NH—CR₁₃R₁₄—COOH, wherein PG′ represents an amino protecting group compatible with Fmoc chemistry;
e) Removing PG′;
f) Coupling an acid of the formula (A-1);
g) Optionally removing R₁₉ and optionally acylating and/or alkylating to introduce R₁₈ and/or R₁₉; and
h) Removing the compound from the solid phase support

In some embodiments, B′ comprises a substituted PEG according to formula (B-II) and the following sequence of solid phase reactions may be employed:
a) Optionally coupling 1 to 10 alpha amino acids or compounds derived from a natural alpha amino acid, that constitute L, to a solid phase resin using Fmoc chemistry
b) Coupling PG-NH—(CH₂)ₜ—O—(CH₂CH₂O)ₖ—(CH₂)ₕ—COOH to a solid phase resin or substituted resin if L is present, wherein PG represents an amino protecting group compatible with Fmoc chemistry;
c) Removing PG;
d) Coupling PG′-NH—(CH₂)ₚ—O—(CH₂CH₂O)ₙ—(CH₂)ₘ—COOH, wherein PG′ represents an amino protecting group compatible with Fmoc chemistry;
e) Removing PG′;
f) Coupling PG″-NH—CR₁₃R₁₄—COOH, wherein PG″ represents an amino protecting group compatible with Fmoc chemistry;
g) Removing PG″;
h) Coupling an acid of the formula (A-1);
i) Optionally removing R₁₉ and optionally acylating and/or alkylating to incorporate R₁₈ and/or R₁₉; and
j) Removing the compound from the solid phase resin.

It will be appreciated that the exact sequence of events can be varied from that outlined, and additional steps added where necessary and synthetically expedient, for example oxidation of the cysteine sulfur to the sulfoxide or sulfone.

In some embodiments, the compound of formula A2-1 is provided in the form of a compound of formula A2-II:

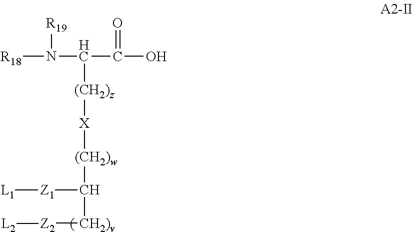

wherein L₁, L₂, X, v, w and R₁₈ are as defined for the compound of formula A-1 above, Z₁ and Z₂ are independently selected from —NHC(O)—, —C(O)NH—, —OC(O)—, —C(O)O—, —NHC(O)O— and —OC(O)O—.

The compound of formula A2-II may be prepared by the synthesis shown in Scheme 1.

Scheme 1 describes the synthesis of embodiments of the compound of formula A2-II, wherein
X is S,
L₁-Z₁ are —OC(O)E-(CH₂)_{g}—CH₃, wherein E is —O— or —NH— and g is 10, 11, 12, 13, 14, 15, 16, 17 or 18; L₂-Z₂ are —OC(O)E-(CH₂)_{g}—CH₃, wherein E is —O— or —NH— and g is 10, 11, 12, 13, 14, 15, 16, 17 or 18; and
R₁₉ is PG3, which is an amino protecting group.

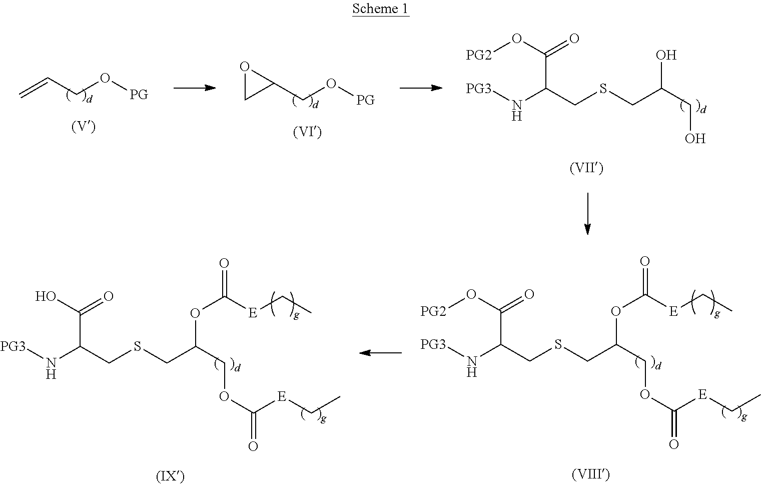

Reaction of protected alkene alcohols of the formula (V′), where PG is a suitable protecting group, for example a silyl group such as TBDMS, forms an epoxide of the formula (VI′). It will be appreciated that the epoxide formation maybe carried out to give the product racemically or to give enantioenriched material. If a racemic or scalemic mixture of enantiomers is produced preparative chiral chromatography is employed to separate the enantiomers if required.

Epoxides of the formula (VI′) are reacted with suitably protected cystine analogues, for example tert-butyl N-(((9H-fluoren-9-yl)methoxy)carbonyl)-S—(((R)-2-((((9H-fluoren-9-yl)methoxy)carbonyl)amino)-3-(tert-butoxy)-3-oxopropyl)thio)-D-cysteinate, where PG2 is a tert-butyl ester and PG3 is Fmoc, under reducing conditions to give alcohols of the formula (VII′). It will be appreciated that alcohols of the formula (VII′) can be comprised of more than one stereoisomer and where stereoisomers are present these can be separated by chiral preparative chromatography as required.

Alcohols of the formula (VII′) can be acylated to give carbonyl containing adducts of the formula (VIII′) using suitable reagents. Where esters are required, acid chlorides can be reacted in the presence of suitable bases and solvents; where carbamates are required isocyanates can be reacted in the presence of suitable bases and solvents and where carbonates are required chloroformates can be reacted in the presence of suitable bases and solvents. Carbonyl containing adducts of the formula (VIII′) can then be deprotected to reveal carboxylic acids of the formula (IX′) using suitable reagents, for example where PG2 is tert-butyl, trifluoroacetic acid can be used to preferentially remove the tert-butyl group.

Acids of the formula (IX′) can then be used as reagents in solid phase synthesis to add groups of formula Y and B.

Compounds of the invention including any one of formulas (I)-(XIX) comprising an A2 moiety wherein z is 1, w is 1 and b is 0, may be provided by preparing a resin bound peptide of the following formula:

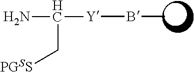

wherein
Y′ is

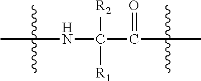

wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
B′ is a Polyethylene Glycol (PEG);
PG^{s} is H or a sulphur protecting group, such as tert-butyl; and
[symbol] is a solid support resin.

Following optional sulphur deprotection, this resin bound peptide may be reacted with a 1,2-epoxy-alkanol of the following formula:

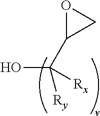

wherein Rₓ, R_{y} and v have the meanings given for moiety A2, for example as defined in formula (I)
to provide an alkylated thiol of formula S-1:

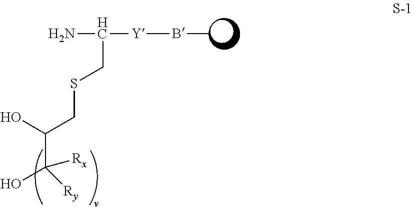

wherein Y′ and B′ have the meaning given above, and v has the meaning given for moiety A2, or a sulfone or sulfoxide thereof.

The diol moieties of resin bound compound S-1 may be further reacted to provide a compound of the invention, for example, by diol functionalisation with palmitic groups or lauryl carbamate groups, etc.

### EXAMPLES

The following examples 1-6 describe various properties of the compounds of the invention. The compounds tested in these examples were prepared as described in WO2019/119067 or in synthesis Example 7.

### Example 1—Effect of Synthetic Compound A101/Compound 1 on Tumour Growth, Survival and Metastasis

Here the inventors set out to investigate the anti-tumour effect of a synthetic compound referred to as compound A101 (referred to in the Examples and Figures as Compound 1).

Material and Methods

Mice

C57BL/6 or Balb/c Wild-type (WT) mice were purchased from Walter and Eliza Hall Institute for Medical Research or bred in house and maintained at the QIMR Berghofer Medical Research Institute. Mice greater than 8 weeks of age were sex-matched to the appropriate models. The number of mice in each group treatment or strain of mice for each experiment is indicated in the figure legends. In all studies, no mice were excluded based on pre-established criteria and randomization was applied immediately prior to treatment in therapy experiments. Experiments were conducted as approved by the QIMR Berghofer Medical Research Institute Animal Ethics Committee.

Cell Culture

Mouse B16F10 (melanoma), MC38 (colon adenocarcinoma) and 4T1.2 (breast cancer) cells were grown in Dulbecco's Modified Eagle Medium (DMEM) supplemented with 10% Fetal Calf Serum (Bovogen), 1% Glutamine (Gibco), and 1% Penicillin/Streptomycin (Gibco). B16F10 and 4T1.2 cells were maintained at 37° C., 5% CO2. MC38 cells were maintained at 37° C., 10% CO2. All cell lines were routinely tested negative for Mycoplasma, but cell line authentication was not routinely performed.

Subcutaneous Tumour Models

For primary tumour growth experiments MC38 (1×10⁶) or B16F10 (1×10⁵) cells were subcutaneous (s.c.) injected into mice in a final volume of 100 μl (day 0). Treatment of mice commenced as indicated in figures or legends. Digital calipers were used to measure the perpendicular diameters of each individual tumour. The tumour size was calculated and is presented as mean±SEM. When tumours reached a size of 150 mm² mice were sacrificed.

4T1.2 Breast Cancer Metastasis Model

5×10⁴ 4T1.2 cells were injected into the 4th mammary fat pad in a volume of 50 μl (day 0). On day 12 after injection, primary tumours were surgically removed under isoflurane anaesthesia. Mice were injected treated as indicated in figure legends on day 15, 18 and 21 after tumour cell injection. All mice were sacrificed on day 26 after tumour cell injection for evaluation of metastatic burden. For this, macroscopic visible lung metastases were counted.

Intratumoural Injection of Compound 1

For this 100 μl compound 1 solution in saline was injected into the tumour. Doses and treatment schedules are depicted in figures and/or legends.

Intravenous Injection of Compound 1

For this 10 μg compound 1 in 200 μl saline were injected into the lateral tail vein. Treatment schedules are depicted in figures and/or legends.

Results

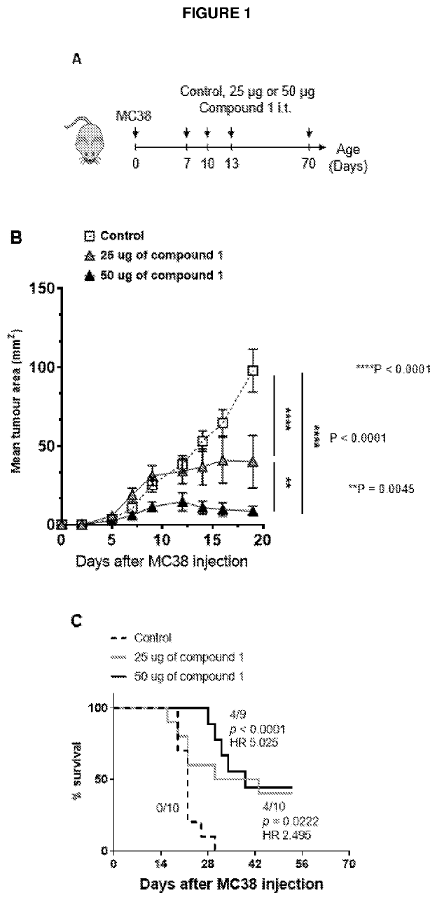

C57BL/6 WT mice were s.c injected with highly immunogenic MC38 colon carcinoma cells. Once tumours were palpable (˜5 mm in diameter), mice were randomized into three groups receiving three intratumoral injections of vehicle, 25 μg or 50 μg compound 1 in 100 μl saline (FIG. 1A). A dose dependent inhibition of MC38 tumour growth was observed along with significantly improved overall survival of mice treated with compound 1 compared to vehicle control (FIG. 1B, C).

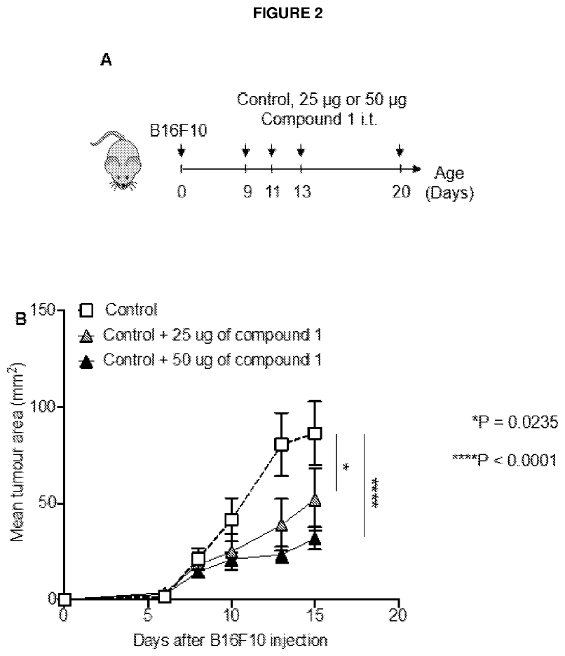

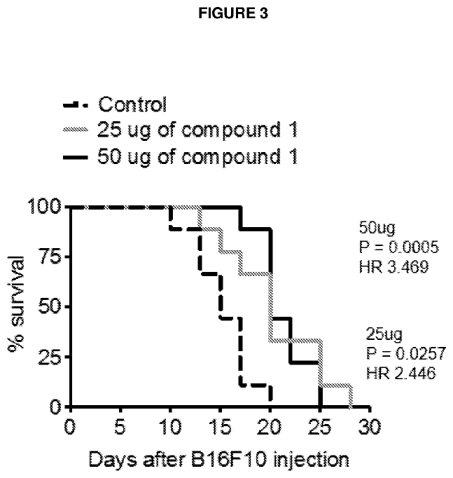

In the next experiment, the poorly immunogenic B16F10 melanoma cell line was s.c injected into C57BL/6 WT mice. When tumours were palpable, mice were randomized into three groups and treated as indicated (FIG. 2A). Because B16F10 melanoma are extremely difficult to treat, four doses of Compound 1 were injected. A dose-dependent reduction in tumour growth was observed (FIG. 2B). Although, compound 1 treatment did not cause complete tumour rejection, importantly it significantly improved the survival of mice (FIG. 3).

Taken together, these data indicate that compound 1 has single agent anti-tumour efficacy, when locally injected into the tumour microenvironment, in both highly and poorly immunogenic pre-clinical models of cancer.

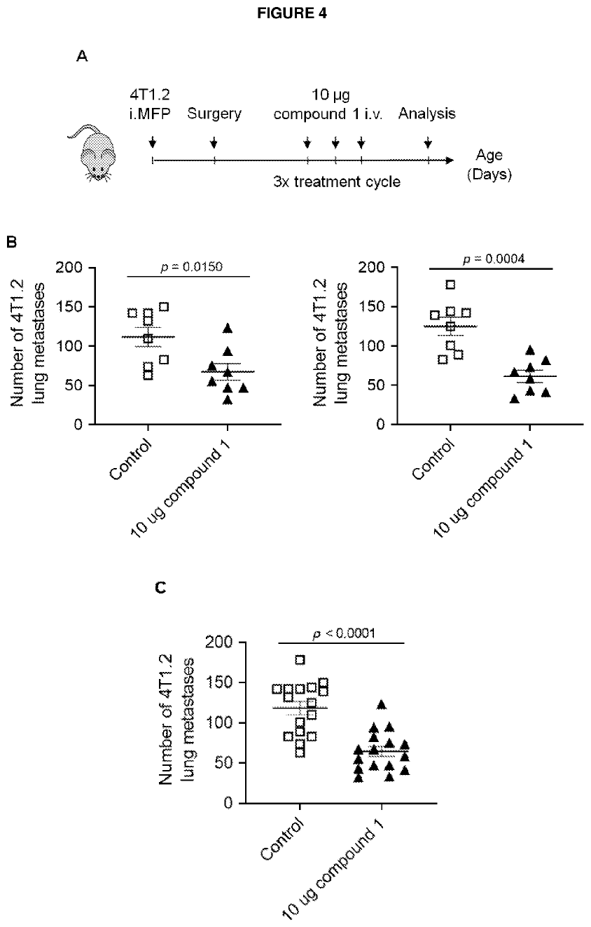

So far, the data demonstrates that compound 1 has single agent efficacy when tested in models of MC38 and B16F10 driven cancers. Because metastatic dissemination of tumour cells is the leading cause of death in cancer patients, studies were conducted to evaluate the anti-metastatic potential of compound 1. For this Balb/c WT mice were injected into the 4th mammary fat pad with the highly aggressive 4T1.2 breast cancer cell line. As per the typical treatment approach in patients, the primary tumours were removed and mice were subsequently treated with 10 μg of compound 1 in 200 μl saline i.v. as indicated (FIG. 4A). All mice were sacrificed 14 days after surgery and the number of lung metastases was evaluated. Systemic treatment with compound 1 significantly reduced the number of 4T1.2 lung metastasis (FIG. 4B-C) without significantly affecting weight loss (data not shown).

In summary, the data shows that compound 1 has strong anti-tumour efficacy upon local (intertumoral) and systemic (intravenous) administration not only against primary tumours but also in a model of metastasis.

### Example 2. Compound A108 has Anti-Tumour Activity in the MC38 Model

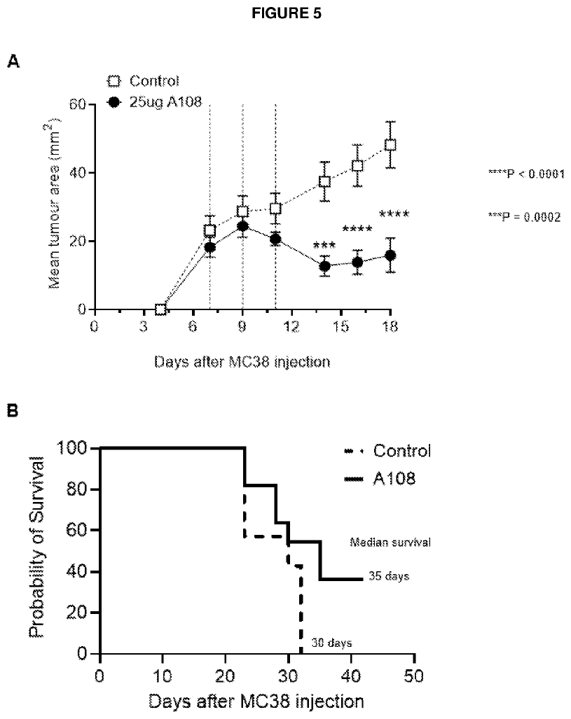

C57BL/6 WT mice (n=10-14) were injected subcutaneously with highly immunogenic MC38 colon carcinoma cells. Once tumours were palpable (˜3-5 mm in diameter), mice were randomized into tour groups receiving three intra-tumoral injections of vehicle or 25 μg compound A108 in 100 μl saline. These studies show that compound A108 inhibits MC38 tumour growth (FIG. 5A). Compound A108 also shows improvement to the overall survival of mice (FIG. 5B).

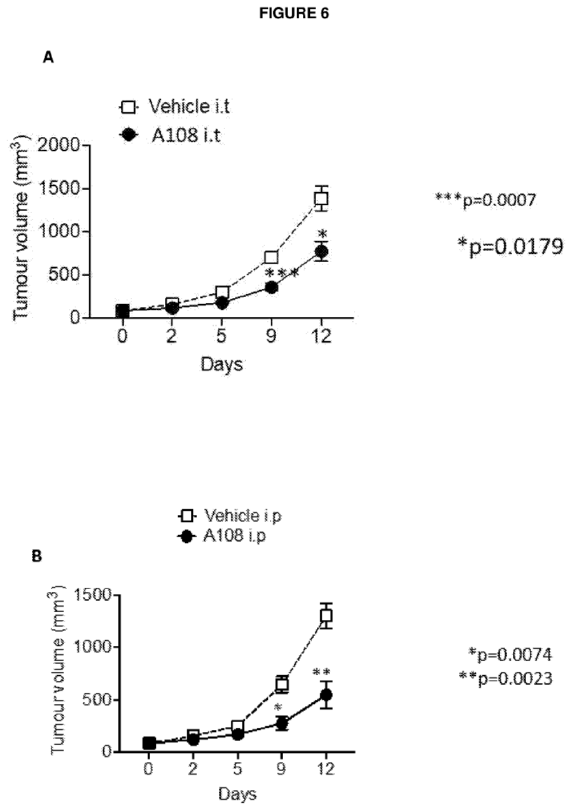

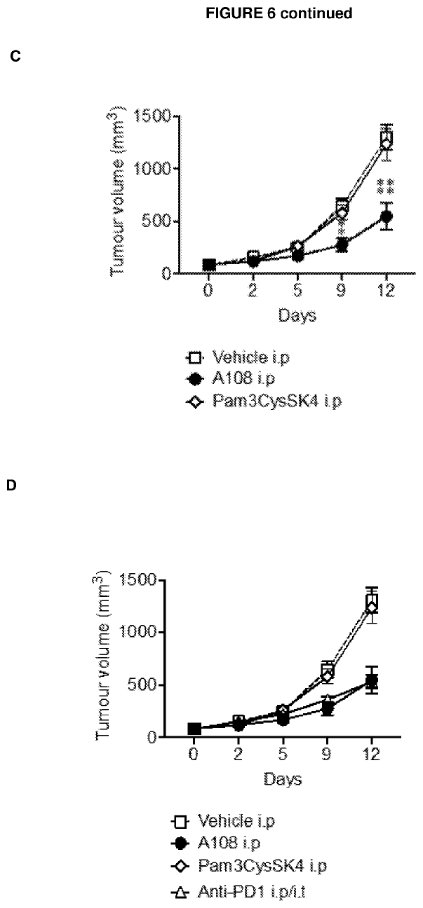

The above studies were explored further by examining the effect of compound A108 on large tumours in the MC38 mouse model. These studies aimed to understand if repeated administration over a two-week period could slow tumour growth. Mice underwent repeated dosing every 2-days for 13 days via the i.p. (10 μg dose) and i.t. (25 μg dose) routes. Mice were culled when the humane endpoint was reached or one week following the final dose (up to day 25), whichever came first. Humane endpoints included body weight loss >20% relative to weight on the first day of treatment (or weight loss >15% for 3 consecutive days), individual mouse tumour volume >3000 mm³ or the mean tumour volume of a group >2000 mm³ (all mice in the group were culled). Compound A108 slows tumour growth during the treatment phase via the i.t (FIG. 6A) or i.p (FIG. 6B) routes. Lastly, the effect of compound A108 was superior to the effect of Pam3CysSK4 (FIG. 6C), and at least as comparable as the effect of commercially available anti-PD1 antibodies (FIG. 6D).

### Example 3. Compound A108 has Anti-Tumour Activity in the WEHI164 Model

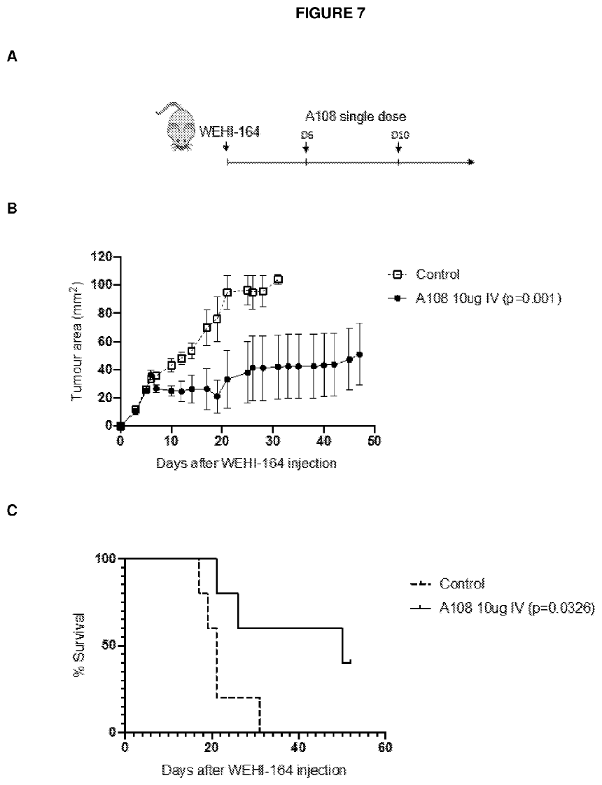

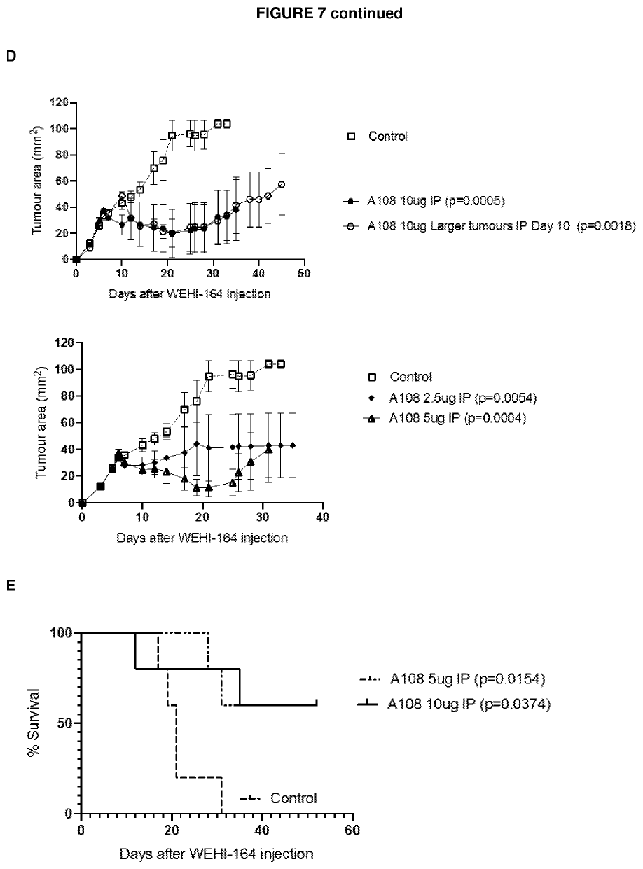

The anti-tumour effect of compound A108 was tested in the WEHI164 fibroblastoma tumour model. Balb/c mice were subcutaneously inoculated with the WEHI164 tumour cell-line and established tumours were monitored by calliper measurement. When tumours reached 30 mm² the mice were treated either via the i.v or i.p route with a single dose of compound A108 or PBS (FIG. 7A). All mice were monitored for tumour growth (FIG. 7B, D).

Groups of 5 mice were dosed with a single injection of the indicated doses per mouse. In addition, a group was injected intraperitoneally (i.p.) to test whether this route of administration could provide a quicker and safer means of drug delivery, while providing a similar therapeutic outcome. Surprisingly, the i.p. treated group produced a better rate of survival that the i.v route (FIG. 7C, E). Notably, when tumours were measured 48 hours after start of treatment, they had already responded, which is very quick for immunotherapy. Tumour regression was marked by a discolouration (blackening) of the tumour within 24 hours post treatment, exhibiting a scab on the surface in the middle, with only a small rind of tumour left, if at all (data not shown). These studies indicate that compound A108 when administered once via the i.v and i.p route is able to inhibit tumour growth.

### Example 4. Compound A108 Significantly Impairs Spontaneous Lung Metastasis in Mice Bearing EMT6.5 Tumours

Balb/c mice (n=12) were implanted orthotopically with 100,000 EMT6.5 cells into the 4^{th} inguinal mammary gland and established tumours monitored by calliper measurement. When tumours were ˜200 mm³ the mice were randomized to either one of two therapeutic arms (n=6 per arm); 1) compound A108 (10 μg, per mouse), or 2) Vehicle (saline) control. Therapy was administered intravenously to tumour bearing mice in three doses, three days apart. All mice completed therapy and were culled 21 days after tumour implant. The primary tumours, lungs and spleens were recovered and weighed. Lungs were analysed by real time quantitative PCR (RTQ-PCR) for evaluation of metastatic burden. Body weight was measured throughout the experiment.

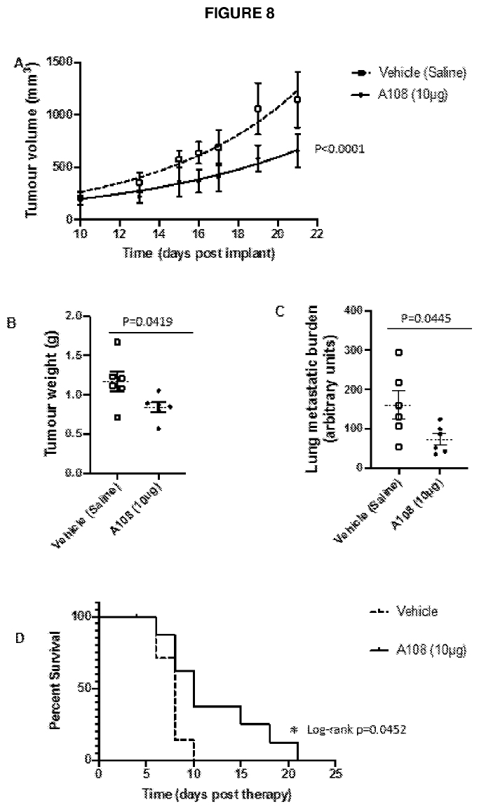

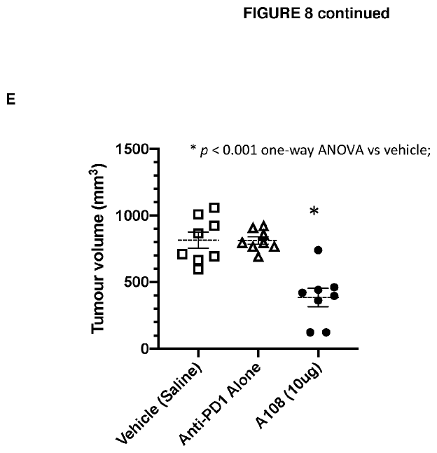

Initial compound A108 therapy induced an acute weight loss, however the weight loss decreased over subsequent dosing and it was found that it was reversible following cessation of therapy (data not shown). No severe weight loss was observed that could be attributed to any therapeutic modality however the studies showed that compound A108 significantly slowed the growth of EMT6.5 tumours in vivo (FIG. 8A).

At experimental endpoint, the primary tumour, spleens and lungs of mice were harvested to confirm reductions in primary tumour size and lung metastatic burden. As expected from the tumour growth curves, it was confirmed that compound A108 led to a statistically significant reduction in tumour weight at endpoint (FIG. 8B; tumour weight)). To quantitatively assess lung metastasis, we completed an RTQ-PCR assay to evaluate changes in lung metastatic burden. FIG. 8C demonstrates that compound A108 monotherapy led to a statistically significant reduction in spontaneous lung metastasis in mice bearing EMT 6.5 tumours. Survival data from another independent study also indicates that compound A108 can significantly prolong survival (FIG. 8D). The effect of A108 on tumour growth was significantly better than the administration of an anti PD-1 antibody, which had no effect on tumour volume (FIG. 8E).

### Example 5. Intranasal Administration of Compound A108 Reduces Lung Metastasis in Mice Bearing 4T1.2 Tumours

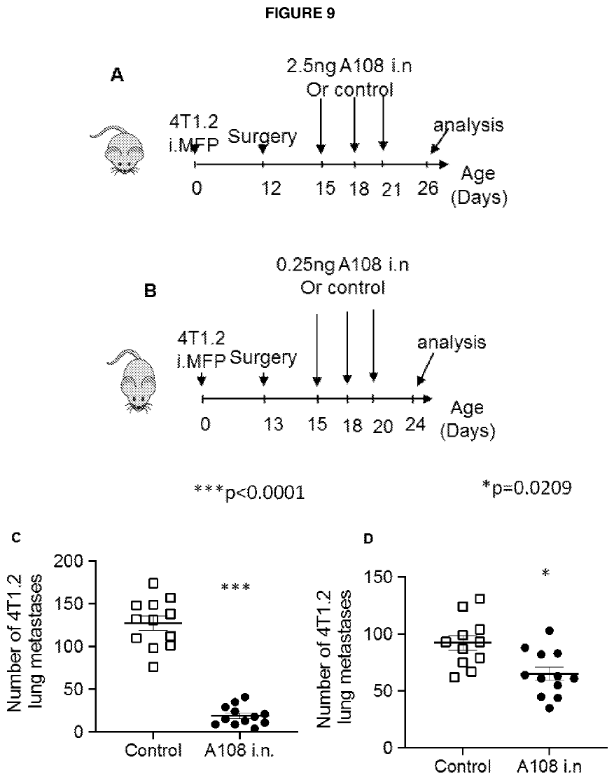

Compound A108 was next used to test its efficacy upon intranasal administration. For this, we orthotopically injected mice with highly metastatic 4T1.2 breast cancer cells into the 4^{th} mammary fat pad. Once tumours reached (˜7-8 mm in diameter), mice were subjected to surgery and the primary tumour was removed. Subsequently mice were randomized into groups receiving three intranasal administrations of either vehicle or 2.5 ng compound A108 in 50 μl saline in experiment 1 (FIG. 9A) or 0.25 ng compound A108 in 50 μl saline in experiment 2 (FIG. 9B). Two weeks after surgery all mice were sacrificed and the number of visible lung metastases were counted. In both independently performed experiments, intranasal administration of compound A108 significantly reduced the number of 4T1.2 breast cancer metastases in the lung (FIG. 9C).

### Example 6. Various Compounds Significantly Improve the Survival and Reduce Tumour Growth in MC38-Bearing Mice

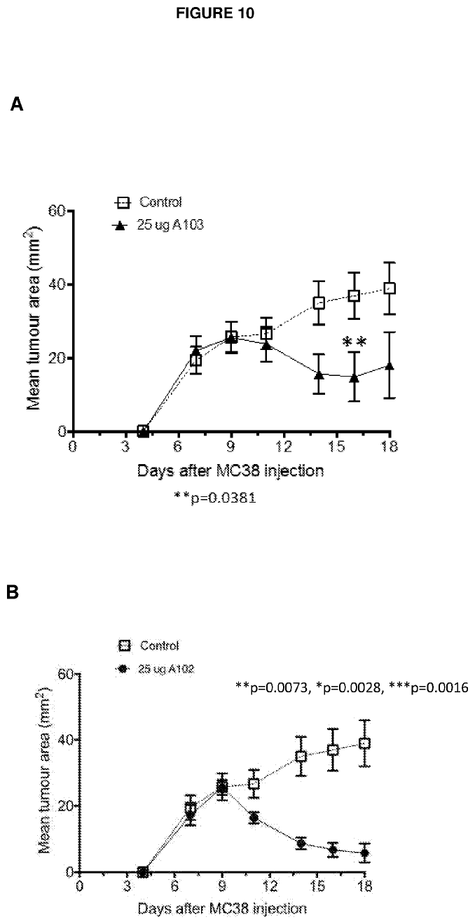

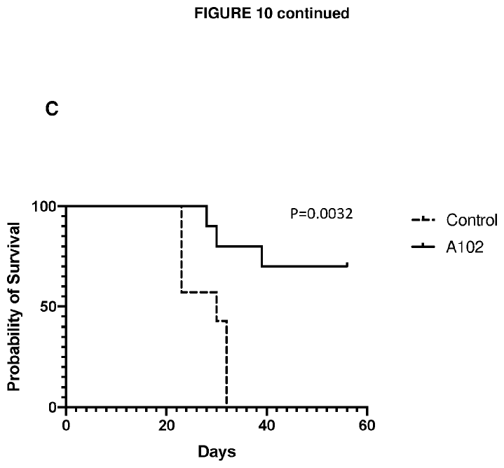

C57BL/6 WT mice were s.c injected with highly immunogenic MC38 colon carcinoma cells. Once tumours were palpable (˜5 mm in diameter), mice were randomized into two groups receiving intratumoral injections of vehicle or 25 μg of the indicated compound A102 or A103 in 100 μl saline. We observed a significant reduction in tumour growth FIG. 10A-B) and significantly improved overall survival of mice (FIG. 10C) treated with the indicated compounds, compared to vehicle control.

### Example 7. Representative Synthesis and Characterisation of Selected Compounds of the Invention: A107, A108, A115, A116, A117, A118, A203, A204, A215, A216, A220 and A224

Synthesis of Compounds A107 (x=11) and A108 (x=27)

Fmoc S-2,3-di(palmitoloxypropyl)-cysteine (S-Fmoc-Dpc-OH)_ was purchased from Bachem Inc.

Coupling of S-Fmoc-Dpc-OH to resin-bound peptide: Fmoc-Dpc-OH (100 mg, 0.24 mmol) is activated in DCM and DMF (1:1, v/v, 3 mL) with HOBt (36 mg, 0.24 mmol) and N,N′-diisopropylcarbodiimide (DICI; 37 uL, 0.24 mmol) at 0° C. for 5 min. The mixture is then added to a vessel containing the Boc-Cys-Ser(tBu)CH₂CH₂O—(PEG)₁₁-CH₂CH₂C(O)Gly resin or Boc-Cys-Ser(tBu)-CH₂CH₂O—(PEG)₂₇-CH₂CH₂C(O)Gly resin (0.25 mmol/g, 0.25 g=0.0625 mmole). After shaking for 2 h the solution is removed by filtration on a glass sinter funnel (porosity 3) and the resin washed with DCM and DMF (3×30 mL each). The reaction is monitored for completion using the trinitrobenezene sulfonic acid (TNBSA) test. If necessary a double coupling is performed.

Cleavage of peptide from the solid support: Reagent B (93% TFA, 5% water and 2% triisopropylsilane) for two hours. The peptide did not precipitate in chilled ether. Most of the TFA must be removed and then the residue is dissolved in 50% acetonitrile and purified immediately or freeze-dried.

Synthesis of Compounds A115 and A116.

Syntheses of compounds A115 (x=11) and A116 (x=27) were carried out as depicted in Scheme 2. (R)-glycidol is coupled to the thiol group of the cysteine residue attached to the peptide resin by alkylation; To 250 mg of Boc-Cys-Ser(tBu)CH₂CH₂O—(PEG)₁₁-CH₂CH₂C(O)Gly resin or Boc-Cys-Ser(tBu)CH₂CH₂O—PEG₂₇-CH₂CH₂C(O)Gly resin (0.25 mmole/g, 0.25 g=0.0625 mmole) saturated in DMF was added 250 μl of R-(+)-glycidol (MW=74.08, d=1.1, 250 μl=3.71 mmol, 60 fold excess over the free sulfhydryl group on the peptide resin) and 2511 of diisopropylethylamine (DIPEA, MW=129.2, d=0.74, 25 μl=0.14 mmol). The reaction mixture was held at 50° C. for 2 hrs in a water bath and then the solid support then thoroughly washed with DMF. To 250 mg of the peptide resin washed with toluene following glycidolation, were added 100 μl of ethylmethylsulfide (W=76.16, d=0.842, 100 μl=1.10 mmole) followed by 105 μl of tetradecyl isocyanate (MW=239, d=0.869, 105 μl=0.38 mmol, i.e. 3-fold excess over each of the hydroxyl groups present on the solid support) and finally 210 μl of dibutyltin dilaurate (MW=631.6, d=1.053, 210 μl=0.35 mmol). The reaction mixture was sparged with nitrogen gas for approximately 5 min and mixed (Intelli-Mixer, RM-2, program F26 used) overnight at room temperature. The reaction mixture was transferred to a 50 ml tube and chloroform added to 50 ml. Following sonication for approximately 5 mins the white precipitate, formed during the reaction, dissolved. The solid support was washed with DMF and acetonitrile and the final product obtained following cleavage from the support was purified by HPLC.

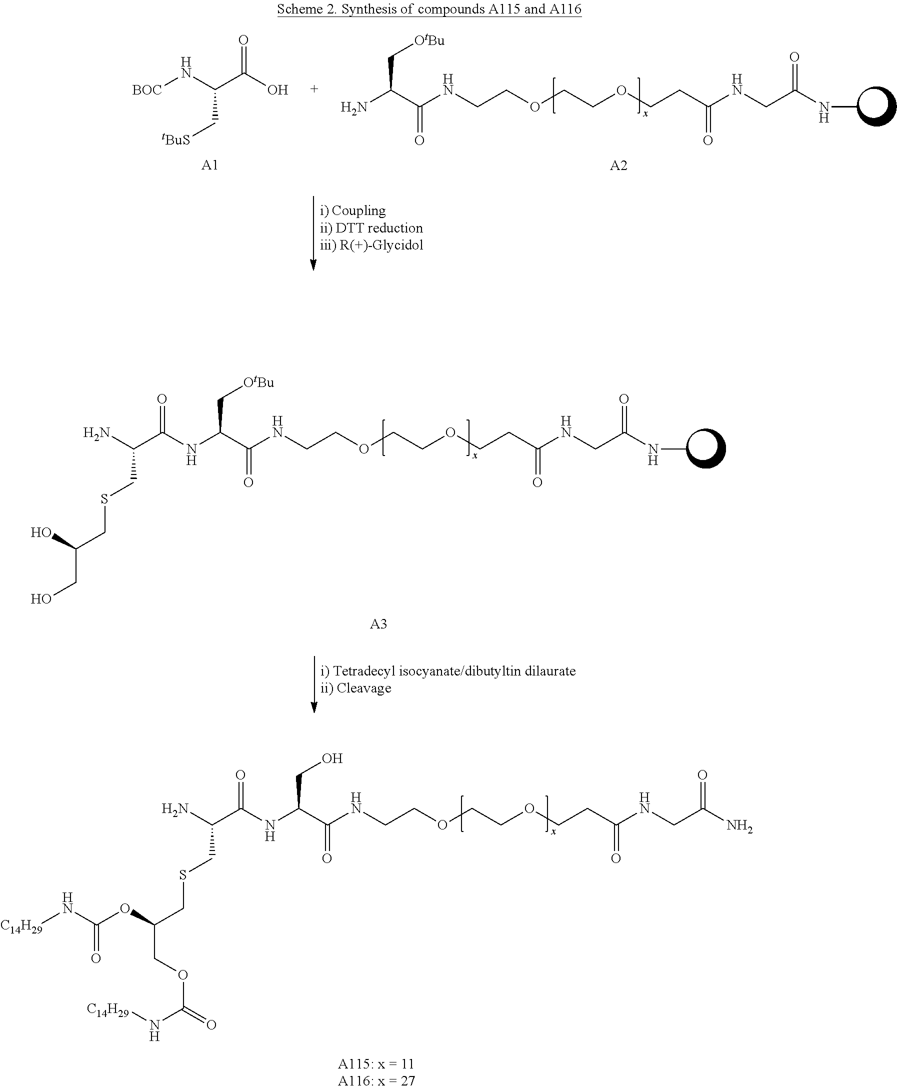

Synthesis of Compounds A117 and A118.

Compounds A117 (X═S(═O)) and A118 (X═S(═O)₂) were prepared following a similar synthetic routes as described above for compound A115, with the omission of ethylmethylsulfide scavenger, and optional omission of nitrogen sparging, from the carbamate formation step. Omitting the ethylmethylsulfide scavenger yielded a mixture of compounds A115, A117 and A118 which were separated and purified by HPLC.

Alternatively, sulfone or sulfoxide derivatives (eg A117 and A118) may be prepared by oxidation of the corresponding sulfide (eg A115) with an oxidant such as meta-chloroperoxybenzoic acid (MCPBA) or tert-butyl hydroperoxide (t-BuOOH) under appropriate conditions.

Synthesis of Compounds A203 and A204.

The synthesis of compounds A203 and A204 is depicted below in Scheme 3.

Fmoc-Gly was added as the first amino acid to the solid support, followed by coupling of Fmoc-NHCH₂CH₂O—(PEG)₁₁-CH₂CH₂COOH or Fmoc-NHCH₂CH₂O—(PEG)₂₇-CH₂CH₂COOH in 2-fold molar excess in presence of a two-fold excess of Hexafluorophosphate Benzotriazole Tetramethyl Uronium (HBTU), Hydroxybenzotriazole (HOBT) and 4-fold excess of diisopropylethylamine (DIPEA) in 2 ml of dimethylformamide (DMF) for 2 hrs. Fmoc-Ser(tBu)-OH is then coupled to provide intermediate A2, followed by the coupling of Boc-Cys(StBu) A1. The thiol-tert-butyl group on the cysteine residue was removed by incubating the peptide resin in 0.5M of dithiothreitol for 1 hr in DMF at RT. To 250 mg of Boc-Cys-Ser(tBu)-NHCH₂CH₂O—(PEG)₁₁-CH₂CH₂C(O)Gly resin or Boc-Cys-Ser(tBu) CH₂CH₂O—(PEG)₂₇-CH₂CH₂C(O)-Gly resin (0.25 mmole/g, 0.25 g=0.0625 mmole) saturated in DMF was added 250 μl of R-(+)-1,2-epoxy-butan-4-ol [(R)-2-(oxiran-2-yl)ethan-1-ol] (Mw=88.11, d=1.1, 250 μl=3.125 mmol equivalent to a 50 fold excess over the free sulfhydryl group present on the peptide resin) and 25 μl of diisopropylethylamine (DIPEA, Mw=129.2, d=0.74, 25 μl=0.14 mmol). The reaction mixture was left in a water bath at 50° C. for 2 hrs and then thoroughly washed with DMF to provide intermediate A3.

Palmitic acid (320 mg, 1.25 mmol), DIPCDI (225 uL, 1.5 mmol) and 4-dimethylaminopyridine (DMAP; 15.25 mg, 0.125 mmol) were dissolved in 2 mL of dichloromethane (DCM) then added to the resin-bound BOC-Dhc-peptide resin A3 (0.0625 mmol, 0.25 g) and shaken for 16 h at room temperature. The supernatant was removed by filtration and the solid support thoroughly washed with DCM and dimethylformamide (DMF) to remove any residue of urea before being subjected to the cleavage process as described below.

The solid support bearing the assembled lipopeptide was exposed to reagent B (93% TFA, 5% water and 2% triisopropylsilane) for 2 hours. To isolate the product, most of the TFA was removed and the residue is then dissolved in 50% acetonitrile and purified immediately using the purification protocol described below or the material was freeze-dried and stored for later purification.

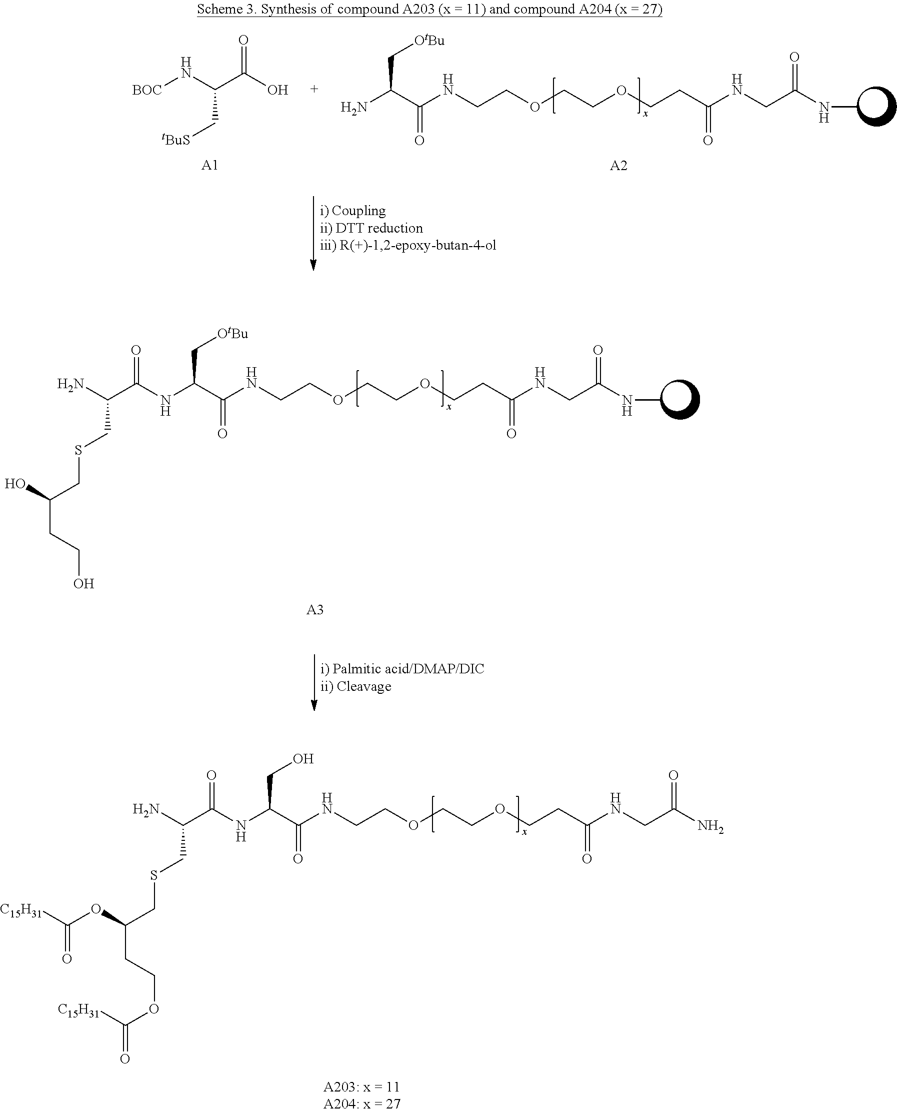

Synthesis of Compound A215 and Compound A216.

The synthesis of compounds A215 and A216 was carried out as depicted in Scheme 4. Intermediate A3 was prepared as described for compounds 3 and 4 above.

Then, to 250 mg of the peptide resin washed with toluene following glycidolation, were added 100 μl of ethylmethylsulfide (Mw=76.16, d=0.842, 100 μl=1.10 mmol) followed by 105 μl of tetradecyl isocyanate (MW=239, d=0.869, 105 μl=0.38 mmol, i.e. 3-fold excess over each of the hydroxyl groups present on the solid support) and finally 210 μl of dibutyltin dilaurate (Mw=631.6, d=1.053, 210 μl=0.35 mmol). The reaction mixture was sparged with nitrogen gas for approximately 5 min and mixed (Intelli—Mixer, RM-2, program F26 used) overnight at room temperature. The reaction mixture was transferred to a 50 ml tube and chloroform added to 50 ml. Following sonication for approximately 5 mins the white precipitate, formed during the reaction, dissolved. The solid support was washed with DMF and acetonitrile and the final product obtained following cleavage (as above) from the support was purified by HPLC.

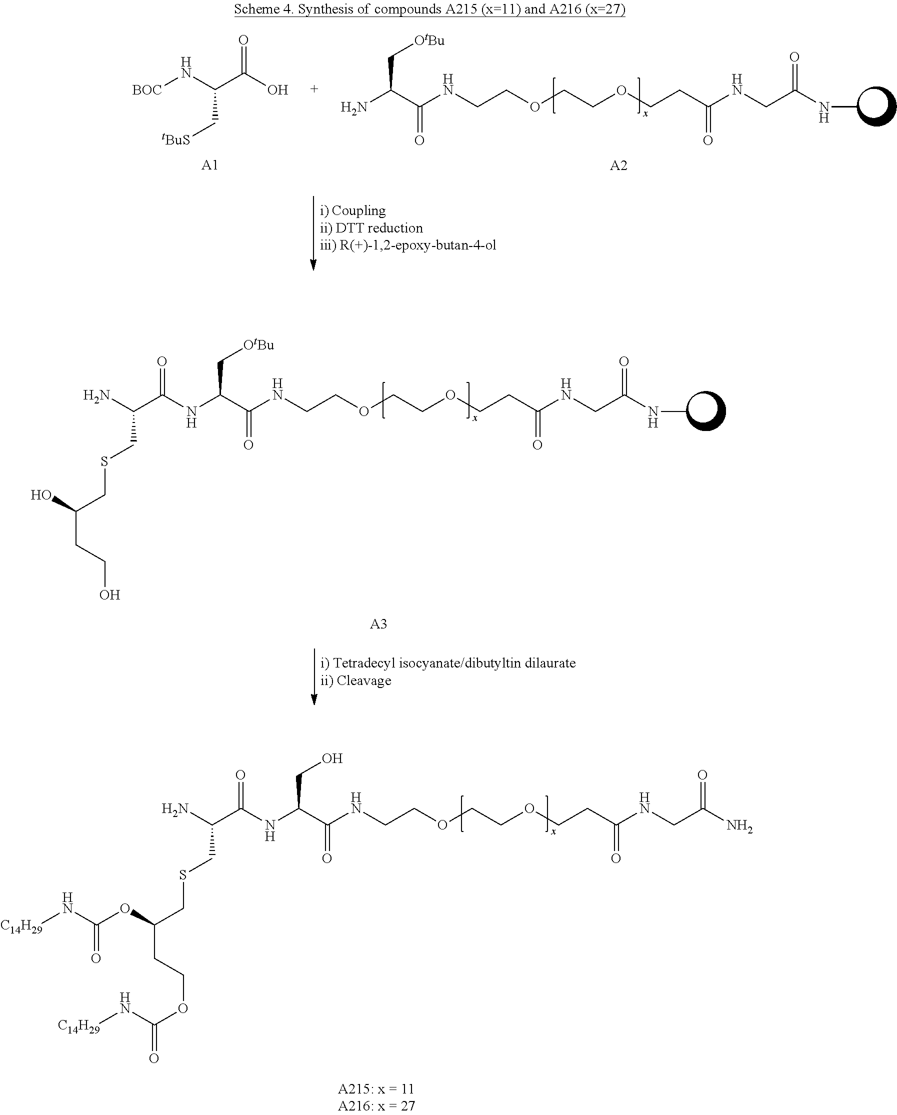

Synthesis of A220. Compound A220 was synthesized by standard Fmoc Solid Phase Peptide Synthesis, starting with Fmoc-RINK MBHA PS Resin. Removal of the Fmoc group after each coupling was achieved using 20% piperidine in DMF. Couplings of Fmoc-Gly-OH (2-fold excess), Fmoc-NH-PEG₂₈-CH₂CH₂COOH (1.4-fold excess), Fmoc-Ser(tBu)-OH (2-fold excess), and N-(Boc)-S—((R)-2,3-dihydroxybutyl)-L-cysteine (1.5-fold excess) were performed in DMF using equivalent excess of ethyl cyano(hydroxyimino)acetate (Oxyma Pure) and diisopropylcarbodiimide (DIC) as coupling agents. Myristyl Chloroformate coupling was performed using Myristyl Chloroformate (12 eq. vs. moles resin), DIEA (24 eq. vs. moles resin) in dry DCM for 18 hours at room temperature. This coupling was repeated three times (“recoupling”). The first recoupling was done using Myristyl Chloroformate (12 eq. vs. moles resin), NMM (24 eq. vs. moles resin) in dry DCM/THF (85/15) for 18 hours at room temperature. The second recoupling was done using Myristyl Chloroformate (6 eq. vs. moles resin), NMM (12 eq. vs. moles resin) in dry DCM/THF (85/15) for 41 hours at room temperature. Finally the third recoupling was performed using Myristyl Chloroformate (6 eq. vs. moles resin), NMM (12 eq. vs. moles resin) in dry DCM/THF/Toluene (85/15/5) for 21.5 hours at room temperature.

Cleavage of the peptide from the resin, removal of N-terminal Boc group, and serine side-chain deprotection were achieved by exposure of the resin to a solution of 93% trifluoroacetic acid (TFA), 5% H₂O, 3% triisopropylsilane (TIPS) for 1.5 hours. Following the cleavage reaction, the mixture was evaporated and the resulting residue was re-dissolved in 30% acetonitrile/water and lyophilized.

Synthesis of A224. Compound A224 was synthesized by standard Fmoc Solid Phase Peptide Synthesis, starting with Chlorotrityl Chloride Resin with an initial substitution of 1.6 meq/g. The first amino acid, Fmoc-Gly-OH, was loaded on the resin first, using a 0.5-fold molar excess of Fmoc-Gly-OH and DIEA (1.5-fold excess), followed by capping with DMF/MeOH/DIEA (80/10/10), and Fmoc deprotection, to obtain the dry loaded H-Gly-CT Resin with a final substitution of 0.67 meq/g. Removal of the Fmoc group after each coupling was achieved using 20% Piperidine in DMF. Coupling of Fmoc-NH-PEG₂₈-CH₂CH₂COOH (1.4 eq.) was performed using (7-Azabenzotriazol-1-yloxy)trispyrrolidinophosphonium hexafluorophosphate (PyAOp; 1.4 eq.), diisopropylethylamine (DIEA; 3.2 eq.) in DMF, whereas couplings of Fmoc-Ser(tBu)-OH (2eq), and N-(Boc)-S—((R)-2,4-dihydroxybutyl)-L-cysteine (1.5 eq.) were performed in DMF using equivalent excess of Oxyma Pure and DIC as coupling agents. Palmitic Acid coupling was performed using palmitic acid (20 eq. vs. moles resin), DIC (20 eq.), DMAP (2eq.) in DCM/THF (85/15) (v/v) for 24 hours at room temperature.

Cleavage of the peptide from the resin, removal of N-terminal Boc group, and serine side-chain deprotection were achieved by exposure of the resin to a solution of 93% TFA, 5% H2O, 3% TIPS for 1.5 hours. Following the cleavage reaction, the mixture was evaporated and the resulting residue was re-dissolved in 30% Acetonitrile/Water and lyophilized.

Purification and Characterisation

Purification and characterisation: Following cleavage from the solid support, each of the analogs were purified by reversed-phase HPLC according to either protocol A or B described below.

Protocol A: Reversed phase HPLC was conducted using an Agilent Zorbax 300SB-C3, 5 um column (9.4 mm×250 mm; Agilent Technology, Australia) installed in an Agilent HPLC 1260 Infinity system (Agilent Technologies, Santa Clara, Calif., USA) with the chromatogram developed using Buffer A (0.1% trifluoroacetic acid in water) and buffer B (0.1% trifluoroacetic acid in acetonitrile).

Protocol B: Reverse phase chromatography was conducted using a Novasep Axial Compression Column (5-cm diameter) loaded with cyano media (Daisogel SP-120-CN-P), with a gradient of Acetonitrile in [0.1% TFA/Water]. Following intermediate lyophilization, ion-exchange was performed on Dowex ion-exchange resin in order to obtain the peptide as the acetate salt.

Identification and purity determination of the target materials were carried out using an in-line HPLC-MS system using the following conditions:

Conditions A: HPLC column: Agilent Zorbax 300-SB C3 (150×0.5 mm; 5 μm) with the following gradient conditions: 0-5 min, 20% B: 5-32 min, 20% B-100% B: 32-40 min, 100% B-20% B. The flow rate was 20 μl/min. LC-MS: Agilent 1100 series capillary LC system in-line with an Agilent 1100 series LC/MSD ion-trap mass spectrometer. The mass spectrometer was operated with electrospray ionisation configured in the positive ion mode. Data analysis software from Agilent Technologies was used to de-convolute the charged ion series for identification of the peptide material and the material then characterised by LC-MS.

Conditions B: analytical reverse phase HPLC with a cyano column (Daiso Fine Chem, SP-120-3-CN-P, 150×4.6 mm, 3 μm, 120 Å). The peptide was also analyzed by ESI LC-MS in Positive Ion Mode, using a Finnigan LCQ Deca XPMax.

Compounds A107, A108, A115, A116, A203, A204, A215 and A216 following protocol A and conditions A, compounds A220 and A224 were prepared and purified as described above following protocol B and conditions B, and prepared and purified as described above, were each found to be greater than 95% pure.

Peptide Quantitation

Quantitation of compounds A107, A108, A115, A116, A203, A204, A215 and A216 was carried out by in vacuo hydrolysis at 110° C. of samples in sealed glass vials in the presence of 6N HCl containing 0.1% phenol. Derivatisation of amino acids was then carried out using Waters AccQTag reagents according to the manufacturer's instructions followed by analysis on a Waters Acquity UPLC System (Waters Millipore) using an AccQTag ultra column (2.1 mm×100 mm; Waters Millipore). Quantitation of other compounds may be achieved by a similar protocol.

Synthesis of Sulfone and Sulfoxide Analogues of Compounds A215 and A216

Sulfone and sulfoxide derivatives of compounds A215 and A216 may be accessed by a similar synthetic routes as described above, with the omission of ethylmethylsulfide scavenger, and optional omission of nitrogen sparging, from the carbamate formation step. This reaction may yield a mixture of thiol, sulfone and sulfoxide derivatives, which may be separated and purified by HPLC.

Alternatively, sulfone or sulfoxide derivatives may be prepared by oxidation of the corresponding sulfide with an oxidant such as meta-chloroperoxybenzoic acid (MCPBA) or tert-butyl hydroperoxide (t-BuOOH) under appropriate conditions.

1. A method of treating, preventing or minimising progression of cancer in a subject comprising administering a therapeutically effective amount of a compound of formula (I):
A-Y—B (I)
wherein A comprises or consists of a moiety selected from A1 and A2: 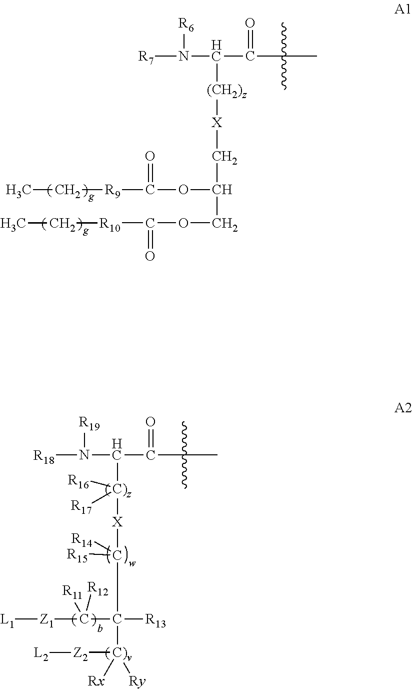
wherein
each z is independently selected from 1 or 2;
each X is independently selected from —S—, —S(═O)— and —S(═O)₂—;
in moiety A1:
each g is independently 10, 11, 12, 13, 14, 15, 16, 17 or 18;
R₆ and R₇ are independently selected from the group consisting of H, a straight or branched C₁-C₄ alkyl, and —C(═O)CH₃;
R₉ and R₁₀ are independently selected from the group consisting of —NH—, —O— or a single bond; and
in moiety A2:
b and w are each independently an integer from 0 to 7 and v is an integer from 0 to 5, such as from 2 to 5, provided that:
the sum of b, v, and w is at least 3; and
the sum of b and w is from 0 to 7;
Z₁ and Z₂ are each independently selected from the group consisting of —O—, —NR—, —S—, S(═O), —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; Y is 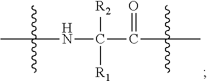
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₃ is selected from the group consisting of H and a straight or branched C₁-C₆ alkyl; and
B is polyethylene glycol (PEG),
or a pharmaceutically acceptable salt, solvate or prodrug thereof.

2. A method according to claim 1, wherein
A comprises or consists of moiety A1; and
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H.

3. A method according to claim 1, wherein
A comprises or consists of moiety A1;
R₆ and R₇ are H;
R₉ and R₁₀ are both a single bond;
R₈ is selected from the group consisting of H and a straight or branched C₁-C₆ alkyl;
z is 1; and
X is S.

4. A method according to claim 1, wherein
A comprises or consists of moiety A1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are H;
R₉ and R₁₀ are both a single bond;
z is 1; and
X is S.

5. A method of treating, preventing or minimising progression of cancer in a subject comprising administering a therapeutically effective amount of a compound comprising a moiety A selected from A1′ and A2 as defined in claim 1 and PEG, wherein the moiety A and PEG are linked by a glycine, serine, homoserine, threonine, phosphoserine, asparagine or glutamine residue, or an ester of a glutamine residue.

6. A method according to claim 5, wherein the moiety A and PEG are linked by a serine, homoserine, threonine or phosphoserine residue.

7. A method of treating, preventing or minimising progression of cancer in a subject comprising administering a therapeutically effective amount of a compound of formula (VIII):
A-Y—NH—(CH₂)ₚ—O—(CH₂—CH₂—O)—[(CH₂)ₘCO-L-]_{q}R₃ (VIII)
wherein
A is a moiety selected from A1 and A2 as defined in claim 1
Y is 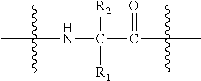
n is 3 to 100;
m is 1, 2, 3 or 4;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 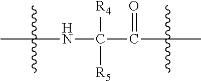
wherein R₄ is H; and
R₅ is the side chain, or second hydrogen of the amino acid
or a pharmaceutically acceptable salt, solvate or prodrug thereof.

8. A method according to claim 7, wherein
A comprises or consists of moiety A1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H.

9. A method according to claim 7, wherein
A comprises or consists of moiety A1;
R₆ and R₇ are H;
R₈ is selected from the group consisting of H and a straight or branched C₁-C₆ alkyl;
R₉ and R₁₀ are both a single bond;
z is 1; and
X is S.

10. A method according to claim 7, wherein
A comprises or consists of moiety A1;
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H;
R₆ and R₇ are H;
R₉ and R₁₀ are both a single bond;
z is 1; and
X is S.

11. A method of treating, preventing or minimising progression of cancer in a subject comprising administering a therapeutically effective amount of a compound of formula (VIII):
Pam2Cys-Y—NH—(CH₂)ₚ—O—(CH₂—CH₂-)ₙ-[(CH₂)ₘCO-L-]_{q}R₃ (X)
wherein
Pam2Cys has the structure: 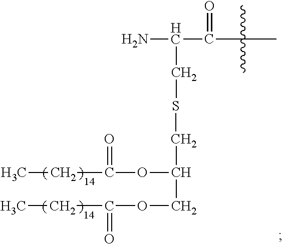
Y is 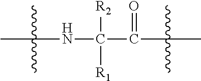
n is 3 to 100;
m is 1, 2, 3 or 4;
p is 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₈ is selected from the group consisting of H and a straight or branched C₁-C₆ alkyl;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 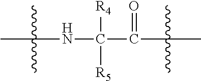
wherein R₄ is H; and
R₅ is the side chain, or second hydrogen of the amino acid,
or a pharmaceutically acceptable salt, solvate or prodrug thereof.

12. A method according to claim 11, wherein
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H.

13. A method of treating, preventing or minimising progression of cancer in a subject comprising administering a therapeutically effective amount of a compound of formula (IV):
Pam2Cys-Ser-NH—(CH₂)ₚ—O—(CH₂—CH₂—O)ₙ—[(CH₂)ₘCO-L-]_{q}R₃ (XIII)
wherein
Pam2Cys-Ser has the structure: 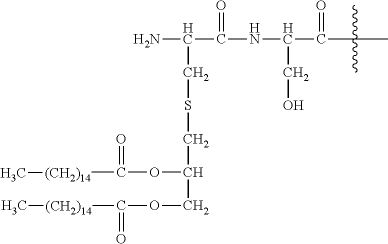
n is 3 to 100;
m is 1, 2, 3 or 4;
p is 2, 3 or 4;
q is null or 1;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 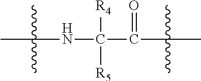
wherein R₄ is H; and
R₅ is the side chain, or second hydrogen of the amino acid,
or a pharmaceutically acceptable salt, solvate or prodrug thereof.

14. A method of treating, preventing or minimising progression of cancer in a subject comprising administering a therapeutically effective amount of a compound of formula (V): 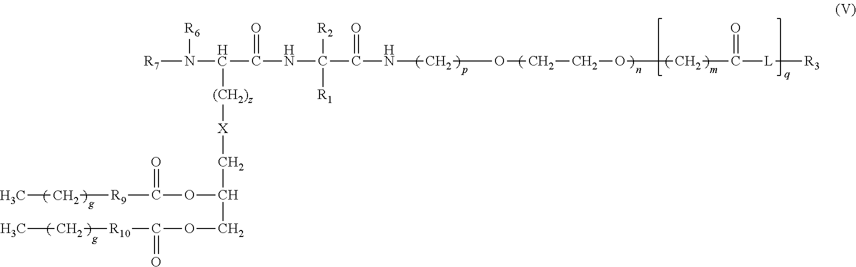
wherein
n is 3 to 100;
m is 1, 2, 3 or 4;
each g is independently 10, 11, 12, 13, 14, 15, 16, 17 or 18;
p is 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 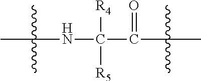
wherein R₄ is H; and
R₅ is the side chain, or second hydrogen of the amino acid,
or a pharmaceutically acceptable salt, solvate or prodrug thereof.

15. A method according to claim 14, wherein
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H.

16. A method according to claim 14 or 15, wherein
R₆ and R₇ are H;
R₈ is selected from the group consisting of H and a straight or branched C₁-C₆ alkyl;
R₉ and R₁₀ are both a single bond;
z is 1; and
X is S.

17. A method of treating, preventing or minimising progression of cancer in a subject comprising administering a therapeutically effective amount of a compound of formula (VII): 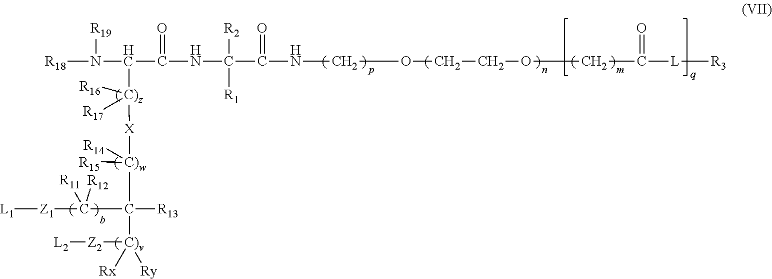
wherein
n is 3 to 100;
m is 1, 2, 3 or 4;
p is 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and CH₂CH₂C(═O)OR₈, wherein any one of the alkyl hydrogens can be replaced with a halogen;
R₈ is selected from the group consisting of H and a straight or branched C₁-C₆ alkyl;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 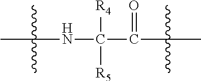
wherein R₄ is H; and
R₅ is the side chain, or second hydrogen of the amino acid;
b and w are each independently an integer from 0 to 7 and v is an integer from 0 to 5, provided that:
the sum of b, v, and w is at least 3; and
the sum of b and w is from 0 to 7;
z is 1 or 2;
X is selected from —S—, —S(═O)— and —S(═O)₂—;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A₂;
L₁ and L₂ are each independently C₅-C₂₁ aliphatic or C₄-C₂₀ heteroaliphatic;
L₃ is C₁-C₂₁ aliphatic or C₂-C₂₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted;
or a pharmaceutically acceptable salt, solvate or prodrug thereof.

18. A method of treating, preventing or minimising progression of cancer in a subject comprising administering a therapeutically effective amount of a compound of formula (XVII): 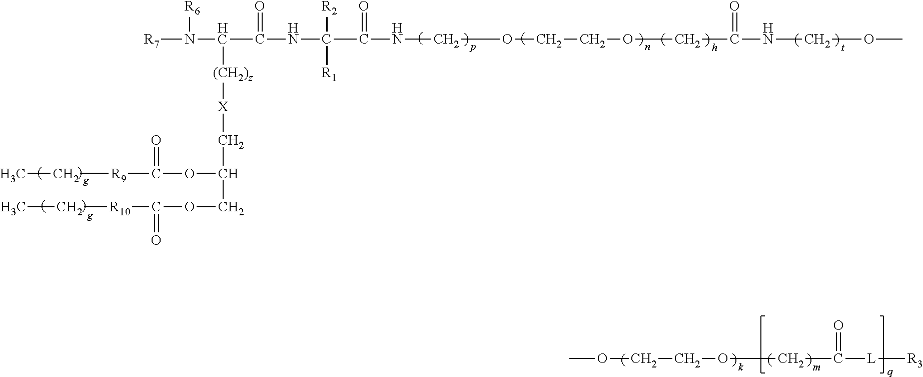
wherein
n is 3 to 100;
k is 3 to 100;
m is 1, 2, 3 or 4;
each g is independently 10, 11, 12, 13, 14, 15, 16, 17 or 18;
p is 2, 3 or 4;
t is 2, 3 or 4;
h is 1, 2, 3 or 4;
q is null or 1;
R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
wherein when q=1, R₃ is —NH₂ or —OH;
wherein when q=0, R₃ is H;
L is null or consists of 1 to 10 units, wherein each unit is a natural alpha amino acid or derived from a natural alpha amino acid, and has the formula: 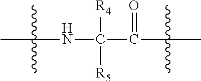
wherein R₄ is H; and
R₅ is the side chain, or second hydrogen of the amino acid,
or a pharmaceutically acceptable salt, solvate or prodrug thereof.

19. A method of claim 18, wherein
R₁ and R₂ are independently selected from the group consisting of H, —CH₂OH, —CH₂CH₂OH, —CH(CH₃)OH and —CH₂OPO(OH)₂, wherein any one of the alkyl hydrogens can be replaced with a halogen, and wherein R₁ and R₂ are not both H.

20. A method of claim 18 or 19, wherein
R₆ and R₇ are H;
R₈ is selected from the group consisting of H and a straight or branched C₁-C₆ alkyl;
R₉ and R₁₀ are both a single bond;
z is 1; and
X is S.

21. A method according to any one of claims 1, 5-7 and 17, wherein
v is 2;
b is 0;
w is 1;
the sum of v, b and w is 3;
the sum of b and w is 1;
z is 1;
X is S
Z₁ and Z₂ are independently selected from the group consisting of —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each H;
R and R₁₃ are each H;
R₁₈ is H;
R₁₉ is selected from the group consisting of H, C₁-C₆ alkyl, —C(═O) C₁-C₆ alkyl or —C(═O)C₁₁-C₁₉alkyl; and
L₁ and L₂ and independently selected from C₁₀-C₁₈ aliphatic or C₁₀-C₁₈ heteroaliphatic.

22. A method according to any one of claims 7 to 21, wherein q is 1.

23. A method according to any one of claims 1 to 10, 14 to 16 and 18 to 20, wherein
g is an integer from 12 to 16.

24. A method according to claim 23, wherein g is 14.

25. A method according to any one of claims 7 to 24, wherein n is an integer from 10 to 14.

26. A method according to claim 25, wherein n is 11.

27. A method according to any one of claims 7 to 24, wherein n is between 24 and 30.

28. A method according to claim 27, wherein n is 27.

29. A method according to any one of claims 7 to 28, wherein m is 1-3.

30. A method according to claim 29, wherein m is 2.

31. A method of treating, preventing or minimising progression of cancer in a subject comprising administering a therapeutically effective amount of a compound comprising or consisting of partial structure A1Y′ or A2Y′: 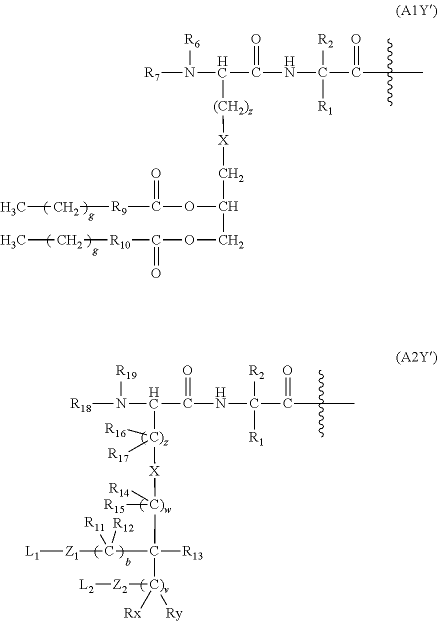
wherein R₁ and R₂ are independently selected from the group consisting of H, —CH₂OH, —CH₂CH₂OH, —CH(CH₃)OH, —CH₂OPO(OH)₂, —CH₂C(═O)NH₂, —CH₂CH₂C(═O)OH and —CH₂CH₂C(═O)OR₈, wherein any one of the alkyl hydrogens can be replaced with a halogen;
R₆ and R₇ are independently selected from the group consisting of H, a straight or branched C₁-C₄ alkyl, and —C(═O)CH₃;
R₈ is selected from the group consisting of H and a straight or branched C₁-C₆ alkyl;
R₉ and R₁₀ are independently selected from the group consisting of —NH—, —O— or a single bond;
z is 1 or 2;
X is selected from —S—, —S(═O)— and —S(═O)₂—;
b and w are each independently an integer from 0 to 7 and v is an integer from 0 to 5, provided that:
the sum of b, v, and w is at least 3; and
the sum of b and w is from 0 to 7;
Z₁ and Z₂ are each independently selected from the group consisting of —O—, —NR—, —S—, —S(═O)—, —S(═O)₂—, —C(═O)O—, —OC(═O)—, —C(═O)NR—, —NRC(═O)—, —C(═O)S—, —SC(═O)—, —OC(═O)O—, —NRC(═O)O—, —OC(═O)NR—, and —NRC(═O)NR—;
R₁₁, R₁₂, Rₓ, R_{y}, R₁₄, R₁₅, R₁₆, and R₁₇ at each instance of b, v, w, and z are each independently H or C₁-C₆ aliphatic;
R, R₁₃ and R₁₈ are each independently H or C₁-C₆ aliphatic;
R₁₉ is H, C₁-C₆ aliphatic, an amino protecting group, L₃-C(═O)—, or A2;
L₁ and L₂ are each independentlyC₅-C₂₁ aliphatic or C₄-C₂₀ heteroaliphatic;
L₃ is C₁-C₂₁ aliphatic or C₂-C₀ heteroaliphatic;
A₂ is an amino acid or a peptide;
wherein any aliphatic or heteroaliphatic present in any of R, R₁₁, R₁₂, R₁₃, R₁₄, R₁₅, R₁₆, R₁₇, R₁₈, R₁₉, Rₓ, R_{y}, L₁, L₂, and L₃ is optionally substituted; and
A1Y′ or A2Y′ is covalently linked to polyethylene glycol (PEG),
or a pharmaceutically acceptable salt, solvate or prodrug thereof.

32. A method according to any one of the preceding claims, or a pharmaceutically acceptable salt, solvate or prodrug thereof, wherein the compound is the R diastereomer of the compound around the following chiral centre:

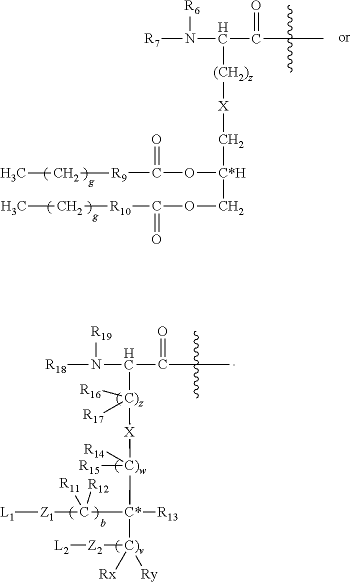

33. A method according to any one of the preceding claims, or a pharmaceutically acceptable salt, solvate or prodrug thereof, wherein the compound is the L-diastereomer of the compound around the chiral centre denoted * or **:

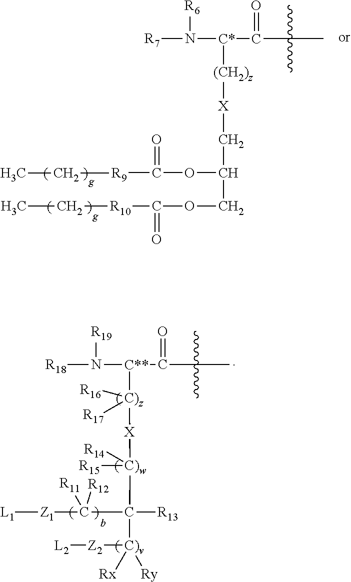

34. A method according to any one of the preceding claims, or a pharmaceutically acceptable salt, solvate or prodrug thereof, wherein the compound is the L-diastereomer of the compound around the following chiral centre:

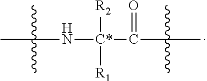

35. A method of treating, preventing or minimising progression of cancer in a subject comprising administering a therapeutically effective amount of a compound selected from any one of the group consisting of:

| Compound Structure | Compound ID |
|---|---|
| 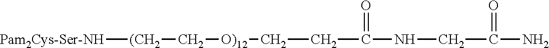 | A101/ compound 1 |
| 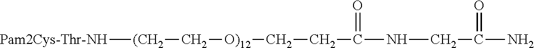 | A102 |
| 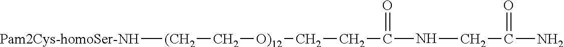 | A103 |
| 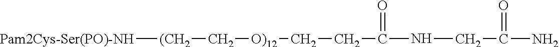 | A104 |
| 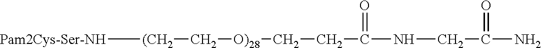 | A105 |
| 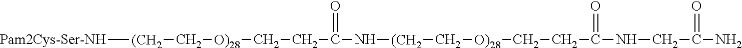 | A106 |
| 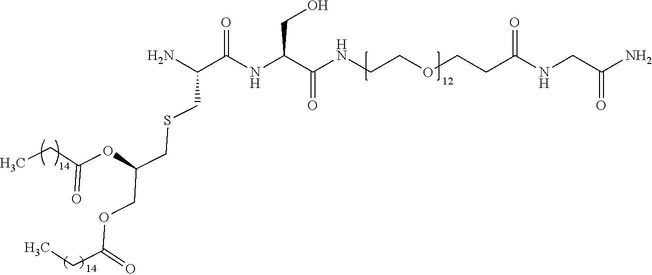 | A107 |
| 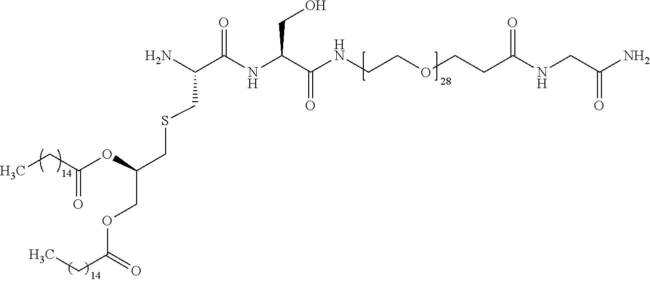 | A108 |
| 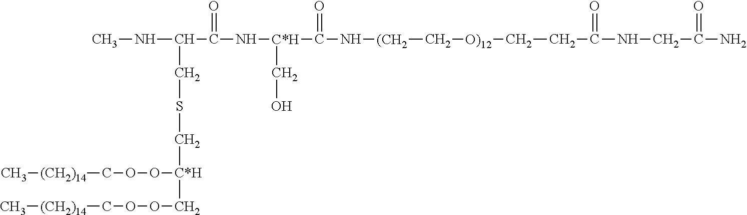 | A109 |
| 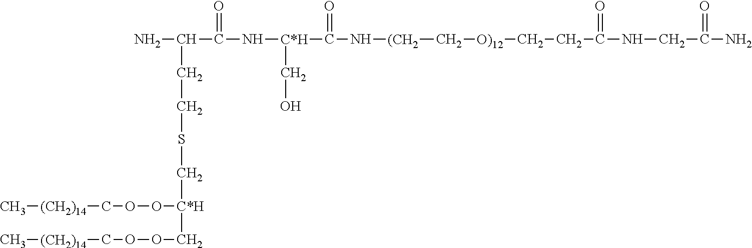 | A110 |
| 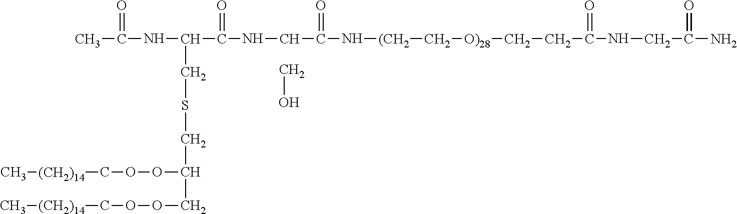 | A111 |
| 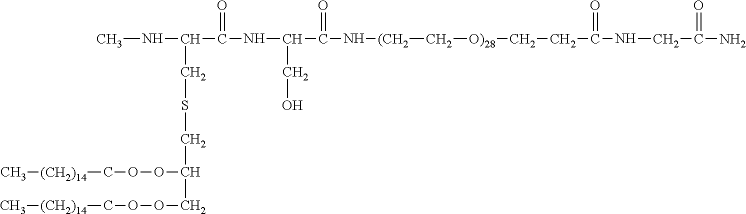 | A112 |
| 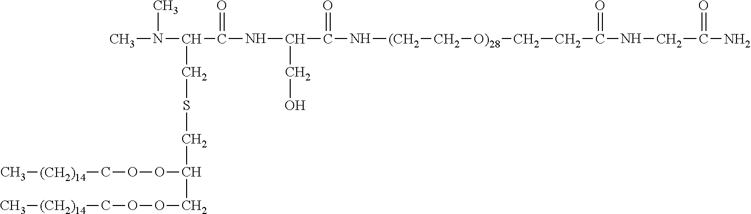 | A113 |
| 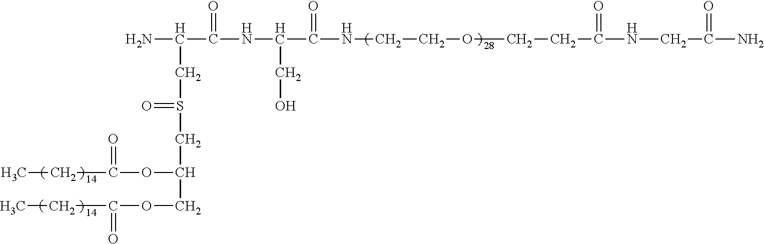 [SMILES: CCC(=O)OCC(CS(=O)CC(N)C(=O)NC(CO)C(=O)NCCOCCC(=O)NCC(N)=O)OC(=O)CC] | A114 |
| 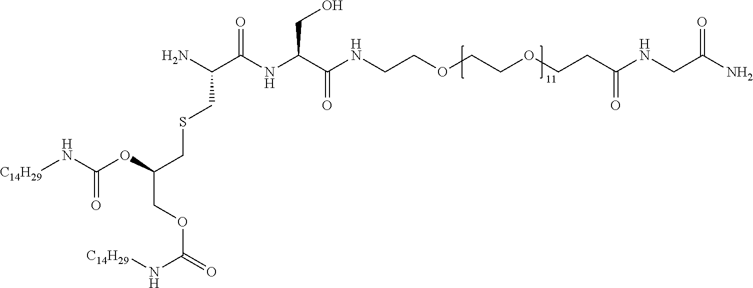 | A115 |
| 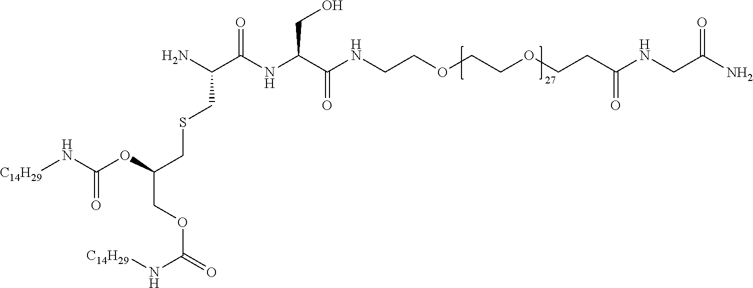 | A116 |
| 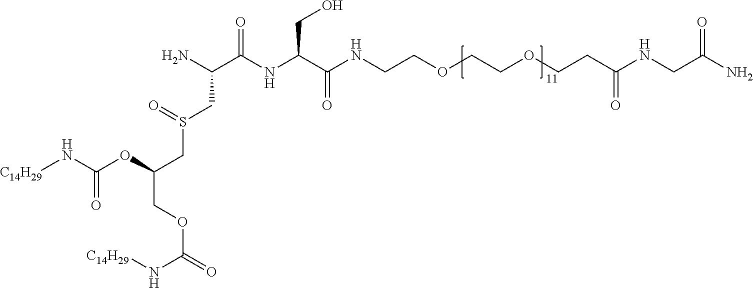 | A117 |
| 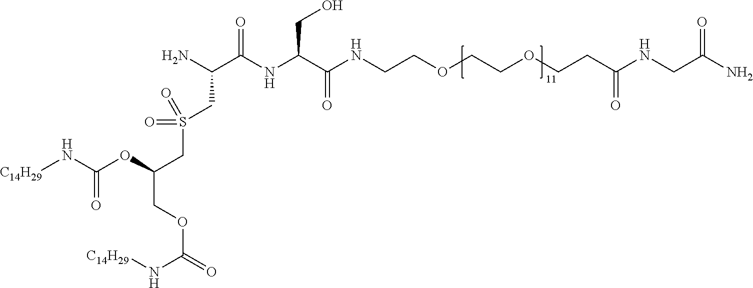 | A118 |
| 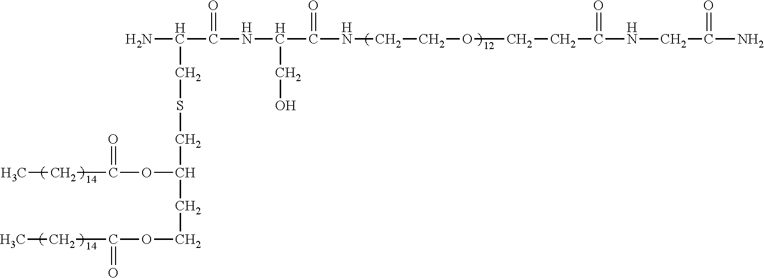 [SMILES: CCC(=O)OCCC(CSCC(N)C(=O)NC(CO)C(=O)NCCOCCC(=O)NCC(N)=O)OC(=O)CC] | A201 |
| 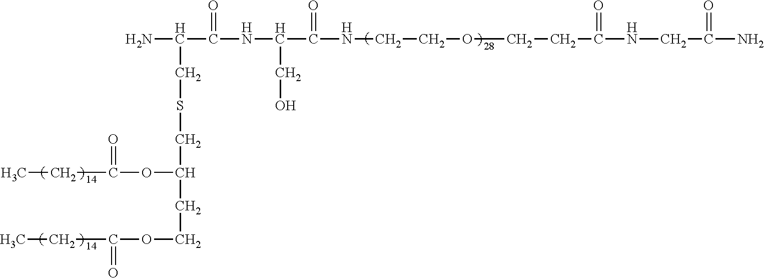 [SMILES: CCC(=O)OCCC(CSCC(N)C(=O)NC(CO)C(=O)NCCOCCC(=O)NCC(N)=O)OC(=O)CC] | A202 |
| 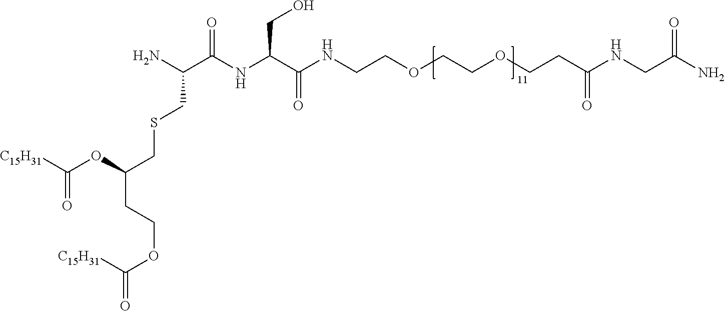 | A203 |
| 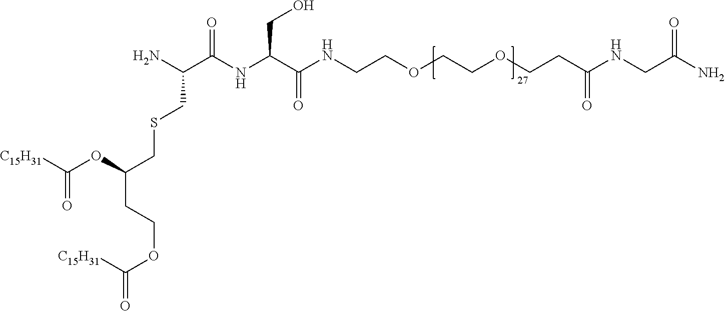 | A204 |
| 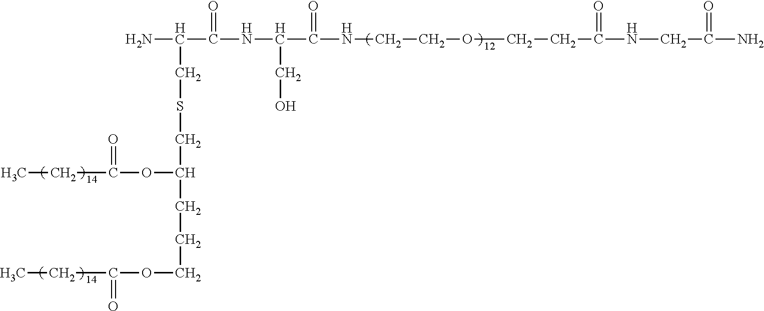 [SMILES: CCC(=O)OCCCC(CSCC(N)C(=O)NC(CO)C(=O)NCCOCCC(=O)NCC(N)=O)OC(=O)CC] | A205 |
| 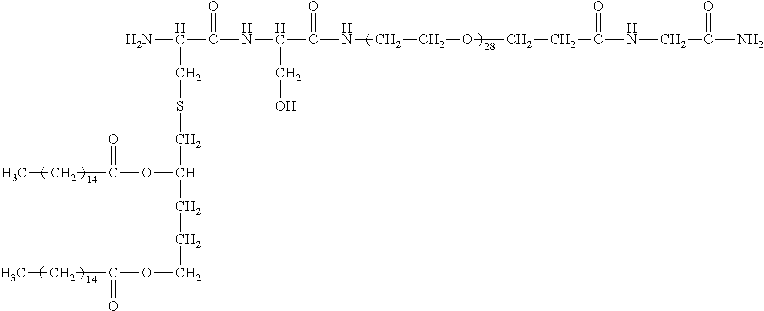 [SMILES: CCC(=O)OCCCC(CSCC(N)C(=O)NC(CO)C(=O)NCCOCCC(=O)NCC(N)=O)OC(=O)CC] | A206 |
| 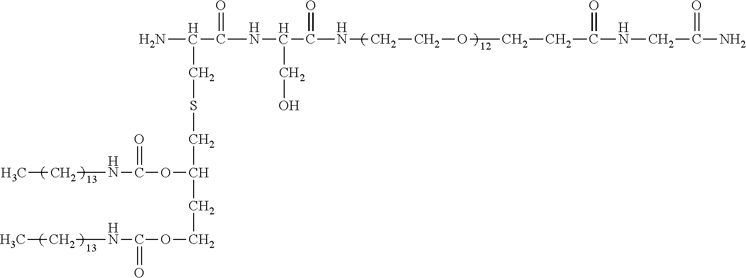 [SMILES: CCNC(=O)OCCC(CSCC(N)C(=O)NC(CO)C(=O)NCCOCCC(=O)NCC(N)=O)OC(=O)NCC] | A207 |
| 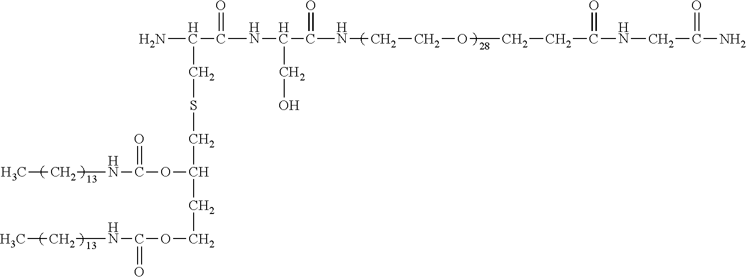 [SMILES: CCNC(=O)OCCC(CSCC(N)C(=O)NC(CO)C(=O)NCCOCCC(=O)NCC(N)=O)OC(=O)NCC] | A208 |
| 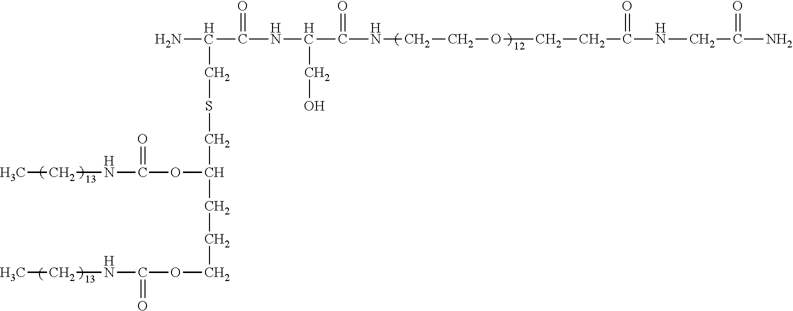 [SMILES: CCNC(=O)OCCCC(CSCC(N)C(=O)NC(CO)C(=O)NCCOCCC(=O)NCC(N)=O)OC(=O)NCC] | A209 |
| 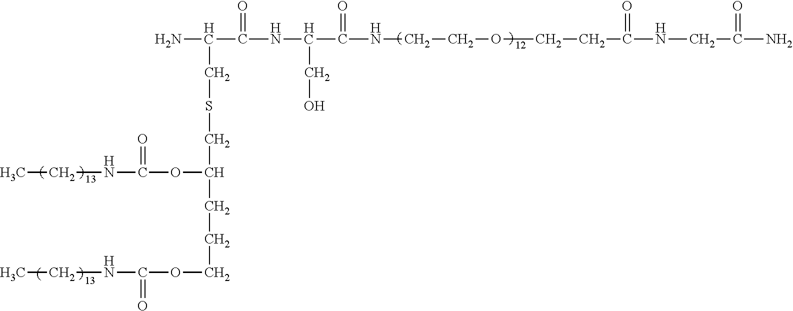 [SMILES: CCNC(=O)OCCCC(CSCC(N)C(=O)NC(CO)C(=O)NCCOCCC(=O)NCC(N)=O)OC(=O)NCC] | A210 |
| 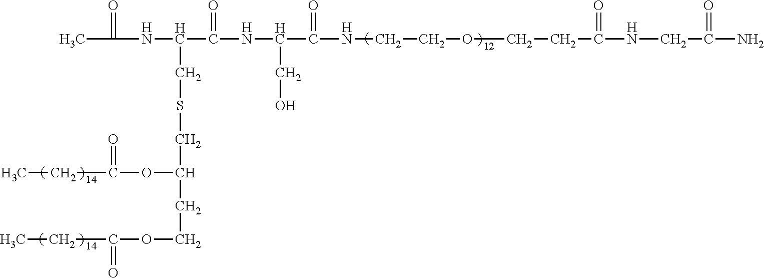 [SMILES: CCC(=O)OCCC(CSCC(NC(C)=O)C(=O)NC(CO)C(=O)NCCOCCC(=O)NCC(N)=O)OC(=O)CC] | A211 |
| 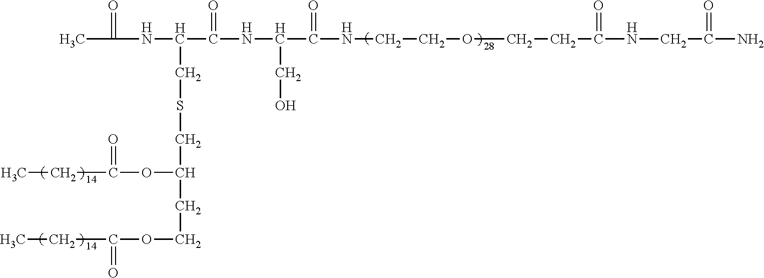 [SMILES: CCC(=O)OCCC(CSCC(NC(C)=O)C(=O)NC(CO)C(=O)NCCOCCC(=O)NCC(N)=O)OC(=O)CC] | A212 |
| 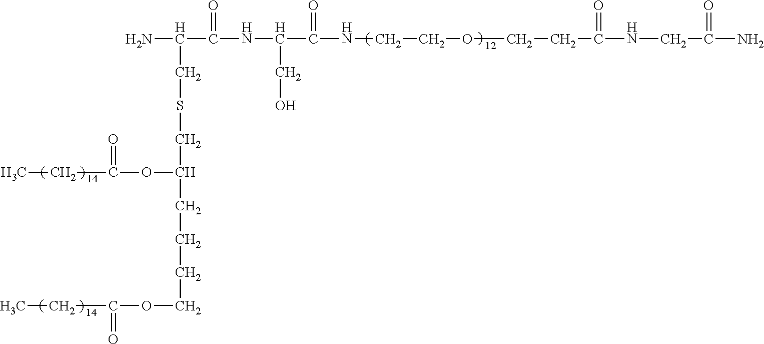 [SMILES: CCC(=O)OCCCCC(CSCC(N)C(=O)NC(CO)C(=O)NCCOCCC(=O)NCC(N)=O)OC(=O)CC] | A213 |
| 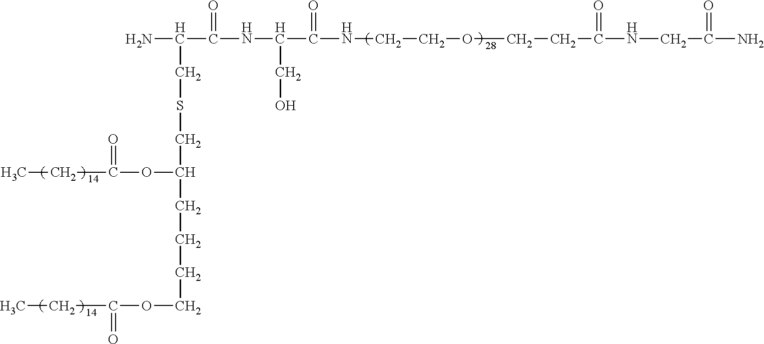 [SMILES: CCC(=O)OCCCCC(CSCC(N)C(=O)NC(CO)C(=O)NCCOCCC(=O)NCC(N)=O)OC(=O)CC] | A214 |
| 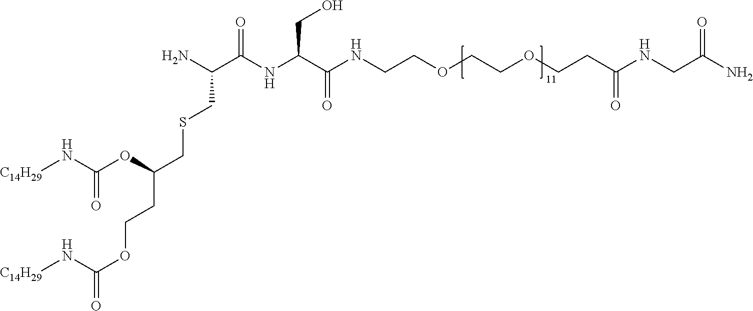 | A215 |
| 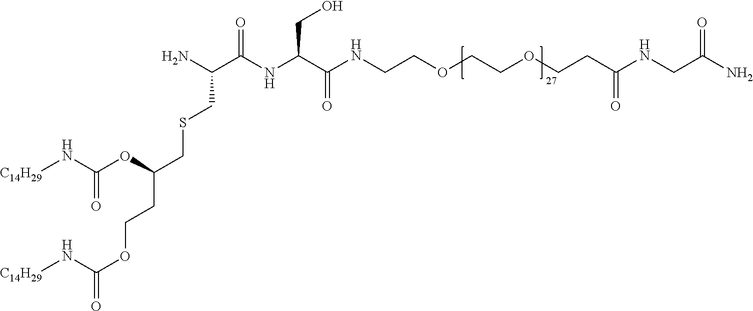 | A216 |
| 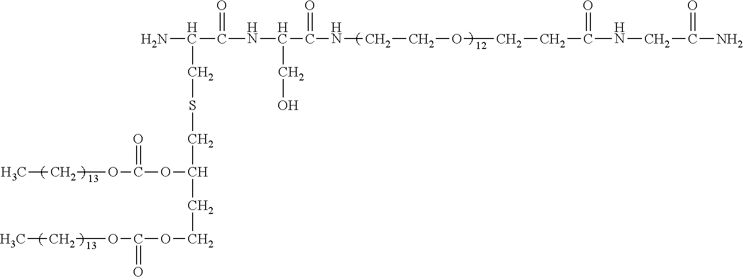 [SMILES: CCOC(=O)OCCC(CSCC(N)C(=O)NC(CO)C(=O)NCCOCCC(=O)NCC(N)=O)OC(=O)OCC] | A217 |
| 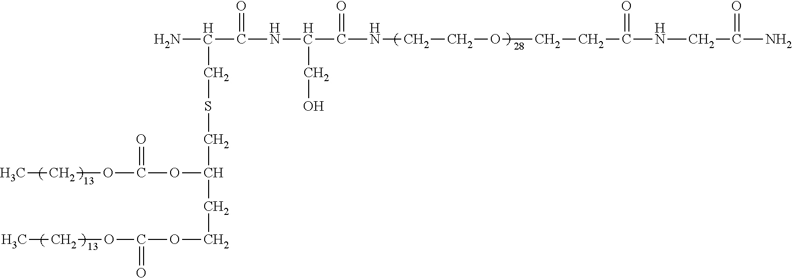 [SMILES: CCOC(=O)OCCC(CSCC(N)C(=O)NC(CO)C(=O)NCCOCCC(=O)NCC(N)=O)OC(=O)OCC] | A218 |
| 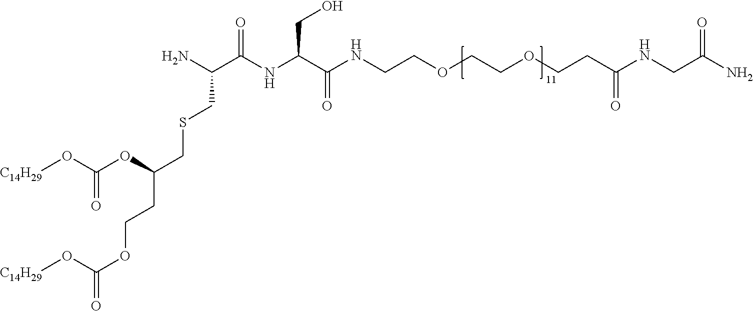 | A219 |
| 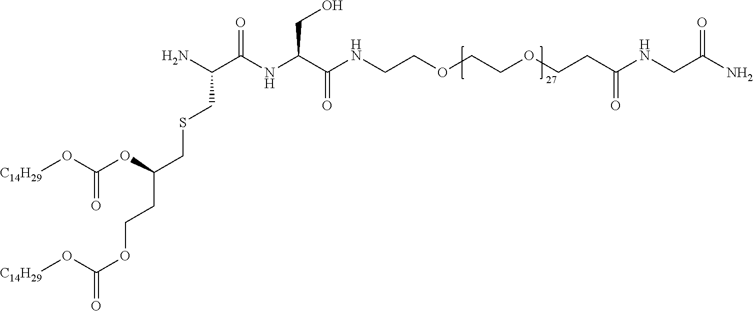 | A220 |
| 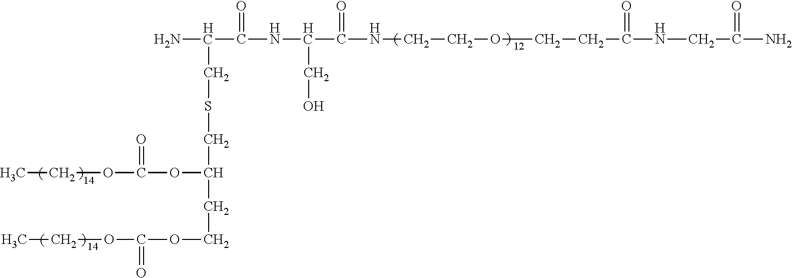 [SMILES: CCOC(=O)OCCC(CSCC(N)C(=O)NC(CO)C(=O)NCCOCCC(=O)NCC(N)=O)OC(=O)OCC] | A221 |
| 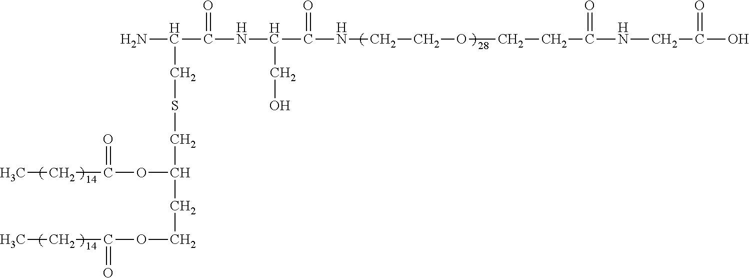 [SMILES: CCC(=O)OCCC(CSCC(N)C(=O)NC(CO)C(=O)NCCOCCC(=O)NCC(=O)O)OC(=O)CC] | A222 |
| 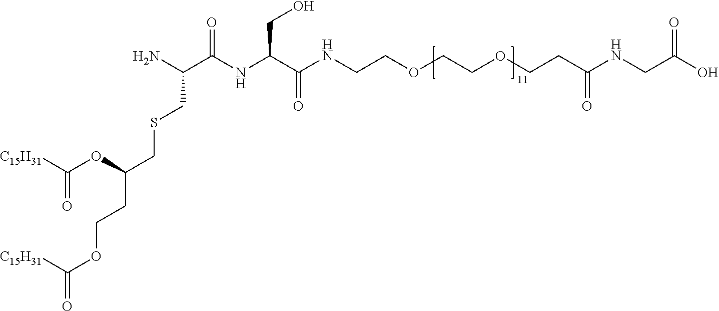 | A223 |
| 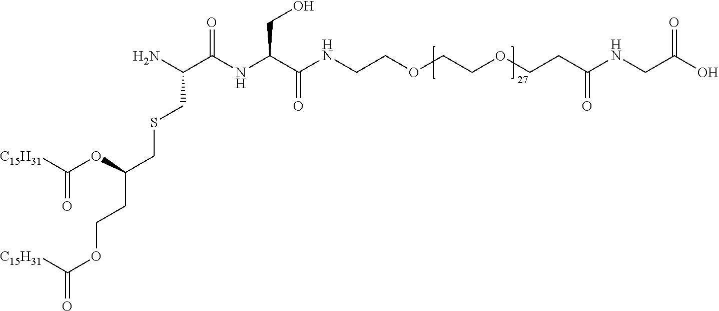 | A224 |
| 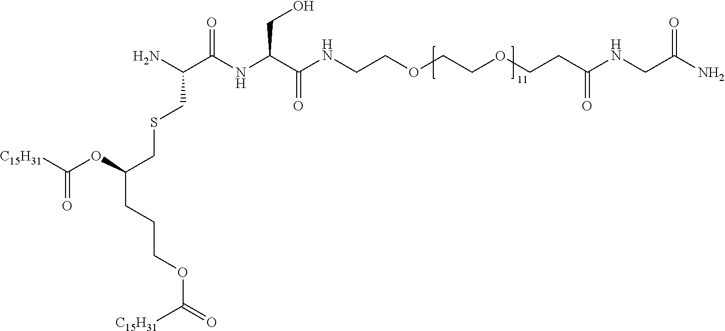 | A225 |
| 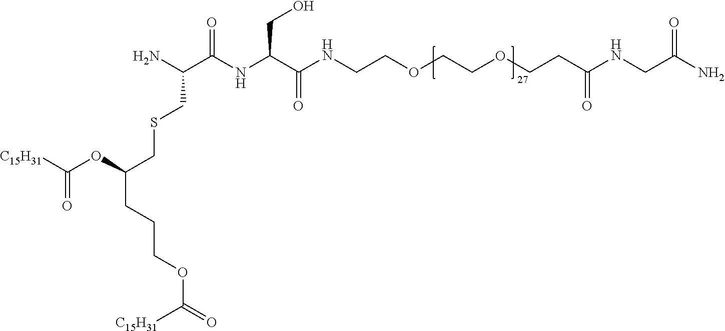 | A226 |
| 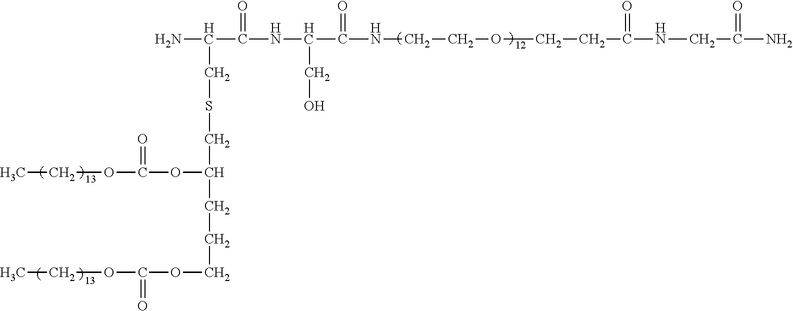 [SMILES: CCOC(=O)OCCCC(CSCC(N)C(=O)NC(CO)C(=O)NCCOCCC(=O)NCC(N)=O)OC(=O)OCC] | A227 |
| 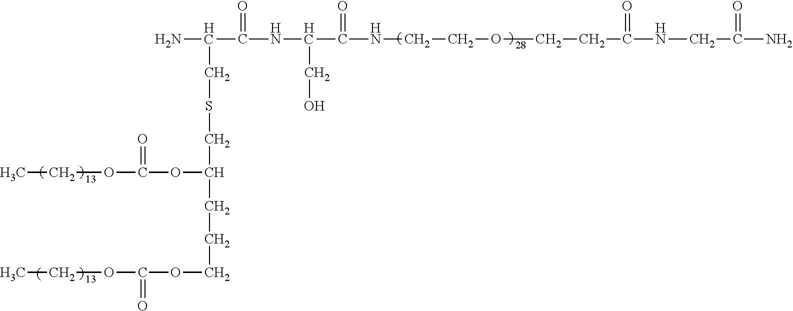 [SMILES: CCOC(=O)OCCCC(CSCC(N)C(=O)NC(CO)C(=O)NCCOCCC(=O)NCC(N)=O)OC(=O)OCC] | A228 |
| 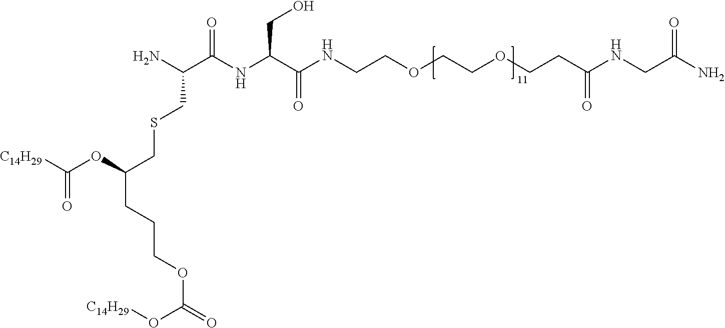 | A229 |
| 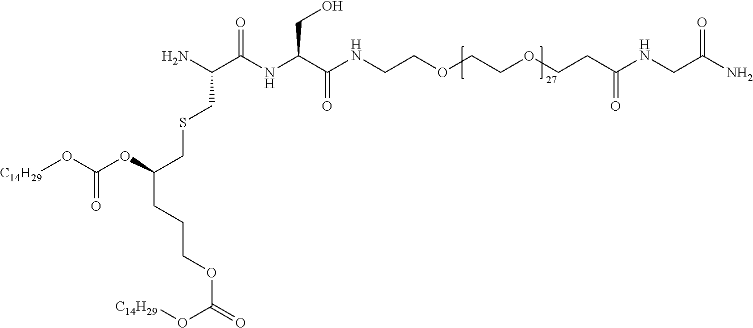 | A230 |
| 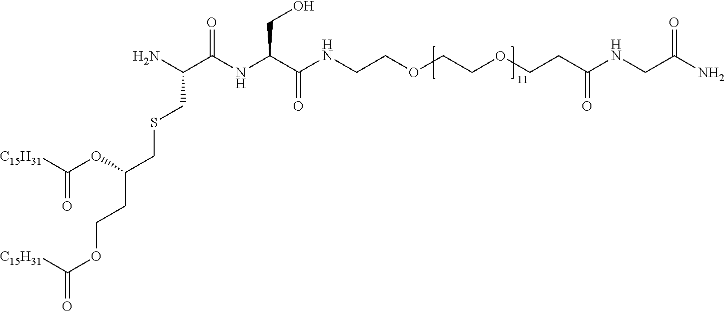 | A231 |
| 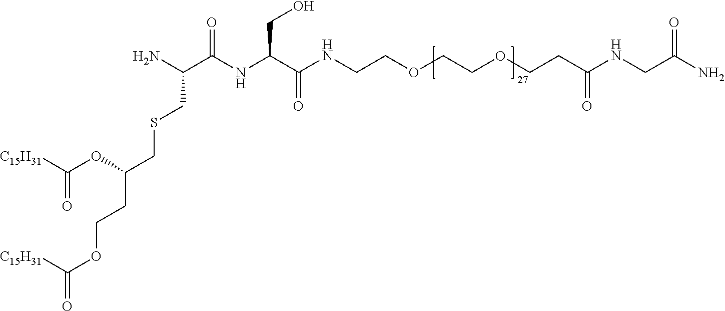 | A232 |

or a pharmaceutically acceptable salt, solvate or prodrug thereof.

35. A method according to any one of claims 1 to 34, further comprising identifying a subject having cancer.

36. The method according to claim 34, wherein the compound is defined by the following formula:

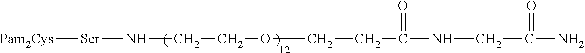

37. The method according to claim 34, wherein the compound is defined by the following formula:

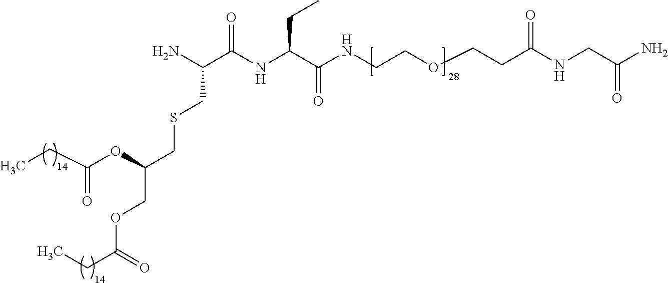

38. A method according to any one of claims 1 to 37, wherein treating cancer is inhibiting metastasis.

39. A method according to claim 38, wherein metastasis is to the lung.

40. Use of a therapeutically effective amount a compound as defined in of any one of claims 1 to 37 in the preparation of a medicament for treating, preventing or minimising progression of cancer in a subject.

41. A method or use according to any one of claims 1 to 40, wherein the cancer is selected from the group consisting of breast cancer, colorectal cancer, adenocarcinomas, mesothelioma, bladder cancer, prostate cancer, germ cell cancer, hepatoma/cholongio carcinoma, neuroendocrine cancer, pituitary neoplasm, small round cell tumour, squamous cell cancer, melanoma, atypical fibroxanthoma, seminomas, nonseminomas, stromal leydig cell tumours, Sertoli cell tumours, skin tumours, kidney tumours, testicular tumours, brain tumours, ovarian tumours, stomach tumours, oral tumours, bladder tumours, bone tumours, cervical tumours, esophageal tumours, laryngeal tumours, liver tumours, lung tumours, vaginal tumours or Wilm's tumour.

42. A method or use according to claim 41, wherein the cancer is melanoma, breast cancer, fibrosarcoma or colon cancer.

43. A method or use according to any one of claims 1 to 42, wherein the compound is administered in a composition that further comprises a pharmaceutically acceptable carrier, diluent or excipient.

44. A method or use according to any one of claims 1 to 43, wherein the compound or composition is suitable for administration, or is administered, intravenously to the subject.

45. The method or use according to any one of claims 1 to 43, wherein the compound or composition is suitable for administration, or is administered, to the respiratory tract of the subject, preferably by inhalation.

46. A compound according to any one of claims 1 to 37 or the composition of claim 43 for use in treating, preventing or minimising progression of cancer in a subject.

47. Use of a compound according to any one of claims 1 to 37 or the composition of claim 43 in treating, preventing or minimising progression of cancer in a subject.

48. The compound of claim 46, the composition of claim 43 or the use of claim 47, wherein the compound is defined by the following formula:

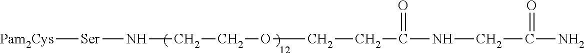

49. The compound of claim 46, the composition of claim 43 or the use of claim 47, wherein the compound is defined by the following formula:

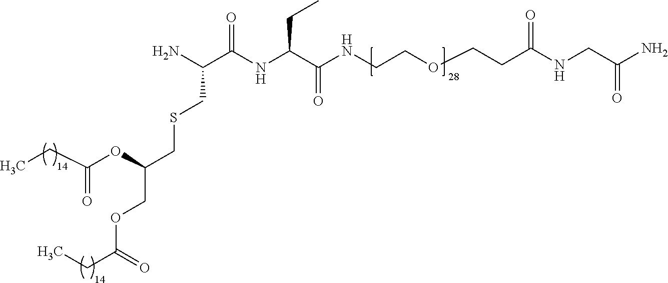

50. The compound of claim 46, 48 to 49 or the use of claim 47, wherein the therapeutically effective amount of a compound is suitable for administration to the respiratory tract of the subject, preferably by inhalation.

51. The compound or use of claim 50, wherein the composition is formulated as a nasal spray or as nasal drops.

52. A kit comprising a compound according to any one of claims 1 to 37 or a composition of claim 43 for use in treating, preventing or minimising progression of cancer in a subject.
